(19) United States
(12) Patent Application Publication
(10) Pub. No.: US 2026/0297609 A1
(43) Pub. Date: Oct. 1, 2026

(54) HSC70-4 IN HOST-INDUCED AND SPRAY-INDUCED GENE SILENCING

(71) Applicant: INSTITUT PASTEUR, PARIS (FR)

(72) Inventors: Maria Carla SALEH, PARIS (FR); Sabrina Johanna FLETCHER, TURIN (IT)

(21) Appl. No.: 18/996,521

(22) PCT Filed: Jul. 28, 2023
(86) PCT No.: PCT/IB2023/000435

§ 371 Date: Jan. 17, 2025

Related U.S. Application Data
(60) Provisional application No. 63/393,166, filed on Jul. 28, 2022.

(51) Int. Cl. C12N 15/82; C07K 14/435

(52) U.S. Cl. CPC C12N 15/8286; C07K 14/43581; C12N 15/8205; C12N 15/8218

(57) ABSTRACT

This disclosure provides proteins, DNA, and dsRNA to improve host-induced gene silencing (HIGS) and spray-induced gene silencing (SIGS) in plant/insect systems, by applying to said plants a composition inducing in same the expression of an Hsc70-4 protein, preferably of a Drosophila melanogaster Hsc70-4 protein.

### FIELD

This disclosure is in the fields of host-induced gene silencing (HIGS), which refers to the silencing of genes in pathogens and pests by expressing homologous double-stranded RNAs (dsRNA) in the host plant, and spray-induced gene silencing (SIGS), which silences genes via an effective RNA spraying method.

### BACKGROUND

Systemic immunity is a defense mechanism that is triggered to fight pathogen infections while contributing to resistance to infection in non-infected cells or tissues¹⁻³. This type of immune response is critical to maintain homeostasis. One of the main characteristics of systemic immunity is the transmission of a signal from infected cells/tissues to non-infected cells/tissues to trigger a broader immune response^{2,3}. During the infection process conserved pathogen-associated motifs known as pathogen-associated molecular patterns (PAMPs) are recognized by a variety of pattern recognition receptors that initiate immune responses⁴. For example, in D. melanogaster, peptidoglycans from Gram-negative bacteria are sensed by peptidoglycan recognition proteins, resulting in activation of the IMD pathway to produce antimicrobial peptides². In vertebrates, intracellular viral dsRNA is sensed by receptors such as endosomal transmembrane toll-like receptors⁵ and the RIG-I-like receptors RIG-I and MDA5^{6,7}. Recognition of dsRNA by these receptors induces a transcriptional program that generates an antiviral state characterized by the secretion of cytokines such as type I interferon that can act in autocrine and/or paracrine fashions to induce the transcription of interferon-stimulated genes. Interferon-like cytokines have not yet been identified in insects. Instead, insects rely on RNAi as the main intracellular antiviral response⁸. Specifically, the siRNA pathway has been found to be the main antiviral defense mechanism in insects⁹. During their replication cycles, viruses produce dsRNA intermediates¹⁰. Sensing and cleavage of virus-derived dsRNA into siRNAs by intracellular proteins triggers the RNAi-mediated immune response to control viral replication. The antiviral siRNA pathway has been most extensively studied in the model insect, Drosophila melanogaster¹¹⁻¹⁴. Specifically, in vivo experiments in adult flies revealed that virus-derived dsRNA is transmitted from infected cells to non-infected cells, where it is processed into siRNAs and confers non-infected cells with pre-exposure protection against Drosophila C virus (DCV) or Sindbis virus infection¹⁵.

Even though intracellular RNAi processes are well understood, the release, transmission, detection, and internalization of dsRNA is still very poorly characterized. While the release of dsRNA from infected cells is thought to be a consequence of cell lysis caused by viral infection¹⁵, how dsRNA is internalized by non-infected cells is poorly understood. Hemocytes isolated from both larvae and adult flies can internalize naked extracellular dsRNA, causing activation of the siRNA pathway^{16,17}. Accordingly, in vitro experiments using the hemocyte-like D. melanogaster Schneider 2 (S2) cell line revealed that these cells can also internalize dsRNA by receptor-mediated endocytosis under certain conditions^{18,19}. These findings imply the existence of a receptor(s) that binds naked extracellular dsRNA at the cell surface prior to internalization. Several proteins have already been proposed as dsRNA receptors in other organisms, such as the systemic RNAi-defective-2 protein (SID-2) in C. elegans²⁰, and the scavenger receptor class A (SR-A) and macrophage-1 antigen (Mac-1) in humans²¹⁻²³. In D. melanogaster, two scavenger receptors, SR-CI and Eater¹⁹, have been proposed as mediators of dsRNA internalization. However their specific roles as dsRNA receptors were not demonstrated.

Host-induced gene silencing (HIGS) technology has emerged as a powerful alternative to chemical treatments for protecting plants from pathogens or pests. HIGS can most effectively be induced by dsRNA-producing transgene constructs. HIGS is an RNAi-based process where small RNAs made in the plant silence the genes of the pests or pathogens that attack the plant. The small RNAs are generally made by producing double stranded RNA (dsRNA) in transgenic plants. Alternatively, dsRNA can be sprayed onto the plant and picked up by either the plant or the pest. Indeed, spray-induced gene silencing (SIGS) is a current, potential, non-transformative, and environment-friendly pest and pathogen management strategy, where naked or nanomaterial-bound dsRNA are sprayed on leaves to cause selective knockdown of pathogenicity genes. The inventors aimed to identify the cell surface protein(s) responsible for binding extracellular dsRNA prior to its internalization in insects.

### SUMMARY OF THE INVENTION

Cellular uptake of dsRNA from the environment is a widespread phenomenon among insects and other pests (e.g., in nematodes). In Drosophila and in honeybees, injection and ingestion, respectively, of dsRNA against a viral sequence is sufficient to hinder replication of the corresponding virus. The inventors aimed to identify cell surface proteins that bind to extracellular dsRNA, mediating its internalization. The inventors thus developed an unconventional co-immunoprecipitation protocol followed by proteomics analysis. The inventors found that silencing Hsc70-4, a member of the Heat-shock protein family, impairs dsRNA internalization. Hsc70-4 localizes to the cell membrane, despite lacking a predicted transmembrane domain. Cell membrane localization was preserved when Hsc70-4 was expressed in mammalian cells, suggesting a conserved role at the cell surface. Furthermore, Hsc70-4 shows an undescribed dsRNA-specific binding capacity. Hsc70-4 is a key element of the dsRNA internalization process and its detailed study may facilitate the development of RNAi-based technologies for pest and vector borne diseases control.

The invention is based on the unexpected result that double stranded RNA (dsRNA) designed to target a mRNA transcribable from a pest insect essential gene may be more toxic to other pest when the plant and/or the pest expresses increased levels of Drosophila melanogaster Hsc70-4 protein, or the plant comprises or is sprayed with Drosophila melanogaster Hsc70-4 protein. Thus, Hsc70-4 protein can be used to control pest infestation of a plant. Thus, in one embodiment, the invention provides a transgenic plant expressing Hsc70-4 protein or both Hsc70-4 protein and a double stranded RNA (dsRNA) molecule comprising a sense strand and an antisense strand, wherein a nucleotide sequence of the antisense strand is complementary to a portion of a mRNA polynucleotide transcribable from a pest gene described herein, wherein the dsRNA molecule is toxic to a pest, such as an insect pest, particularly to a coleopteran and/or a hemipteran pest insect.

In a first aspect, the present invention concerns a recombinant DNA polynucleotide comprising a coding polynucleotide operably linked to a promoter that is functional in a plant cell, wherein said polynucleotide encodes an insect heat shock protein cognate 4 (Hsc70-4), or a functional variant or fragment thereof. This polynucleotide will be referred to as the “polynucleotide of the invention”.

In a second aspect, the present invention concerns an isolated protein, or a functional variant or fragment thereof, expressed by the recombinant DNA polynucleotide of the invention. This isolated protein will be referred to as the “protein of the invention” or “polypeptide of the invention”.

In a third aspect, the present invention concerns a transgenic plant, seed, or plant cell comprising the recombinant DNA polynucleotide of the invention, wherein the polynucleotide encodes a protein whose amino acid sequence shares at least 80% identity to the amino acid sequence of any insect Hsc70-4, preferably to any one of SEQ ID NOs: 1 through 10, wherein the protein may further comprise a His-tag, and wherein the protein may further comprise an heterologous signal peptide for extracellular secretion.

Preferably, said transgenic plant, seed, or plant cell is capable of host-induced gene silencing of a pest, e.g. of an insect.

Preferably, said transgenic plant, seed, or plant cell further comprises a nucleic acid comprising an expressable RNA interference construct encoding a dsRNA molecule capable of down-regulating or suppressing the expression of at least one gene of a pathogen that is capable of infecting the plant; wherein the plant expresses the dsRNA.

In a fourth aspect, the present invention relates to a method of increasing a pest disease resistance (e.g. of an insect disease resistance) in a plant, seed, or plant cell comprising expressing in a plant, plant seed, or plant cell the polypeptide of the invention, wherein the plant, plant seed, or plant cell expressing said polypeptide has increased disease resistance in the plant, plant seed, or plant cell when compared to a control plant, plant seed, or plant cell not expressing said polypeptide.

Preferably, the method further comprises obtaining a progeny plant derived from the plant expressing said polynucleotide, wherein said progeny plant comprises in its genome the polynucleotide and exhibits increased disease resistance when compared to a control plant not comprising the polynucleotide.

Preferably, the genome of the plant, plant seed, or plant cell of the invention has been modified using a genome modification technique selected from the group consisting of a polynucleotide-guided endonuclease, CRISPR-Cas endonucleases, base editing deaminases, a zinc finger nuclease, a transcription activator-like effector nuclease (TALEN), and engineered site-specific meganucleases, or Argonaute.

Preferably, the method of the invention further comprises growing the seed to produce a second-generation progeny plant that comprises the polypeptide and backcrossing the second-generation progeny plant to the second plant to produce a backcross progeny plant that comprises the polypeptide and produces backcrossed seed with increased pest resistance.

In a fifth aspect, the present invention relates to pest inhibitory composition, e.g. to an insect inhibitory composition, comprising the polynucleotide of the invention or the protein of the invention. This composition will be referred to hereafter as the “composition of the invention” or the “pest inhibitory composition” of the invention.

The present invention also relates to a plant part comprising the polynucleotide of the invention or the protein of the invention, wherein the plant part is (a) a seed, a boll, a leaf, a flower, a stem, a root, or any portion thereof; or (b) said plant part is a non-regenerable portion of said seed, boll, leaf, flower, stem, or root.

In a further aspect, the present invention relates to a method of controlling a pest, e.g., an insect pest, comprising contacting said (insect) pest, and/or a plant susceptible to disease caused by the (insect) pest, with an (insect) inhibitory amount of a combination of a polynucleotide of the invention or the protein of the invention and a dsRNA.

Preferably, in this method, the polynucleotide of the invention or the protein of the invention and/or a the dsRNA are applied to the (insect) pest and/or plant by supplementing the diet of the (insects) pests via spraying onto leaves and other commonly eaten parts of the plant of an effective pest-controlling amount; and, optionally, wherein the plant is capable of spray-induced gene silencing; submergence or soaking of root systems in dsRNA and/or Hsc70-4 protein solutions and subsequent uptake by the phloem; or nanoparticle- and Agrobacterium-mediated delivery systems.

The invention also relates to a method of controlling an (insect) pest comprising exposing the pest to the transgenic plant, seed, or plant cell of the invention, thereby controlling the (insect) pest.

The invention also relates to a commodity product derived from the transgenic plant, seed, or plant cell of the invention, wherein said product is selected from the group consisting of whole or processed seeds, beans, grains, kernels, hulls, meals, grits, flours, sugars, starches, protein concentrates, protein isolates, waxes, oils, extracts, juices, concentrates, liquids, syrups, feed, silage, fiber, paper or other food or product produced from plants; wherein optionally said product is non-regenerable.

In a further aspect, the present invention targets a method of making a plant resistant to an (insect) pest infestation, comprising the steps of introducing a recombinant polynucleotide encoding the protein of the invention (Hsc70-4) into a plant cell; regenerating from said plant cell a transgenic plant expressing a pest/insect inhibitory amount of the protein of the invention (Hsc70-4); and demonstrating (insect) pest infestation resistance as a property of said transgenic plant; wherein said plant optionally: (a) is selected from the group consisting of a dicot plant and a monocot plant; (b) is selected from the group consisting of alfalfa, almont, banana, barley, bean, beet, broccoli, cabbage, brassica, brinjal, carrot, cassava, castor, cauliflower, celery, chickpea, Chinese cabbage, celery, citrus, coconut, coffee, corn, clover, cotton, a cucurbit, cucumber, Douglas fir, eggplant, eucalyptus, flax, garlic, grape, guar, hops, leek, legumes, lettuce, Loblolly pine, millets, melons, nectarine, nut, oat, okra, olive, onion, ornamental, palm, pasture grass, papaya, pea, peach, peanut, pepper, pigeonpea, pine, potato, poplar, pumpkin, Radiata pine, radish, rapeseed, rice, rootstocks, rye, safflower, shrub, sorghum, Southern pine, soybean, spinach, squash, strawberry, sugar beet, sugarcane, sunflower, sweet corn, sweet gum, sweet potato, switchgrass, tea, tobacco, tomato, triticale, turf grass, watermelon, and wheat; and/or (c) comprises a supplemental agent selected from the group consisting of an (insect) pest inhibitory protein different from said Hsc70-4, an (insect) pest inhibitory dsRNA molecule, and an (insect) pest inhibitory chemistry; and wherein said inhibitory chemistry is selected from the group consisting of pyrethrins and synthetic pyrethroids; oxadizine derivatives; chloronicotinyls; nitroguanidine derivatives; triazoles; organophosphates; pyrrols; pyrazoles; phenyl pyrazoles; diacylhydrazines; biological/fermentation products; and carbamates.

The invention further relates to a method of reducing resistance development to an interfering RNA molecule in an (insect) pest population, said method comprising increasing the level of Hsc70-4 in the (insect) pest population.

The invention further relates to a method of reducing resistance development to an interfering RNA molecule in an (insect) pest population, the method comprising expressing a protein of the invention in a transgenic plant fed upon by the (insect) pest population, and an interfering RNA which inhibits expression of a target gene in a larval and adult (insect) pest, thereby reducing resistance development in the (insect) pest population compared to an (insect) pest population exposed to an interfering RNA molecule capable of inhibiting expression of a target gene in a larval or adult (insect) pest in the absence of the protein of the invention.

Preferably, in all these aspects and embodiments, the pest is an insect selected from the orders Orders Coleoptera (beetles), Lepidoptera (moths, butterflies), Diptera (flies), Protura, Collembola (springtails), Diplura, Microcoryphia (jumping bristletails), Thysanura (bristletails, silverfish), Ephemeroptera (mayflies), Odonata (dragonflies, damselflies), Orthoptera (grasshoppers, crickets, katydids), Phasmatodea (walkingsticks), Grylloblattodea (rock crawlers), Mantophasmatodea, Dermaptera (earwigs), Plecoptera (stoneflies), Embioptera (web spinners), Zoraptera, Isoptera (termites), Mantodea (mantids), Blattodea (cockroaches), Hemiptera (true bugs, cicadas, leafhoppers, aphids, scales), Thysanoptera (thrips), Psocoptera (book and bark lice), Phthiraptera (lice; including but not limited to suborders Amblycera, Ischnocera and Anoplura), Neuroptera (lacewings, owlflies, mantispids, antlions), Hymenoptera (bees, ants, wasps), Trichoptera (caddisflies), Siphonaptera (fleas), Mecoptera (scorpion flies), Strepsiptera (twisted winged parasites), Stemorrhyncha (e.g. aphids, whiteflies, and scale insects), Auchenorrhyncha (e.g. cicadas, leafhoppers, treehoppers, planthoppers, and spittlebugs), and Coleorrhyncha (e.g. moss bugs and beetle bugs), Orthroptera (e.g. grasshoppers, locusts and crickets, including katydids and wetas), Thysanoptera (e.g. Thrips), Dermaptera (e.g. Earwigs), Isoptera (e.g. Termites), Anoplura (e.g. Sucking lice), Siphonaptera (e.g. Flea), Trichoptera (e.g. caddisflies), and preferably in the Orders dipterans, coleopterans, hemipterans, lepidopterans, hymenopterans and isopterans; more preferably hemipterans, lepidopterans and coleopterans; more preferably, selected from the group consisting of the coleopteran insect pests Sitophilus oryzae (So; rice weevil), Diabrotica virgifera virgifera (Dv; western com rootworm), Diabrotica undecimpunctata howardi (Du; southern com rootworm), Diabrotica barberi (Db; northern corn rootworm), Phyllotreta armoraciae (Pa; horseradish flea beetle), Phyllotreta nemorum (Pn; turnip flea beetle), Phyllotreta cruciferae (Pu; cmcifer flea beetle), Phyllotreta striolata (Ps; striped flea beetle), Phyllotreta atra (Pt; flea beetle), Psylliodes chrysocephala (Pc; cabbage-stem flea beetle), Meligethes aeneus (Ma; pollen beetle), Ceutorhynchus assimilis (Ca; cabbage seedpod weevil), Leptinotarsa decemlineata (Ld; Colorado potato beetle), and/or the hemipteran insect pest is selected from the group consisting of Nezara viridula (Nv; green stink bug), Euschistus heros (Eh; brown stink bug), and Piezodorus guildinii (Pg; red-banded stink bug.

Preferably, in all these aspects and embodiments, the plant is in the superfamily Viridiplantae, preferably a monocotyledonous or dicotyledonous plant.

### BRIEF DESCRIPTION OF THE FIGURES

FIG. 1: S2Xpress cells internalize specifically dsRNA by endocytosis. (a) Luciferase assays testing silencing by dsRNA internalization in S2naive and S2Xpress cells. Cells were co-transfected with plasmids expressing Firefly and Renilla luciferase. 24 h after, 50 ng of dsRNA against Firefly Luciferase (dsFLuc) or dsCtl (dsGFP) was added to the medium (soaking). For control experiments, dsRNAs were co-transfected with plasmids. Luciferase activity was measured 24 h after plasmid expression induction. Firefly luciferase values were normalized to Renilla luciferase values. Data are shown as mean±SD of Firefly/Renilla ratio relative to control condition (Soak. dsCtl) of 3 independent experiments pooled together. Welch's ANOVA was used to detect significant differences compared to control condition (Soak. dsCtl) (n=8-9). S2naive W(DFn, DFd): 164.8 (4.000, 15.48); S2Xpress W(DFn, DFd):84.16 (4.000, 16.11). (b) Dose-dependent curves of dsRNA internalization were done by luciferase silencing similar as in (a). Different amounts of dsRNA were added during soaking (0.78 ng to 100 ng). Data are shown as mean±SD of Firefly/Renilla ratio relative to control condition (Soak. dsCtl) of 2 independent experiments pooled together. Welch's ANOVA was used to detect significant differences compared to control condition (Soak. dsCtl) for each cell type (n=6). Specific p-values can be found in FIG. 6e. S2naive W(DFn, DFd): 72.09 (12.00, 23.61); S2Xpress W(DFn, DFd): 36.63 (12.00, 24.32). (c) Luciferase assays to test effect of culture medium in dsRNA internalization. Experiments were done as in (a) with modifications. Culture medium was change after 4 h to normal growing medium. Data are shown as mean±SD of Firefly/Renilla ratio relative to control condition (Soak. dsCtl) of 3 independent experiments pooled together. Welch's ANOVA was used to detect significant differences compared to control condition (Soak. dsCtl) (n=9). S2naive Insect-Xpress medium W(DFn, DFd): 202.1 (4.000, 16.00), S2naive 10% FBS Schneider's insect medium W(DFn, DFd): 232.2 (4.000, 16.00), S2Xpress Insect-Xpress medium W(DFn, DFd): 644.3 (4.000, 16.00), S2Xpress 10% FBS Schneider's insect medium W(DFn, DFd): 959.7 (4.000, 16.00). (d) Internalization of dsRNA by S2naive and S2Xpress cells was evaluated by fluorescence confocal imaging. Cells were soaked with 30 ng of dsRNA (dsFLuc) labeled with Cy3 (magenta) for 40 min. Actin is in green and nuclei are in blue. For Dynasore experiments, inhibitor was added for 1 h before soaking with dsRNA. (e) To test the specificity of internalization by S2Xpress cells, dsRNA (dsFLuc), dsDNA (FLuc) or siRNA (GAPDH) labeled with Cy3 (magenta) was added during soaking. Experiments were performed as in (d). ALU: arbitrary light units; Soak.: soaking; Tfx.: transfection. Confocal images were taken at 630× magnification. Scale bars represent 5 μM. p-values<0.05 were considered significant * p<0.05; ** p<0.01; **** p<0.0001.

FIG. 2: Proteomics of cell surface proteins and dsRNA binding proteins. (a) Protocol scheme of purification of cell surface proteins of S2naive and S2Xpress cells. Pierce™ Cell Surface Biotinylation and Isolation Kit was used to biotinylate and purify cell surface proteins. Protein identification was done by LC-MS/MS. (b) Venn diagram showing common found proteins between S2naive and S2Xpress. (c) Cellular component analysis of found proteins for both cells types, showing the percentage of proteins found corresponding to the principal cell parts. Statistical analysis was done by FunRich software⁵⁷, p-values were calculated by Hypergeometric test. (d) Protocol scheme of immunoprecipitation (IP) of dsRNA-binding proteins in S2Xpress cells. Dynasore inhibitor was used to prevent internalization but not binding of dsRNA (labeled with Cy3) to the cell membrane. dsRNA-Cy3 was crosslinked to proteins by UV (254 nm), and an IP was performed using the anti-Cy3 antibody. Precipitated proteins were identified by LC-MS/MS. (e) Venn diagram showing common proteins between the 2 different proteomic protocols. (f) Cellular component and molecular function analysis of proteins found in the IP against dsRNA-Cy3 show percentage of proteins corresponding to principal cell parts and relevant molecular functions respectively in S2 Xpress. Venn diagrams, cellular component and molecular function analysis were done with FunRich software⁵⁷. Protocol schemes were created with BioRender.com.

FIG. 3: High content screen of selected candidates. (a) 24 selected candidates were silenced in S2Xpress cells by transfecting with specific dsRNA for 72 h. dsCtl and Dynasore wells were transfected with unspecific dsRNA (dsFLuc). For Dynasore wells, inhibitor was added for 10 min before soaking with 30 ng of unspecific dsRNA (dsFLuc) labeled with Cy3 for 30′. Plates were imaged on Opera Phenix High content microscope at 630× magnification. A script was designed on Columbus software to quantify intensity and number of spots inside the cells. Histogram show mean±SD of number of spots/cell. One-way ANOVA was used to detect significant differences compared to control condition (dsCtl) (Ctl- and dsCtl n=8; dsCandidates and Dyansore n=3, dsCG3373 n=2) (F (DFn, DFd): 19.75 (26, 63)). All p-values can be found in FIG. 8b. (b) High content analysis similar as (a) just for Hsc70-4 (CG4264). Histograms show mean±SD of number of spots/cell and median intensity compared to control condition (dsCtl) of 3 independent experiments pooled together (Ctl-n=24; Dynasore n=9; dsCtl n=23, dsCG4264 n=23). For median intensity, Ctl-values were subtracted from other conditions, and values were relativized to dsCtl. Welch's ANOVA was used to detect significant differences compared to control condition (dsCtl) for number of spots/cell (W(DFn, DFd): 38.93 (3.000, 23.18)), and median intensity (W(DFn, DFd): 66.30 (2.000, 35.14)). (c) Confocal imaging was done to confirm High content imaging findings. S2xpress were transfected with dsHsc70-4 or dsCtl for 72 h, and 30 ng of dsRNA (dsFLuc) labeled with Cy3 (magenta) was added to the medium for 40 min before imaging. Actin is in green, and nuclei are in blue. Experiment was repeated 3 times. Confocal images were taken at 630× magnification. Scale bars represent 5 M. (d) RT-qPCR comparing the expression levels of Hsc70-4 between S2naive and S2Xpress cells. Rp49 was used as a housekeeping gene. Data are shown as mean±SD of fold change relative to S2naive of 3 independent experiments pooled together. Unpaired t-test was used to detect significant differences (n=9; t=9.972, df=16). p-values<0.05 were considered significant. * p<0.05; *** p<0.001; **** p<0.0001.

FIG. 4: Cell surface localization of rHsc70-4. (a) Hsc70-4 coding region was cloned into pAc5-V5/His Drosophila expressing plasmid. S2naive and S2Xpress cells were transfected with 500 or 1000 ng of plasmid or without plasmid, and incubated for 48 h (upper panel) or 1, 2, and 3 days (lower panel). For lower panel, cells were co-transfected with 500 ng of plasmid and 100 ng of dsHsc70-4 or dsCtl (dsFLuc) to check silencing at the protein level. RIPA was used to extract proteins. Equal volumes of samples were loaded on an SDS-PAGE (upper panel). For lower panel, 5 μg of proteins were loaded per lane. After electrophoresis, immunoblotting was performed with anti-V5. α-Tubulin was used as a loading control. The band corresponds to the predicted molecular weight (74.7 kDa) for Hsc70-4. (b) S2naive cells and S2Xpress were transfected with 100 ng of plasmid expressing Hsc70-4 for 48 h to see cellular localization. Cells were then fixed and blocked/permeabilized with 10% normal goat serum (NGS)-0.2% Triton X-100 or 10% NGS for non-permeabilized wells. rHsc70-4 was detected by immunofluorescence with anti-V5 (green). Actin is in magenta and nuclei are in blue. (c) S2naive cells and S2Xpress were transfected with 100 ng of plasmid expressing Hsc70-4 for 48 h. After, 30 ng of dsRNA (dsFLuc) labeled with Cy3 (magenta) was added for 40 min. Then, cells were fixed and blocked/permeabilized with 10% NGS-0.2% Triton X-100 or 10% NGS for non-permeabilized wells. rHsc70-4 was detected by immunofluorescence with anti-V5 (green). Nuclei are in blue. Confocal images were taken at 630× magnification. Scale bars represent 5 μM. dpt: days post transfection.

FIG. 5: rHsc70-4 binds to dsRNA but not dsDNA or siRNA. (a) To test binding, different concentrations of recombinant Hsc70-4 (0.25 μM, 0.5 μM, 1 μM, 2 μM, 3 μM, 4 μM and 5 μM) were incubated with dsRNA probe (dsFLuc labeled with Cy3, 0.76 nM). Samples were incubated for 30 min at 25° C., and loaded on a 4% native polyacrylamide gel. Electrophoresis was done at 200 V on ice for 45 min. Gel was developed by fluorescence detection on a Typhoon™ FLA 9000. Binding was visualized by a shift in the mobility of the detected probe compared to probe alone (first lane). For competition assay, 10 times more of unlabeled dsRNA (dsFLuc) was added to the mix (Concentration of rHsc70-4 used was 1 μM). An increased on the mobility of the detected probe confirmed that unlabeled dsRNA displaced the labeled-probe. (b) DNA (FLuc) labeled with Cy3 was used as probe. DNA-Cy3 (0.76 nM) was incubated with different concentrations of rHsc70-4 (0.5 μM, 1 μM, 2 μM), and incubated as in (a). Gel was run for 80 min and developed as in (a). (c) siRNA (GAPDH) labeled with Cy3 was used as probe. siRNA-Cy3 (0.76 nM) was incubated with different concentrations of rHsc70-4 (0.5 μM, 1 μM, 2 μM), and incubated as in (a). Gel was run for 18-20 min and developed as in (a). All EMSA experiments were performed twice, giving similar results. First lane in all gels corresponds to free probe.

FIG. 6: dsRNA internalization by different S2 cell lines. (a) Luciferase assays to test internalization of dsRNA by S2naive, S2R+, S2p FHV and S2p FHV+DAV cells. Cells were co-transfected with plasmids expressing Firefly and Renilla luciferase. 48 h after, 25, 50 or 100 ng of dsRNA targeting Firefly Luciferase (dsFLuc) or dsCtl (dsGFP) was added to the medium (soaking) to allow internalization and silencing of Firefly luciferase. For control experiments, dsRNAs were co-transfected with plasmids. Luciferase activity was measured 48 h after plasmid expression induction. Firefly luciferase values were normalized to Renilla luciferase values. Data are shown as mean±SD of Firefly/Renilla ratio relative to control condition (Soak. dsCtl) of 2 experiments for S2naive, S2p FHV and S2p FHV+DAV (n=5-6), and 1 experiment for S2R+ (n=3). Welch's ANOVA was used to detect significant differences compared to control condition (Soak. dsCtl) for S2naive, S2p FHV and S2p FHV+DAV experiments, and one-way ANOVA was used for S2R+ experiment. p-values<0.05 were considered significant. S2naive W(DFn, DFd): 279.7 (5.000, 13.05); S2R+F (DFn, DFd): 244.9 (5, 12); S2p FHV W(DFn, DFd): 1276 (5.000, 12.54); S2p FHV+DAV W(DFn, DFd): 184.7 (5.000, 11.83). (b) Agarose gel of labeled and unlabeled dsRNA (dsGL3) with Cy3. (c) Immunofluorescence of S2Xpress cells with the anti-dsRNA antibody J2 (green) to confirm that labeling of dsRNA (dsGL3) with Cy3 (magenta) was efficient, and that the signal detected was in fact dsRNA, and not free Cy3. Cells were soaked with 30 ng of dsRNA-Cy3 for 40 min. DAPI was used to stain nuclei (blue). Confocal images showed complete co-localization between Cy3 signal and J2, confirming that the Cy3 signal comes from the dsRNA-Cy3. (d) dsGFP labeled with Cy3 (magenta) was used to confirm that uptake of dsRNA by S2Xpress cells was not sequence-dependent. Soaking with dsRNA was done similar as in (c). Actin is in green and nuclei are in blue. For Dynasore experiments, inhibitor was added for 20 min before soaking with dsRNA. Confocal images were taken at 630× magnification. Scale bars represent 5 μM. *** p<0.001; **** p<0.0001.

FIG. 7: Cell surface proteins comparison and dsRNA binding protein IP controls. (a) Aliquots of proteins purified by the biotinylation of cell surface proteins of S2naive and S2Xpress were ran on a SDS-PAGE, followed by silver staining. INPUT samples refer to lysed samples prior to isolation. ELUTION samples refer to isolated proteins that were further analyzed by LC-MS/MS. Differences between S2naive and S2Xpress cells were evidenced by the different banding pattern on the ELUTION lanes. Negative control samples were done by not adding biotin during labeling. (b) Immunofluorescence with anti-Cy3 was done to check specificity of antibody to be used in the IP of dsRNA binding proteins of S2Xpress cells. 30 ng of dsRNA (dsFLuc) labeled with Cy3 (magenta) was added to the cells for 40 min. Dynasore inhibition was done by adding the inhibitor for 1 h prior to soaking. Immunofluorescence was done with anti-Cy3 (green). Nuclei are in blue. Confocal images were taken at 630× magnification. Complete co-localization of dsRNA-Cy3 and anti-Cy3, even when Dynasore was added, confirmed that this antibody is highly specific, and thus a good option for the IP. (c) Uptake of dsRNA-Cy3 (magenta) was done as in (b) with minor modifications. Actin was stained with phalloidin (green) and DAPI was used for nuclei (blue). Arrow points to a dying/dead cells with high Cy3 signal. dsRNA seems to bind to these death nuclei/cells, resulting in the immunoprecipitation of nuclear proteins. Confocal images were taken at 630× magnification. Scale bars represent 5 μM.

FIG. 8: High content screen intensity and silencing confirmation. (a) 24 selected candidates were silenced in S2Xpress cells by transfecting with 10 ng of specific dsRNA for 72 h. dsCtl and Dynasore wells were transfected with unspecific dsRNA (dsFLuc). For Dynasore wells, inhibitor was added for 10 min before soaking. After, 30 ng of unspecific dsRNA (dsFLuc) labeled with Cy3 were added to the medium for 30′. Plates were imaged on Opera Phenix High content microscope at 630× magnification. Script was designed on Columbus software to quantify intensity and number of spots inside the cells. Histogram show mean±SD of number median intensity relative to control condition (dsCtl). Ctl-values were subtracted from other conditions, and values were relativized to dsCtl. One-way ANOVA was used to detect significant differences compared to control condition (dsCtl) (dsCtl n=8; dsCandidates and Dyansore n=3, dsCG3373 n=2) (F (DFn, DFd): 7.536 (25, 56)). (b) To confirm silencing of Hsc70-4 by dsHsc70-4, S2Xpress were transfected with 50 ng dsHsc70-4 or dsCtl (dsFLuc) for 72 h (triplicates per condition). After, RNA was extracted, quantified, and cDNA with Oligo(dT)₁₈ primers was produced with equal amounts of RNA. PCR was done with primers flanking the dsRNA targeting region. PCR products were run on a 1% agarose gel with ethidium bromide. Rp49 was used as a loading and PCR control. Silencing was confirmed by the decreased intensity of the bands from cells treated with dsHsc70-4. Experiment was done twice. p-values<0.05 were considered significant. * p<0.05; ** p<0.01; ****p<0.0001.

FIG. 9: SR-CI and Eater receptors are not involved in uptake of dsRNA in S2Xpress cells. (a) Agarose gel of PCR product used to further produce by in vitro transcription dsRNA targeting SR-CI and Eater. Two sets of primers were used for each receptor. Flies cDNA was used a positive control for the primers. Rp49 was used as loading and PCR control. The inventors were not able to amplify SR-CI when cDNA from S2Xpress was used. Eater PCR amplicon was purified and dsRNA was done by in vitro transcription (dsEater). (b) High content imaging was used to test the effect of silencing Eater on the uptake of dsRNA. S2Xpress cells were transfected with 10 ng of dsEater to silence the receptor for 72 h. dsCtl and Dynasore wells were transfected with unspecific dsRNA (dsFLuc). For Dynasore wells, inhibitor was added for 10 min before soaking. After, 30 ng of unspecific dsRNA (dsFLuc) labeled with Cy3 were added for 30′. Plates were imaged on Opera Phenix High content microscope at 630× magnification. Script was designed on Columbus software to quantify intensity and number of spots inside the cells. Histograms show mean±SD of number of spots/cell and median intensity compared to control condition (dsCtl) of 2 independent experiments pooled together (Ctl-n=15; Dynasore n=7; dsCtl n=15, dsEater n=7). For median intensity, Ctl-values were subtracted from other conditions, and values were relativized to dsCtl. Welch's ANOVA was used to detect significant differences compared to control condition (dsCtl) for number of spots/cell (W(DFn, DFd): 47.77 (3.000, 16.14)), and for median intensity (W(DFn, DFd): 66.61 (2.000, 11.32)). p-value<0.05 was considered significant. (c) Confocal imaging was done to test the effect of silencing Eater on uptake of dsRNA by S2Xpress cells. S2xpress were transfected with dsEater or not transfected (Control) for 24 h. After, 30 ng of dsRNA (dsFLuc) labeled with Cy3 (magenta) was added to the medium for 40 min. Actin is in green and nuclei are in blue. Confocal images were taken at 630× magnification. Scale bars represent 5 μM.

FIG. 10: rHsc70-4 localizes to the cell surface of Hela cells. Hsc70-4 coding region was cloned into pcDNA6-myc/His mammalian expression vector. Hela cells were transfected with 1000 ng of plasmid or without plasmid, and incubated for 48 h. RIPA was used to extract proteins. 4 μg of proteins were loaded on an SDS-PAGE. After electrophoresis, immunoblotting was performed with anti-6× His. α-Tubulin was used as a loading control. The band corresponds to the predicted molecular weight (75.4 kDa) for rHsc70-4. (b) HeLa cells were transfected with 100 ng of plasmid expressing Hsc70-4 for 48 h. After, cells were fixed and blocked/permeabilized with 10% NGS-0.2% Triton X-100 or 10% NGS for non-permeabilized wells. rHsc70-4 was detected by immunofluorescence with anti-6× His (green) ON at 4° C. Actin is in magenta and nuclei are in blue. Confocal images were taken at 400× magnification. Scale bars represent 15 μM.

FIG. 11: Binding of rHsc70-4 to dsRNA is not sequence dependent. To test if binding was sequence dependent, a different labeled-dsRNA was used (dsRNA-2 corresponds to dsCG6647). rHsc70-4 (2 μM) was incubated with dsRNA-2 probe labeled with Cy3, (0.76 nM). Samples were incubated for 30 min at 25° C., and loaded on a 4% native polyacrylamide gel. Electrophoresis was done at 200 V on ice for 45 min. Gel was developed by fluorescence detection on a Typhoon™ FLA 9000. Binding was visualized by a shift in the mobility of the detected probe compared to probe alone (first lane). For competition assay, 10 times more of unlabeled dsRNA-2 (dsCG6647) was added to the mix. An increased on the mobility of the detected probe confirmed that unlabeled dsRNA displaced the labeled-probe. Experiment was done twice giving similar results. First lane corresponds to free probe.

### DETAILED DESCRIPTION

In order for the present disclosure to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided. The term “consisting of” excludes any element, step, or ingredient not specified in the claim. In re Gray, 53 F.2d 520, 11 USPQ 255 (CCPA 1931); Ex parte Davis, 80 USPQ 448, 450 (Bd. App. 1948) (“consisting of” defined as “closing the claim to the inclusion of materials other than those recited except for impurities ordinarily associated therewith”). The term “consisting essentially of” limits the scope of a claim to the specified materials or steps “and those that do not materially affect the basic and novel characteristic(s)” of the claimed disclosure.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” may mean within one or more than one standard deviation per the practice in the art. “About” or “approximately” may mean a range of up to 10% (i.e., ±10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The disclosure may, as the skilled person will recognize, be performed by using alternative features. Similarly, features introduced by “in an embodiment of the disclosure” or similar expressions are intended to be optional features, without any restriction regarding further embodiments of the disclosure, without any restrictions regarding the scope of the disclosure and without any restriction regarding the possibility of combining the features introduced in such way with other optional or non-optional features of the disclosure.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International d'Unités (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “TheOxford Dictionary Of Biochemistry And Molecular Biology”, Cammack et al. eds., 2nd ed, (2006), Oxford University Press, provide those of skill in the art with a general dictionary for many of the terms used in this disclosure.

### A. Identification of Hsc70-4 as a Player in dsRNA Internalization by Insect Cells

Cereal grains, including major cereal grains (e.g., wheat and rice) and other minor grains (e.g., barley and oats) have provided over 56% of the caloric and 50% of the protein requirements in human diets for thousands of years, since their domestication. Crop plants are subject to diseases caused by parasitic insects. Although the development of modern agricultural science and technology greatly reduced the yield loss, an average of 10-15% of the global crop production (more than 300 million tons) is still threatened by plant diseases. With the increasing world population, the demand for crop products, combined with food security and balanced nutrition, are rapidly increasing. High-yielding and disease resistant varieties are required at unprecedented levels.

Host-induced gene silencing (HIGS) refers to the silencing of genes in pathogens and pests by expressing homologous double-stranded RNAs (dsRNA) or artificial microRNAs (amiRNAs) in the host plant. Spray-induced gene silencing (SIGS) is a current, potential, non-transformative, and environment-friendly pest and pathogen management strategy, where naked or nanomaterial-bound dsRNA are sprayed on leaves to cause selective knockdown of pathogenicity genes. Although HIGS represents a promising approach for limiting crop losses caused by pathogens and pests, many questions remain, including how silencing RNAs are secreted from plant cells, and how cells in the pathogen or pest take up these RNAs.

To elucidate the process of dsRNA internalization by insect cells, the inventors sought to identify key components involved in the internalization of naked extracellular dsRNA in D. melanogaster. For this purpose, the inventors utilized an in vitro cell model based on the adaptation of S2 D. melanogaster cells to grow in a serum-free medium that gave the inventors' the necessary resolution and tractability to examine this very specific step of whole organism systemic immunity.

The first step of dsRNA internalization is binding of dsRNA to a cell surface protein. Given their different dsRNA internalization capacities, the inventors compared the cell surface protein composition of S2naive and S2Xpress cells. The inventors had found that S2Xpress cells, but not S2naive cells, internalized dsRNA-Cy3. As expected, a significant percentage of the proteins identified by the inventors' proteomic approaches were categorized as membrane proteins. Although the total number of proteins identified was less than expected, the inventors hypothesize that the proteins the inventors did identify may represent the more abundant and/or accessible proteins within the plasma membrane. Interestingly, even though some proteins were present at the surface of both cell types, the majority were unique to the surface of each cell type. This confirms that there are intrinsic differences in the compositions of the plasma membranes of S2naive and S2Xpress cells. Surprisingly, a significant percentage of the proteins the inventors identified in the S2Xpress cells were mitochondrion proteins. This finding may reflect the changes undergone by these cells during adaptation to growth in protein-free media. Several reports show that mitochondrion can translocate to the plasma membrane in yeast and mammalian cells, and in some cases they have been shown to interact with the extracellular milieu^{30,31}. Moreover, one study showed that the plasma membrane can interact with mitochondrion as a response to cellular stress induced by reactive oxygen species by translocating caveolins to facilitate adaptation and homeostasis³². Given that S2Xpress cells are grown without FBS, it is conceivable that they may experience stress due to nutrient deficiency and that changes in the composition of the plasma membrane may have allowed them to adapt to these conditions.

While the inventors' initial proteomic approach based on subcellular localization gave the inventors' valuable information about the protein composition of the surfaces of S2naive and S2Xpress cells, the inventors wanted to add an additional layer of information to the inventors' analyses by identifying proteins present in dsRNA-protein complexes at the cell surface. For this purpose, the inventors utilized the CLIP technique, which is widely used to study RNA-protein interactions³³⁻³⁵ The inventors' state-of-the-art CLIP-based immunoprecipitation protocol was designed to specifically isolate dsRNA-protein complexes present at the cell surface during dsRNA internalization. Indeed, a high percentage of these proteins were already identified as RNA binding proteins, including the DEAD-box RNA helicase Belle, which is involved in RNAi¹⁹. Furthermore, Argonaute-2 (Ago-2), one of the main components of the RNAi pathwayl^{14,36}, was also identified. Although the inventors' protocol was designed to sequester dsRNA at the cell surface by using Dynasore to inhibit internalization, inhibition of internalization was not 100% effective and some dsRNA was expected to become internalized. Thus, it is not surprising that the inventors identified some intracellular proteins known to interact with dsRNA. In fact, the inventors' identification of intracellular dsRNA-binding proteins such as Ago-2 validates the effectiveness of the inventors' immunoprecipitation protocol. Together, the inventors' complementary proteomic approaches identified 15 dsRNA-binding proteins that are present at the surface of S2Xpress cells.

For the inventors' functional screening, the inventors decided to consider all proteins as possible candidates, regardless of whether they contain a known dsRNA binding domain. The inventors' screen of the selected candidates successfully identified one protein, heat-shock cognate 70-4 (Hsc70-4), as necessary for dsRNA internalization. Hsc70-4 is a member of the large Heat-shock protein family³⁷. The heat-shock cognate proteins (Hscs) differ from the more well-known heat-shock proteins (Hsps) in that they are constitutively expressed^{38,39}. The Hsp70 family is comprised of a number of highly homologous proteins primarily known for their roles as chaperones, although they are involved in a wide variety of other cellular processes, including clathrin-mediated endocytosis, protein quality control, RNAi, viral attachment and entry, antiviral defense, and neurotransmitter exocytosis⁴⁰⁻⁴⁴ They contain a highly conserved amino-terminal ATPase domain, a substrate-binding domain, and a less conserved carboxy-terminal domain⁴⁵. Because members of the Hsc70 family display high sequence similarity, it is sometimes assumed that their roles are somewhat interchangeable. The inventors' own experiments show that this is not always the case, since another of the candidate proteins tested, Hsc70-5 (CG8542; 52% sequence identity) was not found to play a role in dsRNA internalization. Accordingly, in one embodiment, the disclosure provides the unexpected identification of Hsc70-4 as an insect protein necessary for dsRNA internalization in pests. In another embodiment, the disclosure provides manipulating Hsc70-4 for improvement of dsRNA uptake into pests and its use in HIGS and SIGS.

Hsc70-4 is a highly conserved protein within the Animalia phylum. It is known to have a crucial role in the nervous system as a synaptic chaperone^{43,46}. It is also involved in autophagy and protein aggregation^{46,47}. Interestingly, experimental data show that Hsc70-4 is involved in clathrin-mediated endocytosis⁴⁵. Considering this, one may hypothesize that the role of Hsc70-4 during dsRNA internalization is mainly through the intracellular endocytic step. However, Hsc70-4 was previously found to participate specifically in the uncoating of clathrin-coated vesicles (CCVs) during endocytosis⁴⁵. The inventors' microscopy-based internalization assay was designed to quantify dsRNA within the interior of the plasma membrane, thus, the inventors would have considered dsRNA as internalized even if it was inside CCVs (but not released into the cytosol due to Hsc70-4 silencing). This suggests that Hsc70-4 may have an additional role at the cell surface during internalization of dsRNA. This is supported by the inventors' observations that rHsc70-4 accumulates at the cell surface, presents an extracellular face, and directly interacts with dsRNA. Thus, the inventors' results suggest that Hsc70-4 could act as a cell surface dsRNA receptor or co-receptor.

The inventors' results indicate that Hsc70-4 is necessary for dsRNA internalization but perhaps not sufficient. S2naive cells, which do not internalize dsRNA under their standard growth conditions, express this protein and rHsc70-4 accumulates at the cell membrane in these cells. Nevertheless, the inventors did not detect Hsc70-4 in the inventors' proteomics analysis of cell surface proteins in S2naive cells. It is possible that S2naive cells express Hsc70-4 at the cell surface, but at levels too low for its detection by the inventors' proteomics approach. It is also possible that another protein inhibits the detection of cell surface Hsc70-4 in S2naive cells. Importantly, the inventors' protein localization assays were done with a recombinant protein and may not necessarily reflect the expression or localization patterns of endogenous Hsc70-4. Finally, different isoforms (7 isoforms have already been identified; source: FlyBase) or post-translation modifications could give different functions to Hsc70-4 in S2Xpress cells compared to S2naive cells. Accordingly, the disclosure provides that extracellular Hsc70-4 can be manipulated to improve dsRNA internalization by (insect) pest cells. In one embodiment, the disclosure provides adding Hsc70-4 protein to plants to promote dsRNA internalization by pests and thus promote insecticidal or pesticidal activity of dsRNAs. In one embodiment, the Hsc70-4 is sprayed onto the surface of the plants. In one embodiment, Hsc70-4 improves SIGS. In one embodiment, different Hsc70-4 isoforms can be used to promote HIGS and SIGS.

How can Hsc70-4 act to internalize dsRNA? A recent report shows that the mammalian orthologue of this protein, HSPA8, acts as a co-factor receptor at the cell surface⁴⁸. Specifically, HSPA8 was found to directly interact with Porcine reproductive and respiratory syndrome virus at the cell surface and was required for virus attachment and entry⁴⁸. Interestingly, the authors showed that an additional protein, CD163, was also required for viral entry. Furthermore, HSPA8 was found to have a dual role during viral entry, as the protein directly binds to viral glycoproteins and also participates in clathrin-mediated endocytosis. Thus, these findings, together with the inventors' experimental data allow the inventors to hypothesize a similar role for Hsc70-4 during dsRNA binding and internalization in D. melanogaster. Moreover, a co-factor role for Hsc70-4 in dsRNA internalization could explain why S2 cells have been shown to internalize only long dsRNA, as opposed to siRNA (21 bp)¹⁸. One can hypothesize that if two cell surface factors, one being Hsc70-4, are necessary for dsRNA binding and internalization, shorter dsRNAs are not long enough to bind to both proteins.

Several insects are able to internalize extracellular dsRNA from the extracellular environment. In this context, mosquitos are of particular relevance since they serve as vectors of several human pathogenic viruses (arboviruses)⁴⁹. Interestingly, injection of dsRNA into the mosquito body cavity is sufficient to silence cognate gene expression^{50,51}.

This indicates that there is a mechanism by which mosquito cells can internalize naked dsRNA. Other insects, such as honey bees, also have the capacity to internalize dsRNA⁵². One particular study in honey bees showed that oral acquisition of dsRNA targeting the Israeli Acute Paralysis Virus is sufficient to confer resistance to the treated bees⁵³. The capacity of insects to internalize dsRNA has facilitated a new RNAi-based pest-control technology in agriculture, commonly termed “host-induced gene silencing”^{52,54,55}. Since this is a promising approach that could eventually prevent substantial agricultural and economic losses, it is vital to acquire a thorough knowledge of the mechanism of action of the internalization of dsRNA, including its target tissues, cell internalization and processing, and transmission. The present work is a starting point that will facilitate the study of dsRNA internalization mechanisms through the identification of one of the key elements of this process. To the inventors' knowledge, this is the first report that shows that Hsc70-4 directly binds dsRNA and that it is present at the cell membrane with an extracellular face. Further studies are needed to characterize the dsRNA binding domain of Hsc70-4, its binding partners, and its structure. Moving forward, the study of endogenous Hsc70-4 as well as its role in vivo will provide more detailed information on the specific mechanisms by which Hsc70-4 promotes dsRNA internalization

### B. Hsc70-4 in Host-Mediated Gene Silencing

The invention is based on the unexpected result that double stranded RNA (dsRNA) designed to target a mRNA transcribable from a (insect) pest essential gene may be more toxic to the (insect) pest when the plant and/or the (insect) pest express increased levels of Drosophila melanogaster Hsc70-4 protein, or the plant comprises or is sprayed with Drosophila melanogaster Hsc70-4 protein. Thus, Hsc70-4 protein can be used to control (insect) pest infestation of a plant. Thus, in one embodiment, the invention provides a transgenic plant expressing Hsc70-4 protein or both Hsc70-4 protein and a double stranded RNA (dsRNA) molecule comprising a sense strand and an antisense strand, wherein a nucleotide sequence of the antisense strand is complementary to a portion of a mRNA polynucleotide transcribable from a (insect) pest gene described herein, wherein the dsRNA molecule is toxic to a (insect) pest, particularly to a coleopteran and/or a hemipteran insect, preferably a hemipteran insect pest selected from the group consisting of Nezara viridula (Nv), Euschistus heros (Eh) and Piezodorus guildinii (Pg). The needs outlined previously are met by the present invention which, in various embodiments, provides new compositions and methods of controlling economically important (insect) pests.

The invention encompasses a nucleic acid construct encoding an Hsc70-4 protein of the disclosure. In one embodiment, the Hsc70-4 protein is encoded by the nucleic acids of SEQ ID NO:23, or SEQ ID NO:100-107, which encode the protein of any one of SEQ ID NO:1 through 10, with or without a protein purification tag (e.g., His Tag), and with or without a signal peptide for extracellular secretion. The invention further encompasses a nucleic acid molecule encoding at least one interfering molecule (e.g., a dsRNA capable of silencing a pest gene) and a nucleic acid encoding an Hsc70-4 protein. This nucleic acid construct is preferably an expression vector. The invention further encompasses a recombinant vector comprising a regulatory sequence operably linked to a nucleotide sequence that encodes a Hsc70-4 protein of the invention and to an interfering RNA molecule. A regulatory sequence may refer to a promoter, enhancer, transcription factor binding site, insulator, silencer, or any other DNA element involved in the expression of a gene. The invention further encompasses chimeric nucleic acid molecules comprising an interfering RNA molecule with an antisense strand of a dsRNA operably linked to a nucleic acid encoding an Hsc70-4 protein.

### C. Hsc70-4

The disclosure provides polypeptides that exhibit at least 80%, 82%, 83%, 84%, 85%, 90%, 91%, 92%, 93%, 93.5%, 94%, 94.5%, 95%, 95.5%, 96%, 96.5%, 97%, 97.5%, 98%, 98.5%, 99%, 99.5%, or 100% identity with the amino acid sequence of an Hsc70-4 protein, preferably of an insect Hsc70-4 protein, preferably of any of the Hsc70-4 polypeptide of SEQ ID Nos 1 through 10. In one embodiment, an Hsc70-4 polypeptide of the present invention contain one or more amino acid sequence modifications compared to one or more of the Hsc70-4 of SEQ ID NOs: 1 through 10, including substitutions and deletions, of amino acid residues are related by amino acid modifications such that the modified polypeptide exhibits enhanced (insect) pest inhibitory activity. In one embodiment the modified polypeptide exhibits enhanced (insect) pest inhibitory activity of at least one dsRNA whose antisense strand is complementary to a portion of a mRNA polynucleotide transcribable from a (insect) gene. one pest In embodiment, the modified polypeptides exhibit enhanced Hemipteran inhibitory spectrum and/or improved Hemipteran inhibitory activity. In one embodiment, the polypeptide is designed to have enhanced resistance to proteases that exist in the hemolymph or (insect) pest gut.

The invention also encompasses functional variants or fragments of the polypeptides as defined herein, e.g. of the Drosophila melanogaster Hsc70-4 polypeptides of SEQ ID NO:1-10.

Accordingly, the encompassed variants and fragments have the same function as the reference Hsc70-4 polypeptide, in particular, they favour the pest inhibitory activity of at least one dsRNA whose antisense strand is complementary to a portion of a mRNA polynucleotide transcribable from a pest gene. Preferably, they enhance the (insect) pest inhibitory activity of at least one dsRNA whose antisense strand is complementary to a portion of a mRNA polynucleotide transcribable from an (insect) pest gene.

Preferred variants are e.g., the orthologs of the Hsc70-4 protein that have been identified in other insects, e.g., in Anopheles gambiae (African malaria mosquito), Aedes aegypti, Anopheles darlingi (American malaria mosquito), Apis florea (dwarf honeybee), Apis mellifera (bee, honey bee, European or Western honey bee), Acyrthosiphon pisum, Bombus impatiens, Bombus terrestris (large earth bumblebee), Bombyx mori (silk moth; silkworm), Cimex lectularius (Hymenoptera), Culex quinquefasciatus (Diptera), Danaus plexippus (Lepidoptera), Dendroctonus ponderosae («Mountain pine beetel»), Drosophila albomicans, Drosophila ananassae, Drosophila arizonae, Drosophila biarmipe, Drosophila bipectinata, Drosophila busckii, Drosophila elegans, Drosophila erecta, Drosophila eugracilis, Drosophila ficusphila, Drosophila grimshawi, Drosophila guanche, Drosophila hydei, Drosophila innubila, Drosophila kikkawai, Drosophila mauritiana, Drosophila miranda, Drosophila mojavensis, Drosophila navojoa, Drosophila novamexicana, Drosophila obscura, Drosophila persimilis, Drosophila yacuba, Drosophila pseudoobscura, Drosophila rhopaloa, Drosophila santomea, Drosophila sechellia, Drosophila serrata, Drosophila simulans, Drosophila subobscura, Drosophila subpulchrella, Drosophila suzukii, Drosophila takahashii, Drosophila teissieri, Drosophila virilis, Drosophila willistoni, Drosophila Yakuba, Glossina morsitans (tsetse fly), Heliconius melpomene (Lepidoptera), Linepithema humile (ant), Lucilia cuprina (Diptera—greenbottle fly), Mayetiola destructor (Diptera), Megachile rotundata (bee), Musca domestica (fly), Nasonia vitripennis (wasp), Rhodnius prolixus (Heteroptera), Zeugodacus cucurbitae (melon fly), Tribolium castaneum (Coleoptera).

Other useful Hsc70-4 variants are the orthologs of the Hsc70-4 protein that have been identified in other organisms (non-insect), e.g., in Danio rerio (Zebrafish), in Caenorhabditis elegans (Nematode, roundworm), in Mus musculus, in Rattus norvegicus, in Xenopus tropicalis, in Arabidopsis thaliana (thale-cress), in Saccharomyces cerevisiae (Brewer's yeast), in Schizosaccharomyces pombe (Fission yeast) or in Escherichia coli (enterobacterium).

It is also possible to use in the composition/methods of the invention any other Hsc70-4 variant that has the same function as the Drosophila melanogaster Hsc70-4 polypeptides, whose sequence shares preferably at least 80% identity with any of SEQ ID NO:1-10.

Due to the highly conserved structure of the Hsc70-4 over animal and insect species (above 80% identity), it is possible to use a functional Hsc70-4 protein of a define pest in order to favour the inhibitory activity of a dsRNA in other pests. For example, it is possible to use the Hsc70-4 protein of Drosophila melanogaster in order to favour the inhibitory activity of a dsRNA active against other insect pests, such as flies, coleopters, etc. and also against non-insect pathogens, such as nematodes, oocmycetes, fungi, etc. Also, it is possible to use the Hsc70-4 protein of non-insect pests (e.g. from Zebrafish, nematodes, oomycetes, etc) in order to favour the inhibitory activity of a dsRNA against insect pests, such as flies, coleopters, Lepidoptera, etc. or against other non-insect pathogens.

The terms “peptide,” “polypeptide,” and “protein” are herein used interchangeably, and refer to a compound comprised of amino acid residues covalently linked by peptide bonds. A protein or peptide contains at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

An “amino acid/s” or an “amino acid residue/s” may be a natural or non-natural amino acid residue/s linked by peptide bonds or bonds different from peptide bonds. The amino acid residues may be in D-configuration or L-configuration (referred to herein as D- or L-enantiomers). An amino acid residue comprises an amino terminal part (NH₃) and a carboxy terminal part (COOH) separated by a central part (R group) comprising a carbon atom, or a chain of carbon atoms, at least one of which comprises at least one side chain or functional group. NH₃ refers to the amino group present at the amino terminal end of an amino acid or peptide, and COOH refers to the carboxy group present at the carboxy terminal end of an amino acid or peptide. The generic term amino acid comprises both natural and non-natural amino acids. Natural amino acids of standard nomenclature are listed in 37 C.F.R. 1.822(b)(2). Examples of non-natural amino acids are also listed in 37 C.F.R. 1.822(b)(4), other non-natural amino acid residues include, but are not limited to, modified amino acid residues, L-amino acid residues, and stereoisomers of D-amino acid residues. Naturally occurring amino acids may be further modified, e.g. amidation, hydroxyproline, γ-carboxyglutamate, and O-phosphoserine.

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89 (22): 10915.

One of ordinary skill in the art is familiar with a variety of methods to calculate identity or homology of one polypeptide to the other. In one embodiment, to calculate percent identity, the sequences being compared are typically aligned in a way that gives the largest match between the sequences. One example of a computer program that may be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span,” as determined by the algorithm.) In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm. In some embodiments, identity may be determined as percentage of identity using known computer algorithms such as the “FASTA” program, using for example, the default parameters as in Pearson et al. (1988) Proc. Natl. Acad. Sci. USA 85:2444 (other programs include the GCG program package (Devereux, I, et al., Nucleic Acids Research 12 (1): 387 (1984)), BLASTP, BLASTN, FASTA Atschul, S. F., et al, J Molec Biol 215:403 (1990); Guide to Huge Computers, Mrtin J. Bishop, ed., Academic Press, San Diego, 1994, and Carillo et al. (1988) SIAM J Applied Math 48:1073). For example, the BLAST function of the National Center for Biotechnology Information database may be used to determine identity. Other commercially or publicly available programs include, DNAStar “MegAlign” program (Madison, Wis.) and the University of Wisconsin Genetics Computer Group (UWG) “Gap” program (Madison Wis.)).

The term “similarity” and “similar” and grammatical variations thereof, as used herein, mean that an amino acid sequence contains a limited number of conservative amino acid substitutions compared to a peptide reference sequence, e.g. the variant peptide versus the parent peptide as defined herein. A variety of criteria can be used to indicate whether amino acids at a particular position in a peptide are similar. In making changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

Substitutions may be conservative or non-conservative amino acid substitutions. A “conservative substitution” is the replacement of one amino acid by a biologically, chemically or structurally similar residue. Biological similarity means that the substitution does not destroy a biological activity. Structural similarity means that the amino acids have side chains with similar length, such as alanine, glycine and serine, or a similar size. Chemical similarity means that the residues have the same charge, or are both either hydrophilic or hydrophobic.

For example, a conservative amino acid substitution is one in which an amino acid residue is replaced with an amino acid residue having a similar side chain, for example amino acids with basic side chains (e.g., lysine, arginine, histidine); acidic side chains (e.g., aspartic acid, glutamic acid) uncharged polar side chains e.g., glycine, asparagine, glutamine, serine, threonine, tyrosine, cysteine, histidine) nonpolar side chains (e.g., alanine, valine, leucine, isoleucine, proline, phenylalanine, methionine, tryptophan); beta-branched side chains (e.g., threonine, valine, isoleucine), and aromatic side chains (e.g., tyrosine, phenylalanine, tryptophan). Particular examples include the substitution of one hydrophobic residue, such as isoleucine, valine, leucine or methionine, for another, or the substitution of one polar residue for another, such as the substitution of arginine for lysine, glutamic acid for aspartic acid, or glutamine for asparagine, serine for threonine, and the like. Proline, which is considered more difficult to classify, shares properties with amino acids that have aliphatic side chains (e.g., Leu, Val, He, and Ala). In certain circumstances, substitution of glutamine for glutamic acid or asparagine for aspartic acid may be considered a similar substitution in that glutamine and asparagine are amide derivatives of glutamic acid and aspartic acid, respectively.

The disclosure also provides for variants of the specific parent polypeptides identified above namely those of SEQ ID NO:1-10 A variant, which is also termed a “variant polypeptide” or “modified polypeptide” herein, is a polypeptide that is derived from but not identical to a parent Hsc70-4 polypeptide as defined herein. A variant polypeptide may include a number of variations compared to the parent polypeptide as defined herein, for example to increase or decrease physical or chemical properties of the parent polypeptide as defined herein, for example to decrease its ability to resist oxidation, to improve or increase solubility in aqueous solution, to decrease aggregation, to decrease synthesis problems, etc. “Variants” is intended to mean substantially similar sequences. For polynucleotides, a variant comprises a polynucleotide having a deletion (i.e., truncations) at the 5′ and/or 3′ end and/or a deletion and/or addition of one or more nucleotides at one or more internal sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the Hsc70-4 polypeptides provided herein. Naturally occurring variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis or gene synthesis but which still encode an Hsc70-4 polypeptide. Biologically active variants of an Hsc70-4 polypeptide disclosed herein (and the polynucleotide encoding the same) will have at least about 80%, 81%, 82%, 83%, 84%, 85%, 90%, 91%, 92%, 93%, 93.5%, 94%, 94.5%, 95%, 95.5%, 96%, 96.5%, 97%, 97.5%, 98%, 98.5%, 99%, 99.5%, or more sequence identity to the polypeptide of any one of SEQ ID NO:1-10 as determined by sequence alignment programs and parameters described elsewhere herein

In one embodiment, the variant may for instance include one or more deletions of amino acid residues from the N- and/or C-terminal end of the parent peptide as defined herein one or more additions of amino acid residues to the N- and/or C-terminal of the parent peptide as defined herein and/or one or more amino acid substitutions, additions or deletions within the amino acid sequence of the parent peptide as defined herein. One type of variant is a “derivative”, where chemical modifications are introduced, for instance in the side-chains of one of more of the amino acid residues of the parent peptide's amino acid sequence (thus effectively resulting in a peptide that includes an amino acid residue substitution relative to the parent peptide as defined herein). A derivative can also include a chemical modification that involves the N-terminal amino group and/or the C-terminal COOH group. Derivatives are described in more detail herein. It is important to note that some derivatives of the parent peptides as defined herein, are those that could be obtained by substituting an amino acid residue with another naturally occurring amino acid residue, whereas other derivatives involve chemical modifications that result in the provision of peptides that could not be encoded by a nucleic acid sequence.

In one embodiment, the disclosure provides compositions comprising isolated Hsc70-4 polypeptide, variants, or fragments thereof. The terms “isolated or purified” mean modified “by the hand of humans” from the natural state; in other words if an object exists in nature, it is said to be isolated or purified if it is modified or extracted from its natural environment or both. For example, a polynucleotide or a protein/peptide naturally present in a living organism is neither isolated nor purified; on the other hand, the same polynucleotide or protein/peptide separated from coexisting molecules in its natural environment, obtained by cloning, amplification and/or chemical synthesis is isolated for the purposes of the present disclosure. Furthermore, a polynucleotide or a protein/peptide which is introduced into an organism by transformation, genetic manipulation or by any other method, is “isolated” even if it is present in said organism. The term purified as used in the present disclosure means that the proteins/peptides according to the disclosure is essentially free from, contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms. By definition, isolated peptides are also non-naturally occurring, synthetic peptides. Methods for isolating or synthesizing peptides of interest with known amino acid sequences are well known in the art are essentially free of association with the other proteins or polypeptides, as is for example the product purified from the culture of recombinant host cells or the product purified from a nonrecombinant source.

The invention also provides recombinant nucleic acids encoding Hsc70-4 of SEQ ID NO:1-10 or variants thereof. In one embodiment, the nucleic acid has the sequence of SEQ ID NO:23, or SEQ ID NO:100-107. As used herein, a “recombinant” polynucleotide comprises a combination of two or more chemically linked nucleic acid segments which are not found directly joined in nature. By “directly joined” is intended the two nucleic acid segments are immediately adjacent and joined to one another by a chemical linkage. In specific embodiments, the recombinant polynucleotide comprises a polynucleotide of interest or active variant or fragment thereof such that an additional chemically linked nucleic acid segment is located either 5′, 3′ or internal to the polynucleotide of interest. Alternatively, the chemically linked nucleic acid segment of the recombinant polynucleotide can be formed by the deletion of a sequence. The additional chemically linked nucleic acid segment or the sequence deleted to join the linked nucleic acid segments can be of any length, including for example, 1, 2, 3, 4, 5, 6, 7, 8, 9, 10, 15, 20 or greater nucleotides. Various methods for making such recombinant polynucleotides are disclosed herein, including, for example, by chemical synthesis or by the manipulation of isolated segments of polynucleotides by genetic engineering techniques. In specific embodiments, the recombinant polynucleotide can comprise a recombinant DNA sequence or a recombinant RNA sequence.

In one embodiment, the disclosure provides recombinant Hsc70-4 polypeptides, variants, or fragments thereof. A “recombinant polypeptide” comprises a combination of two or more chemically linked amino acid segments which are not found directly joined in nature. In specific embodiments, the recombinant polypeptide comprises an additional chemically linked amino acid segment that is located either at the N-terminal, C-terminal or internal to the recombinant polypeptide. Alternatively, the chemically-linked amino acid segment of the recombinant polypeptide can be formed by deletion of at least one amino acid. The additional chemically linked amino acid segment or the deleted chemically linked amino acid segment can be of any length, including for example, 1, 2, 3, 4, 5, 6, 7, 8, 9, 10, 15, 20 amino acids.

Methods and compositions are provided which employ polynucleotides and polypeptides having Hsc70-4 activity. Moreover, any given variant or fragment of an Hsc70-4 sequence may further comprise an improved catalytic capacity in the presence of the inhibitor glyphosate when compared to an appropriate control. Fragments and variants of the Hsc70-4 polynucleotides and polypeptides provided herein are also encompassed by the present disclosure. By “fragment” is intended a portion of the polynucleotide or a portion of the amino acid sequence and hence protein encoded thereby. Fragments of a polynucleotide may encode protein fragments that retain Hsc70-4 activity, and in specific embodiments, can further comprise an improved property such as improved HIGS. Alternatively, fragments of a polynucleotide that are useful as hybridization probes or PCR primers generally do not encode fragment proteins retaining biological activity. In specific embodiments, a fragment of a recombinant polynucleotide or a recombinant polynucleotide construct comprises at least one junction of the two or more chemically linked or operably linked nucleic acid segments which are not found directly joined in nature. Thus, fragments of a nucleotide sequence may range from at least about 20 nucleotides, about 50 nucleotides, about 100 nucleotides, about 200 nucleotides, about 300 nucleotides, about 400 nucleotides, about 500 nucleotides, about 600 nucleotides, about 700 nucleotides, about 800 nucleotides, about 900 nucleotides, about 1000 nucleotides, about 1-100 nucleotides, about 1-200 nucleotides, about 1-300 nucleotides, and up to the full-length polynucleotide encoding the Hsc70-4 polypeptides. A fragment of an Hsc70-4 polynucleotide that encodes a biologically active portion of an Hsc70-4 protein of the disclosure will encode at least 25, 50, 75, 100, 125, 150, 175, 200, 225, 250, 275, 300, 325, 350, 375, 400, or 425 amino acids, or up to the total number of amino acids present in a full-length Drosophila melanogaster (Fruit fly) Hsc70-4 polypeptide, which is 651, minus one.

The use of the term “polynucleotide” is not intended to limit a polynucleotide of the disclosure to a polynucleotide comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the disclosure also encompass all forms of sequences including, but not limited to, single-stranded forms, double-stranded forms, hairpins, stem-and-loop structures, and the like. For example, a polynucleotide construct may be a recombinant DNA construct. A “recombinant DNA construct” comprises two or more operably linked DNA segments which are not found operably linked in nature. Non-limiting examples of recombinant DNA constructs include a polynucleotide of interest or active variant or fragment thereof operably linked to heterologous sequences which aid in the expression, autologous replication, and/or genomic insertion of the sequence of interest. Such heterologous and operably linked sequences include, for example, promoters, termination sequences, enhancers, etc., or any component of an expression cassette; a plasmid, cosmid, virus, autonomously replicating sequence, phage, or linear or circular single-stranded or double-stranded DNA or RNA nucleotide sequence; and/or sequences that encode heterologous polypeptides. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide.

In one embodiment, a fragment of an HSC70-4 polynucleotide may encode a biologically active portion of an Hsc70-4 polypeptide, or it may be a fragment that can be used as a hybridization probe or PCR primer using methods disclosed below. A biologically active portion of an Hsc70-4 polypeptide can be prepared by isolating a portion of one of the Hsc70-4 polynucleotides, expressing the encoded portion of the Hsc70-4 polypeptides (e.g., by recombinant expression in vitro), and assessing the activity of the Hsc70-4 portion of the Hsc70-4 protein. Polynucleotides that are fragments of a Hsc70-4 nucleotide sequence comprise at least 20, 50, 100, 200, 300, 400, 500, 600, 700, 800, 900, 1000, 1 100, 1200, 1300 contiguous, 1900 nucleotides, or up to the number of nucleotides present in a full-length Hsc70-4 polynucleotide disclosed herein, which is 1962, minus one. In specific embodiments, such polypeptide fragments are active fragments, and in still other embodiments, the polypeptide fragment comprises a recombinant polypeptide fragment. As used herein, a fragment of a recombinant polypeptide comprises at least one of a combination of two or more chemically linked amino acid segments which are not found directly joined in nature.

Further provided and better described below are engineered host cells that are transduced (transformed or transfected) with one or more Hsc70-4 sequences or active variants or fragments thereof. The Hsc70-4 polypeptides or variants and fragments thereof can be expressed in any organism, including in non-animal cells such as plants, yeast, fungi, bacteria and the like. Details regarding non-animal cell culture can be found in Payne et al. (1992) Plant Cell and Tissue Culture in Liquid Systems, John Wiley & Sons, Inc. New York, NY; Gamborg and Phillips (eds.) (1995) Plant Cell, Tissue and Organ Culture; Fundamental Methods Springer Lab Manual, Springer-Verlag (Berlin, Heidelberg, New York); and Atlas and Parks (eds.) The Handbook of Microbiological Media (1993) CRC Press, Boca Raton, FL. Plants, plant cells, plant parts and seeds, and grain having the Hsc70-4 sequences disclosed herein are also provided. In specific embodiments, the plants and/or plant parts have stably incorporated at least one heterologous Hsc70-4 polypeptide disclosed herein or an active variant or fragment thereof. In addition, the plants or organism of interest can comprise multiple Hsc70-4 polynucleotides (i.e., at least 1, 2, 3, 4, 5, 6 or more). Additional host cells of interest can be a eukaryotic cell, an animal cell, a protoplast, a tissue culture cell, prokaryotic cell, a bacterial cell, such as E. coli, B. subtilis, Streptomyces, Salmonella typhimurium, a gram positive bacteria, a purple bacteria, a green sulfur bacteria, a green non-sulfur bacteria, a cyanobacteria, a spirochetes, a thermatogale, a flavobacteria, bacteroides; a fungal cell, such as Saccharomyces cerevisiae, Pichia pastoris, and Neurospora crassa; an insect cell such as Drosophila and Spodoptera frugiperda; a mammalian cell such as CHO, COS, BHK, HEK 293 or Bowes melanoma, archaebacteria (i.e., Korarchaeota, Thermoproteus, Pyrodictium, Thermococcales, Methanogens, Archaeoglobus, and extreme Halophiles) and others.

### D. Host-Induced and Spray-Induced Gene Silencing

In one embodiment, the invention provides methods of improving HIGS of plant pests. In one embodiment, the invention provides methods of improving SIGS of plant pests. These methods may be improved by the use of Hsc70-4 polypeptides, variants, or fragments thereof in combination with dsRNA. A method of the invention comprises introduction of an interfering RNA molecule comprising a double-stranded RNA (dsRNA) or its modified forms such as small interfering RNA (siRNA) sequences, into the transgenic cells of the invention, or into their extracellular environment, such as the midgut, within a (insect) pest body wherein the dsRNA or siRNA enters the cells and inhibits expression of at least one or more target genes and wherein inhibition of the one or more target genes exerts a deleterious effect upon the (insect) pest. The interfering RNA molecule is non-naturally occurring. It is specifically contemplated that the methods and compositions of the invention will be useful in limiting or eliminating (insect) pest infestation in or on any plant by providing in the diet of the pest one or more compositions comprising the polypeptide or polynucleotide of the invention, and optionally interfering RNA molecules comprising dsRNA or siRNA molecules. The composition of the invention may also contain or be combined with interfering RNA molecules that, when delivered to a (insect) pest, inhibits through a toxic effect the ability of the (insect) pest to survive, grow, feed and/or reproduce, or to limit pest related feeding damage or loss to crop plants. It is also possible to inhibit a gene of the pest, so that the plant finally becomes tolerant to the pest infection, once it occurred.

According to an aspect of the present invention, there is provided a method of generating a pest tolerant or resistant plant, the method comprising producing a dsRNA molecule capable of silencing a pest gene in a plant cell modified to express the polypeptide of the invention.

Such dsRNA molecules can be mobile and transferred among cells and tissues; hence can occur outside cells once produced in cells. Furthermore, such dsRNA molecules can be transferred between organisms through ingestion of material derived from the dsRNA-expressing host (e.g. plant leaves and stems).

In or against plant-parasitic nematodes, e.g. root-knot nematodes, one can use for example in the transgenic cells of the invention a dsRNA that target the Meloidogyne incognita calreticulin (Mi-CRT; accession AF402771.1) transcripts as described in Rosso et al. 2005⁵⁹. Mi-CRT plays an important role in nematode infection success, because Mi-CRT knockdown by RNA interference affected the ability of the nematodes to infect plants⁶⁰.

In particular, the dsRNA useful in the methods of the invention can be synthetized by using the sequence of SEQ ID NO:109.

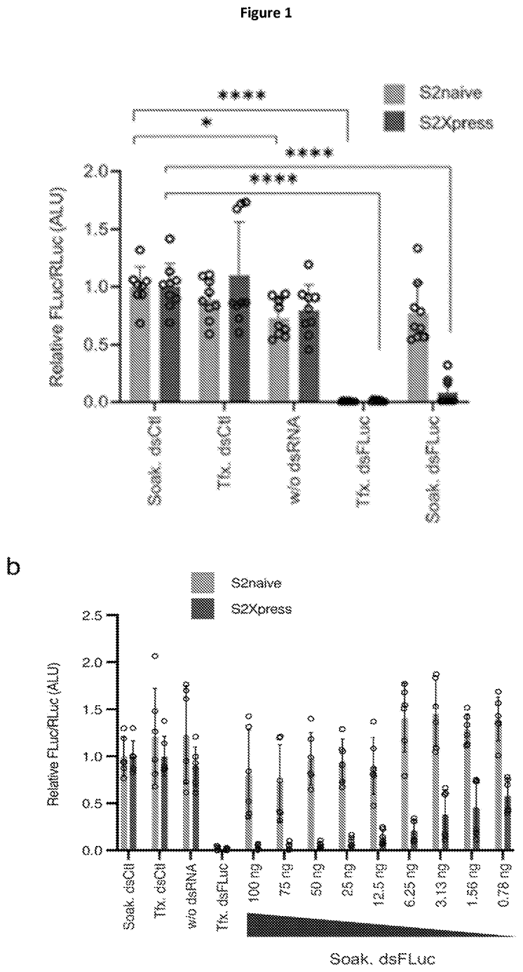

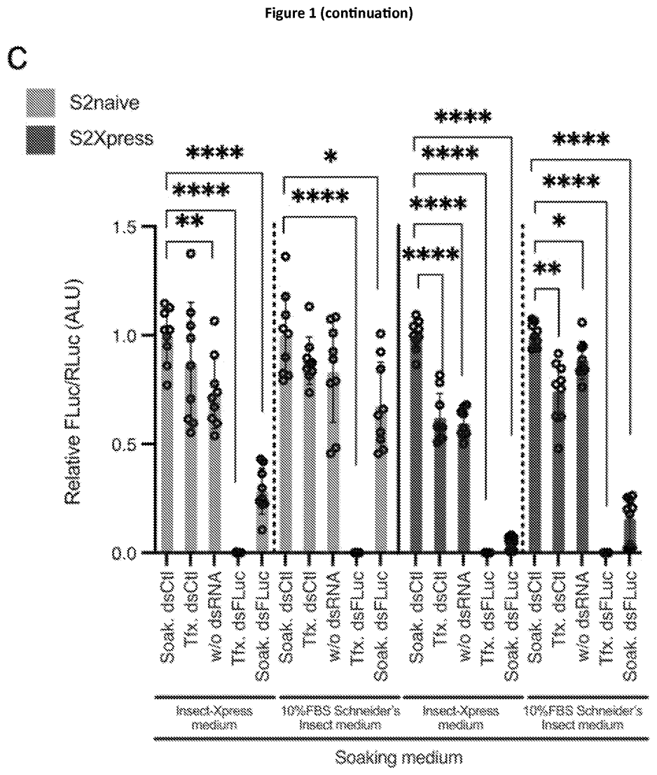

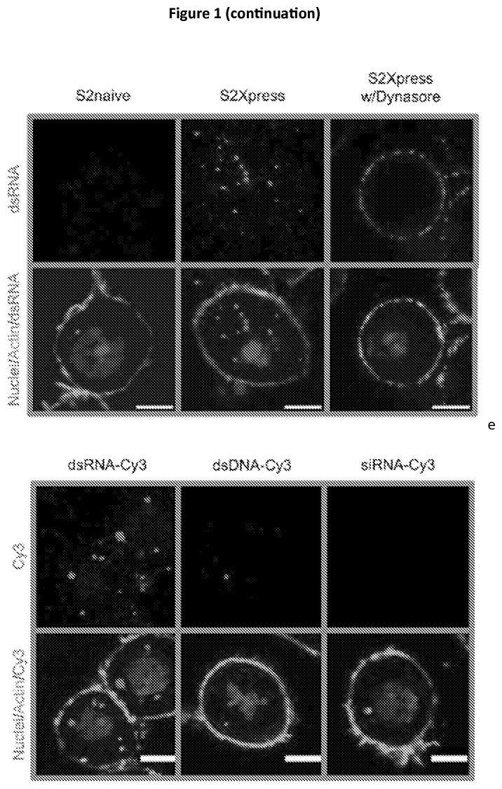

In or against aphid, in particular pea aphid Acyrthosiphon pisum, one can use a dsRNA that target the Cathepsin I Ap-cath-L/Ctsl; accession NM_001163097) transcripts as described in Jaubert-Possamai et al. 2007⁶¹. Useful dsRNAs are indicated in the FIG. 1 of this article. Silencing of cathepsin should lead to a reduction of aphid growth and fecundity as observed in Myzus persicae when the cathepsin activities is inhibited.

In particular, the dsRNA useful in the methods of the invention can be synthetized by using the sequence of SEQ ID NO:110.

As explained below, the skilled person well knows how to design and create efficient dsRNAs impairing the expression of target genes once they are identified.

The delivery of said dsRNA may be through production of the interfering RNA in a transgenic plant, for example corn, soybean, canola, rice, wheat and the like, or by topically applying a composition comprising the interfering RNA to a plant or plant seed, such as a corn plant or seed, or a soybean plant or seed, or a canola plant or seed, or a rice plant or seed, or a wheat plant or seed, and so forth. Delivery may further be through contacting the (insect) pest with the interfering RNA, such as when the (insect) pest feeds on plant material comprising the interfering RNA, either because the plant material is expressing the interfering RNA through a transgenic approach, or because the plant material is coated with a composition comprising the interfering RNA. The interfering RNA may also be provided in an artificial (insect) pest diet which the (insect) pest then contacts by feeding. The interfering RNA molecule useful in the methods or compositions of the invention comprises a nucleotide sequence that is complementary to a nucleotide sequence of a mRNA transcribable from a target gene or a portion of a nucleotide sequence of a mRNA transcribable from a target gene of the (insect) pest and therefore inhibits expression of the target gene, which causes cessation of feeding, growth, development, reproduction and eventually results in death of the (insect) pest. The nucleic acid constructs, nucleic acid molecules and recombinant vectors useful in the methods or compositions of the invention can comprise or encode at least a fragment of one strand of an interfering RNA molecule.

In one embodiment, the plant is genetically modified to express Hsc70-4. In one embodiment, the Hsc70-4 protein is sprayed on the plant. In one embodiment, an effective pest-controlling amount of the Hsc70-4 protein is sprayed on the plant together with the dsRNA. “Effective pest-controlling amount” means that concentration of a toxic protein that inhibits, through a toxic effect, the ability of pests to survive, grow, feed or reproduce, or limits pest-related damage or loss in crop plants or protects the yield potential of a crop when grown in the presence of pests. An “pest-controlling” protein, fragment, or variant thereof, is a polypeptide that inhibits, through a toxic effect, the ability of pests to survive, grow, feed or reproduce, or limits pest-related damage or loss in crop plants or protects the yield potential of a crop when grown in the presence of pests. To “deliver” a composition or toxic protein means that the composition or toxic protein comes in contact with a pest, which facilitates the oral ingestion of the composition or toxic protein, resulting in a toxic effect and control of the pest. The composition or toxic protein can be delivered in many recognized ways, including but not limited to, transgenic plant expression, formulated protein composition(s), sprayable protein composition(s), a bait matrix, or any other art-recognized protein delivery system. “Effective pest controlling amount” may or may not mean killing the pest, although it preferably means killing the pest.

In one embodiment, an effective insect-controlling amount of the Hsc70-4 protein is sprayed on the plant together with the dsRNA. “Effective insect-controlling amount” means that concentration of a toxic protein that inhibits, through a toxic effect, the ability of insects to survive, grow, feed or reproduce, or limits insect-related damage or loss in crop plants or protects the yield potential of a crop when grown in the presence of insect pests. An “insect-controlling” protein, fragment, or variant thereof, is a polypeptide that inhibits, through a toxic effect, the ability of insects to survive, grow, feed or reproduce, or limits insect-related damage or loss in crop plants or protects the yield potential of a crop when grown in the presence of insect pests. To “deliver” a composition or toxic protein means that the composition or toxic protein comes in contact with an insect, which facilitates the oral ingestion of the composition or toxic protein, resulting in a toxic effect and control of the insect. The composition or toxic protein can be delivered in many recognized ways, including but not limited to, transgenic plant expression, formulated protein composition(s), sprayable protein composition(s), a bait matrix, or any other art-recognized protein delivery system. “Effective insect controlling amount” may or may not mean killing the insects, although it preferably means killing the insects.

As used herein, “dsRNA” or “RNAi” refers to a polyribonucleotide structure formed either by a single self-complementary RNA strand or at least by two complementary RNA strands. In some embodiments, the term “dsRNA molecule” refers to double-stranded sequences of polyribonucleic acids having a first strand (sense strand) and a second strand that is a reverse complement of the first strand (anti-sense strand), the polyribonucleic acids held together by base pairing (e.g., two sequences that are the reverse complement of each other in the region of base pairing), wherein the double stranded polyribonucleic acid can be a substrate for an enzyme from the Dicer family, typically wherein the dsRNA molecule is at least 26 bp or longer. The two strands can be of identical length or of different lengths provided there is enough sequence homology between the two strands that a stable double stranded structure is formed with at least 80%, 85%, 90%, 95%, 97%, 99% or 100% complementarity over the entire length.

The degree of complementary, in other words the % identity, need not necessarily be 100%. Rather, it must be sufficient to allow the formation of a double-stranded structure under the conditions employed. As used herein, the term “fully complementary” means that all the bases of the nucleotide sequence of the dsRNA are complementary to or ‘match’ the bases of the target nucleotide sequence. The term “at least partially complementary” means that there is less than a 100% match between the bases of the dsRNA and the bases of the target nucleotide sequence. The skilled person will understand that the dsRNA need only be at least partially complementary to the target nucleotide sequence in order to mediate down-regulation of expression of the target gene. It is known in the art that RNA sequences with insertions, deletions and mismatches relative to the target sequence can still be effective at RNAi. According to the current invention, it is preferred that the dsRNA and the target nucleotide sequence of the target gene share at least 80% or 85% sequence identity, preferably at least 90% or 95% sequence identity, or more preferably at least 97% or 98% sequence identity and still more preferably at least 99% sequence identity. Alternatively, the dsRNA may comprise 1, 2 or 3 mismatches as compared with the target nucleotide sequence over every length of 24 partially complementary nucleotides. It will be appreciated by the person skilled in the art that the degree of complementarity shared between the dsRNA and the target nucleotide sequence may vary depending on the target gene to be down-regulated or depending on the (insect) pest species in which gene expression is to be controlled. It will be appreciated that the dsRNA may comprise or consist of a region of double-stranded RNA comprising annealed complementary strands, one strand of which, the sense strand, comprises a sequence of nucleotides at least partially complementary to a target nucleotide sequence within a target gene. The target nucleotide sequence may be selected from any suitable region or nucleotide sequence of the target gene or RNA transcript thereof. For example, the target nucleotide sequence may be located within the 5′UTR or 3′UTR of the target gene or RNA transcript or within exonic or intronic regions of the gene. The skilled person will be aware of methods of identifying the most suitable target nucleotide sequences within the context of the full-length target gene. For example, multiple dsRNAs targeting different regions of the target gene can be synthesised and tested. Alternatively, digestion of the RNA transcript with enzymes such as RNAse H can be used to determine sites on the RNA that are in a conformation susceptible to gene silencing. Target sites may also be identified using in silico approaches, for example, the use of computer algorithms designed to predict the efficacy of gene silencing based on targeting different sites within the full-length gene. Preferably, the % identity of a polyribonucleotide is determined by GAP (Needleman and Wunsch, 1970) analysis (GCG program) using the default settings, wherein the query sequence is at least about 21 to about 23 nucleotides in length, and the GAP analysis aligns the two sequences over a region of at least about 21 nucleotides. In another embodiment, the query sequence is at least 150 nucleotides in length, and the GAP analysis aligns the two sequences over a region of at least 150 nucleotides. In a further embodiment, the query sequence is at least 300 nucleotides in length and the GAP analysis aligns the two sequences over a region of at least 300 nucleotides. In yet another embodiment, the query sequence corresponds to the full length of the target RNA, for example mRNA, and the GAP analysis aligns the two sequences over the full length of the target RNA.

Conveniently, the dsRNA can be produced from a single open reading frame in a recombinant host cell, wherein the sense and anti-sense sequences are flanked by an unrelated sequence which enables the sense and anti-sense sequences to hybridize to form the dsRNA molecule with the unrelated sequence forming a loop structure. Alternatively, the sense strand and antisense strand can be made without an open reading frame to ensure that no protein will be made in the transgenic host cell. The two strands can also be expressed separately as two transcripts, one encoding the sense strand and one encoding the antisense strand. RNA duplex formation can be initiated either inside or outside the cell. The dsRNA can be partially or fully double-stranded. The RNA can be enzymatically or chemically synthesized, either in vitro or in vivo. The dsRNA need not be full length relative to either the primary transcription product or fully processed RNA. It is well-known in the art that small dsRNA of about 19-23 bp in length can be used to trigger gene silencing of a target gene. Generally, higher identity can be used to compensate for the use of a shorter sequence. Furthermore, the dsRNA can comprise single stranded regions as well, e.g., the dsRNA can be partially or fully double stranded. The double stranded region of the dsRNA can have a length of at least about 19 to about 23 base pairs, optionally a sequence of about 19 to about 50 base pairs, optionally a sequence of about 50 to about 100 base pairs, optionally a sequence of about 100 to about 200 base pairs, optionally a sequence of about 200 to about 500, and optionally a sequence of about 500 to about 1000 or more base pairs, up to a molecule that is double stranded for its full length, corresponding in size to a full length target RNA molecule. Bolognesi et al (2012, PLOS One, 7 (10): e47534, herein incorporated by reference) teach that dsRNAs greater than or equal to about 60 bp are required for biological activity in artificial diet bioassays with Southern Com Rootworm (SCR; Diabrotica undecimpunctata howardii). Mao et al (2007, Nature Biotechnology, 35 (11): 1307-1313) teach a transgenic plant expressing a dsRNA construct against a target gene (CYP6AE14) of an (insect) pest (cotton bollworm, Helicoverpa armigera). Insects feeding on the transgenic plant have small RNAs of about 19-23 bp in size of the target gene in their midgut, with a corresponding reduction in CYP6AE14 transcripts and protein. This suggests that the small RNAs were efficacious in reducing expression of the target gene in the (insect) pest. Therefore, small RNAs of about 19 bp, about 20 bp, about 21 bp, about 22 bp, about 23 bp, about 24 bp, about 25 bp, about 26 bp, about 27 bp, about 28 bp, about 29 bp, or about 30 bp may be efficacious in reducing expression of the target gene in an (insect) pest. Alternatively, the dsRNA may comprise a target dsRNA of at least 19 base pairs, and the target dsRNA may be within a dsRNA “carrier” or “filler” sequence. For example, Bolognesi et al (2012) show that a 240 bp dsRNA encompassing a target dsRNA, which comprised a 21 bp consecutive sequence with 100% identity to the target sequence, had biological activity in bioassays with Southern Com Rootworm. The present application exemplifies a similar approach in bioassays with Western Com Rootworm. The target dsRNA may have a length of at least 19 to about 25 base pairs, optionally a sequence of about 19 to about 50 base pairs, optionally a sequence of about 50 to about 100 base pairs, optionally a sequence of about 100 to about 200 base pairs, optionally a sequence of about 200 to about 500, and optionally a sequence of about 500 to about 1000 or more base pairs. Combined with the carrier dsRNA sequence, the dsRNA of the target sequence and the carrier dsRNA may have a total length of at least about 50 to about 100 base pairs, optionally a sequence of about 100 to about 200 base pairs, optionally a sequence of about 200 to about 500, and optionally a sequence of about 500 to about 1000 or more base pairs. The dsRNA can contain known nucleotide analogs or modified backbone residues or linkages, which are synthetic, naturally occurring, and non-naturally occurring. Examples of such analogs include, without limitation, phosphorothioates, phosphoramidates, methyl phosphonates, chiralmethyl phosphonates and 2-O-methyl ribonucleotides.

By use of the term “complementation”, “complementarity” or “complementary” is meant that the RNA molecules (or at least a portion of it that is present in the processed small RNA form, or at least one strand of a double-stranded polynucleotide or portion thereof, or a portion of a single strand polynucleotide) hybridizes under physiological conditions to the target RNA (e.g. transcript of the plant gene), or a fragment thereof, to effect regulation or function of Hsc70-4. For example, in some embodiments, a RNA molecule (e.g. small RNA molecule) has 100% sequence identity or at least about 30, 40, 45, 50, 55, 60, 65, 70, 75, 80, 83, 84, 85, 86, 87, 88, 89, 90, 91, 92, 93, 94, 95, 96, 97, 98, or 99% sequence identity when compared to a sequence of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, 100, 150, 200, 300, 400, 500 or more contiguous nucleotides in the target RNA (or family members of a given target gene).

As used herein, an RNA molecule, or its processed small RNA forms (discussed in further detail hereinbelow), are said to exhibit “complete complementarity” when every nucleotide of one of the sequences read 5′ to 3′ is complementary to every nucleotide of the other sequence when read 3′ to 5′. A nucleotide sequence that is completely complementary to a reference nucleotide sequence will exhibit a sequence identical to the reverse complement sequence of the reference nucleotide sequence. Methods for determining sequence complementarity are well known in the art and include, but are not limited to, bioinformatics tools which are well known in the art (e.g. BLAST, multiple sequence alignment).

According to one embodiment, the dsRNA molecule is longer than 20 bp, which may be called a “long dsRNA molecule.” According to one embodiment, the dsRNA molecule is longer than 21 bp. According to one embodiment, the dsRNA molecule is longer than 22 bp. According to one embodiment, the dsRNA molecule is longer than 23 bp. According to one embodiment, the dsRNA molecule is longer than 24 bp. According to one embodiment, the dsRNA molecule comprises 20-100,000 bp. According to one embodiment, the dsRNA molecule comprises 20-10,000 bp. According to one embodiment, the dsRNA molecule comprises 20-1,000 bp.

According to one embodiment, the dsRNA molecule comprises 20-500 bp. According to one embodiment, the dsRNA molecule comprises 20-50 bp. According to one embodiment, the dsRNA molecules comprise 200-5000 bp. According to one embodiment, the dsRNA molecules comprise 200-1000 bp. According to one embodiment, the dsRNA molecules comprise 200-500 bp. According to one embodiment, the dsRNA molecules comprise 2000-100,000 bp. According to one embodiment, the dsRNA molecules comprise 2000-10,000 bp. According to one embodiment, the dsRNA molecules comprise 2000-5000 bp. According to one embodiment, the dsRNA molecules comprise 10,000-100,000 bp. According to one embodiment, the dsRNA molecules comprise 1,000-10,000 bp. According to one embodiment, the dsRNA molecules comprise 100-10,000 bp. According to one embodiment, the dsRNA molecules comprise 100-1,000 bp. According to one embodiment, the dsRNA molecules comprise 10-1,000 bp. According to one embodiment, the dsRNA molecules comprise 10-100 bp. According to one embodiment, the dsRNA molecule comprises an overhang, i.e. a non-double stranded region of a dsRNA molecule (i.e., single stranded RNA). According to one embodiment, the dsRNA molecule does not comprise an overhang.

According to one embodiment, the dsRNA molecule useful in the methods or compositions of the invention can be processed into small RNA molecules capable of engaging with RNA-induced silencing complex (RISC). Accordingly, this dsRNA molecule may serve as a substrate for the intracellular RNAi processing machinery (i.e. may be a precursor RNA molecule) and may be processed by ribonucleases, including but not limited to, the DICER protein family (e.g. DCR1 and DCR2), DICER-LIKE protein family (e.g. DCL1, DCL2, DCL3, DCL4), ARGONAUTE protein family (e.g. AGOI, AG02, AG03, AG04), tRNA cleavage enzymes (e.g. RNY1, ANGIOGENIN, RNase P, RNase P-like, SLFN3, ELAC1 and ELAC2), and Piwi-interacting RNA (piRNA) related proteins (e.g. AG03, AUBERGINE, HIWI, HIWI2, HIW13, PIWI), ALG1 and ALG2) into small RNA molecules.

The term “plant” as used herein encompasses whole plants, a grafted plant, ancestors and progeny of the plants and plant parts, including seeds, shoots, stems, roots (including tubers), rootstock, scion, and plant cells, tissues and organs. The plant may be in any form including suspension cultures, embryos, meristematic regions, callus tissue, leaves, gametophytes, sporophytes, pollen, and microspores. Plants that may be useful in the methods of the invention include all plants which belong to the superfamily Viridiplantee, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Cannabaceae, Cannabis indica, Ccamabis, Cannabis sativa, Hemp, industrial Hemp, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalyptus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparriienia rufa, Hypericum erectum, Hypeffhelia dissolute, Indigo incamata, Iris spp., Leptarrhena pyrolifolia, Lespediza spp, Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, banana, Nicotianum spp., Onobrychis spp., Omithopus spp., Oryza spp., Peltophorum africanum, Pennisetum spp., Persea gratissima, Petunia spp., Phaseolus spp., Phoenix canadensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellate, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively, algae and other non-Viridiplantae can be used for the methods of some embodiments of the invention.

In one embodiment, the plants of interest include grain plants that provide seeds of interest, oilseed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc

According to a specific embodiment, the plant is a crop, a flower, a weed or a tree. According to a specific embodiment, the plant is a woody plant species e.g., Actinidia chinensis (Actinidiaceae), Manihotesculenta (Euphorbiaceae), Firiodendron tulipifera (Magnoliaceae), Populus (Salicaceae), Santalum album (Santalaceae), Ulmus (Ulmaceae) and different species of the Rosaceae (Malus, Prunus, Pyrus) and the Rutaceae (Citrus, Microcitrus), Gymnospermae e.g., Picea glauca and Pinus taeda, forest trees (e.g., Betulaceae, Fagaceae, Gymnospermae and tropical tree species), fruit trees, shrubs or herbs, e.g., (banana, cocoa, coconut, coffee, date, grape and tea) and oil palm. According to a specific embodiment, the plant is of a tropical crop e.g., coffee, macadamia, banana, pineapple, taro, papaya, mango, barley, beans, cassava, chickpea, cocoa (chocolate), cowpea, maize (com), millet, rice, sorghum, sugarcane, sweet potato, tobacco, taro, tea, yam.

“Grain,” “seed,” or “bean,” refers to a flowering plant's unit of reproduction, capable of developing into another such plant. As used herein, the terms are used synonymously and interchangeably. According to a specific embodiment, the plant is a plant cell e.g., plant cell in an embryonic cell suspension. According to a specific embodiment, the plant cell is a protoplast. protoplasts are derived from any plant tissue e.g., fruit, flowers, roots, leaves, embryos, embryonic cell suspension, calli or seedling tissue. According to a specific embodiment, the plant cell is an embryogenic cell. According to a specific embodiment, the plant cell is a somatic embryogenic cell.

As used herein the term “pest” refers to an organism which directly or indirectly harms the plant. A direct effect includes, for example, feeding on the plant leaves. Indirect effect includes, for example, transmission of a disease agent (e.g. a virus, bacteria, etc.) to the plant. In the latter case the pest serves as a vector for pathogen transmission.

According to some embodiments, a pest is an invertebrate pest, including an invertebrate pest which is susceptible to dsRNA via methods such as, but not limited to, ingestion and/or soaking. Each possibility represents a separate embodiment of the present invention. According to some embodiment, an invertebrate pest which is susceptible to dsRNA is susceptible to dsRNA of 26 bp and above, possibly of about 26-50 bp. Each possibility represents a separate embodiment of the present invention. According to one embodiment, the pest is an invertebrate organism. Exemplary pests include, but are not limited to, insects, nematodes, snails, slugs, spiders, caterpillars, scorpions, mites, ticks, fungi, and the like. Insect pests include, but are not limited to, insects selected from the orders Coleoptera (e.g. beetles), Diptera (e.g. flies, mosquitoes), Hymenoptera (e.g. sawflies, wasps, bees, and ants), Lepidoptera (e.g. butterflies and moths), Mallophaga (e.g. lice, e.g. chewing lice, biting lice and bird lice), Hemiptera (e.g. true bugs), Homoptera including suborders Stemorrhyncha (e.g. aphids, whiteflies, and scale insects), Auchenorrhyncha (e.g. cicadas, leafhoppers, treehoppers, planthoppers, and spittlebugs), and Coleorrhyncha (e.g. moss bugs and beetle bugs), Orthroptera (e.g. grasshoppers, locusts and crickets, including katydids and wetas), Thysanoptera (e.g. Thrips), Dermaptera (e.g. Earwigs), Isoptera (e.g. Termites), Anoplura (e.g. Sucking lice), Siphonaptera (e.g. Flea), Trichoptera (e.g. caddisflies), etc.

Insect pests affected by the compositions and methods of the invention include, but are not limited to:
Maize: Ostrinia nubilalis, European com borer; Agrotis ipsilon, black cutworm; Helicoverpa zea, com earworm; Spodoptera frugiperda, fall armyworm; Diatraea grandiosella, southwestern com borer; Elasmopalpus lignosellus, lesser cornstalk borer; Diatraea saccharalis, surgarcane borer; Diabrotica virgifera, western com rootworm; Diabrotica longicomis barberi, northern corn rootworm; Diabrotica undecimpunctata howardi, southern com rootworm; Melanotus spp., wireworms; Cyclocephala borealis, norther masked chafer (white gmb); Cyclocephala immaculata, southern masked chafer (white gmb); Popillia japonica, Japanese beetle; Chaetocnema pulicaria, com flea beetle; Sphenophoms maidis, maize billbug; Rhopalosiphum maidis, com leaf aphid; Anuraphis maidiradicis, com root aphid; Blissus leucoptems leucoptems, chinch bug; Melanoplus femurrubrum, redlegged grasshopper; Melanoplus sanguinipes, migratory grasshopper; Hylemya platura, seedcom maggot; Agromyza parvicomis, com blot leafminer; Anaphothrips obscrums, grass thrips; Solenopsis milesta, thief ant; Tetranychus urticae, twospotted spider mite;
Sorghum: Chilo partellus, sorghum borer; Spodoptera frugiperda, fall armyworm; Helicoverpa zea, com earworm; Elasmopalpus lignosellus, lesser cornstalk borer; Feltia subterranea, granulate cutworm; Phyllophaga crinita, white gmb; Eleodes, Conodems, and Aeolus spp., wireworms; Oulema melanopus, cereal leaf beetle; Chaetocnema pulicaria, com flea beetle; Sphenophoms maidis, maize billbug; Rhopalosiphum maidis; com leaf aphid; Sipha flava, yellow sugarcane aphid; Blissus leucoptems leucoptems, chinch bug; Contarinia sorghicola, sorghum midge; Tetranychus cinnabarinus, carmine spider mite; Tetranychus urticae, twospotted spider mite;
Wheat: Pseudaletia unipunctata, army worm; Spodoptera frugiperda, fall armyworm; Elasmopalpus lignosellus, lesser cornstalk borer; Agrotis orthogonia, western cutworm; Elasmopalpus lignosellus, lesser cornstalk borer; Oulema melanopus, cereal leaf beetle; Hypera punctata, clover leaf weevil; Diabrotica undecimpunctata howardi, southern com rootworm; Russian wheat aphid; Schizaphis graminum, greenbug; Macrosiphum avenae, English grain aphid; Melanoplus femurrubrum, redlegged grasshopper; Melanoplus differentialis, differential grasshopper; Melanoplus sanguinipes, migratory grasshopper; Mayetiola destructor, Hessian fly; Sitodiplosis mosellana, wheat midge; Meromyza americana, wheat stem maggot; Hylemya coarctate, wheat bulb fly; Frankliniella fusca, tobacco thrips; Cephus cinctus, wheat stem sawfly; Aceria tulipae wheat curl mite;
Sunflower: Suleima helianthana, sunflower bud moth; Homoeosoma electellum, sunflower moth; zygogramma exclamationis, sunflower beetle; Bothyrus gibbosus, carrot beetle; Neolasioptera murtfeldtiana, sunflower seed midge;
Cotton: Heliothis virescens, cotton budworm; Helicoverpa zea, cotton bollworm; Spodoptera exigua, beet armyworm; Pectinophora gossypiella, pink bollworm; Anthonomus grandis, boll weevil; Aphis gossypii, cotton aphid; Pseudatomoscelis seriatus, cotton fleahopper; Trialeurodes abutilonea, bandedwinged whitefly; Lygus lineolaris, tarnished plant bug; Melanoplus femurrubrum, redlegged grasshopper; Melanoplus differentalis, differential grasshopper; Thrips tabaci, onion thrips; Franklinkiella fusca, tobacco thrips; Tetranychus cinnabarinus, carmine spider mite; Tetranychus urticae, twospotted spider mite;
Rice: Diatraea saccharalis, sugarcane borer; Spodoptera frugiperda, fall armyworm; Helicoverpa zea, com earworm; Colaspis brunnea, grape colaspis; Lissorhoptms oryzophilus, rice water weevil; Sitophilus oryzae, rice weevil; Nephotettix nigropictus, rice leafhopper; Blissus leucopterus leucoptems, chinch bug; Acrostemum hilare, green stink bug;
Soybean: Pseudoplusia includens, soybean looper; Anticarsia gemmatalis, velvetbean caterpillar; Plathypena scabs, green cloverworm; Ostrinia nubilalis, European com borer; Agrotis ipsilon, black cutworm; Spodoptera exigua, beet armyworm; Heliothis virescens, cotton budworm; Helicoverpa zea, cotton bollworm; Epilachna varivestis, Mexican bean beetle; Myzus persicae, green peach aphid; Empoasca fabae, potato leafhopper; Acrostemum hilare, green stink bug; Melanoplus femurrubrum, redlegged grasshopper; Melanoplus differentialis, differential grasshopper; Hylemya platura, seedcom maggot; Sericothrips variabilis, soybean thrips; Thrips tabaci, onion thrips; Tetranychus turkestani, strawberry spider mite; Tetranychus urticae, twospotted spider mite;
Barley: Ostrinia nubilalis, European com borer; Agrotis ipsilon, black cutworm; Schizaphis graminum, greenbug; Blissus leucoptems leucoptems, chinch bug; Acrostemum hilare, green stink bug; Euschistus servus, brown stink bug; Delia platura, seedcom maggot; Mayetiola destmctor, Hessian fly; Petrobia latens, brown wheat mite;
Oil Seed Rape: Brevicoryne brassicae, cabbage aphid; Phyllotreta cmciferae, Flea beetle; Mamestra configurata, Bertha armyworm; Plutella xylostella, Diamond-back moth; Delia ssp., Root maggots.

More particularly, the invention encompasses compositions and methods of modulating expression of one or more target genes in coleopteran insect pests, such as Sitophilus oryzae (So; rice weevil), Diabrotica virgifera virgifera (Dv; western com rootworm), Diabrotica undecimpunctata howardi (Du; southern com rootworm), Diabrotica barberi (Db; northern com rootworm), Phyllotreta armoraciae (Pa horseradish flea beetle), Phyllotreta nemorum (Pn; turnip flea beetle), Phyllotreta cruciferae (Pu; cmcifer flea beetle), Phyllotreta striolata (Ps; striped flea beetle), Phyllotreta atra (Pt), Psylliodes chrysocephala (Pc; cabbage-stem flea beetle), Meligethes aeneus (Ma; pollen beetle), Ceutorhynchus assimilis (Ca; cabbage seedpod weevil), Leptinotarsa decemlineata (Ld; Colorado potato beetle), and/or in hemipteran insect pests, such as, Nezara viridula (Nv; green stink bug), Euschistus herns (Eh; brown stink bug), and Piezodorus guildinii (Pg; red-banded stink bug), and related species, that causes cessation of feeding, growth, development and reproduction, and eventually results in the death of the insect.

The invention also provides a method of controlling a Lepidoptera, coleopteran and/or hemipteran insect plant pest comprising contacting the coleopteran and/or hemipteran insect pest with a composition of the invention as well as with a nucleic acid molecule that is capable of producing an interfering RNA for inhibiting expression of a gene in the coleopteran and/or hemipteran insect pest thereby controlling the coleopteran and/or hemipteran insect pest. In some aspects, the coleopteran insect pest is selected from the group consisting of the coleopteran insect pest is selected from the group consisting of Helicoverpa armigera, Sitophilus oryzae (So; rice weevil), Diabrotica virgifera virgifera (Dv; western corn rootworm), Diabrotica undecimpunctata howardi (Du; southern corn rootworm), Diabrotica barberi (Db northern corn rootworm), Phyllotreta armoraciae (Pa; horseradish flea beetle) Phyllotreta nemorum (Pn; turnip flea beetle), Phyllotreta cruciferae (Pu; crucifer flea beetle), Phyllotreta striolata (Ps; striped flea beetle), Phyllotreta atra (Pt), Psylliodes chrysocephala (Pc; cabbage-stem flea beetle), Meligethes aeneus (Ma; pollen beetle), Ceutorhynchus assimilis (Ca; cabbage seedpod weevil), Leptinotarsa decemlineata (Ld; Colorado potato beetle), and/or the hemipteran insect pest is selected from the group consisting of Nezara viridula (Nv; green stink bug), Euschistus heros (Eh; brown stink bug), and Piezodorus guildinii (Pg; red-banded stink bug). In some embodiments, the insect gene is selected from homeodomain transcription factors, chitin deacetylase, Cytochrome P450 enzyme system, HMG-Coa reductase, Juvenile hormone esterase related gene, tubulin, chitinase, ARF1, carboxypeptidase, ATPase, helicase, Period clock gene, chitin synthase gene A (SeCHSA), Pigmentation causing gene, Hemolin, Bt toxin receptor, Acetylcholinesterase (AChE) and ecdysone receptor (EcR) homologous genes of B. tabaci, Juvenile hormone-binding protein (JHBP) and vacuolar ATPase subunit H (V-ATPase-H), DvSnf7, ATPase, chromatin remodelling genes which affects the sperm viability, Leg gene, a Tribolium homologue of proboscipedia (pb) and homeotic genes, β-actin and shrub proteins, dsRNA against sclerotization gene led to death of larva. A more complete list can be found in Nitnavare R B, Bhattacharya J, Singh S, Kour A, Hawkesford M J, Arora N. Next Generation dsRNA-Based Insect Control: Success So Far and Challenges. Front Plant Sci. 2021 Oct. 18; 12:673576. doi: 10.3389/fpls.2021.673576. PMID: 34733295; PMCID: PMC8558349.

The phrase “silencing a pest gene” refers to reducing the level of expression of a polynucleotide or the polypeptide encoded thereby, by at least about 10%, 20%, 30%, 40%, 50%, 60%, 70%, 80%, 90%, 95%, 99% or by 100%, as compared to a pest gene not targeted by the designed dsRNA molecule.

Preferably, silencing of the pest gene results in the suppression, control, and/or killing of the pest which results in limiting the damage that the pest causes to the plant. Controlling a pest includes, but is not limited to, killing the pest, inhibiting development of the pest, altering fertility or growth of the pest in such a manner that the pest provides less damage to the plant, decreasing the number of offspring produced, producing less fit pests, producing pests more susceptible to predator attack, or deterring the pests from eating the plant. According to one embodiment, silencing a pest gene reduces disease symptoms in a plant or reduces damage to the plant (resulting from the pest) by at least about 10%, 20%, 30%, 40%, 50%, 60%, 70%, 80%, 90%, 95%, 99% or by 100%, as compared to a plant harmed by the pest and not being subjected to the designed dsRNA molecule. Assays measuring the control of a pest are commonly known in the art, see, for example, U.S. Pat. No. 5,614,395, herein incorporated by reference. Such techniques include, measuring over time, the average lesion diameter, the pathogen biomass, and the overall percentage of decayed plant tissues. See, for example, Thomma et al. (1998) Plant Biology 95:15107-15111, herein incorporated by reference. See, also Baum et al. (2007) Nature Biotech 11:1322-1326 and WO 2007/035650 which provide both whole plant feeding assays and com root feeding assays.

“Expression cassette” as used herein means a nucleic acid molecule capable of directing expression of at least one polynucleotide of interest, such as a polynucleotide that encodes an interfering RNA molecule and/or a Hsc70-4 protein of the invention, in an appropriate host cell, comprising a promoter operably linked to the polynucleotide of interest a Hsc70-4 protein of the invention which is operably linked to a termination signal. An “expression cassette” also typically comprises additional polynucleotides required for proper translation of the polynucleotide of interest. The expression cassette may also comprise other polynucleotides not necessary in the direct expression of a polynucleotide of interest but which are present due to convenient restriction sites for removal of the cassette from an expression vector. The expression cassette comprising the polynucleotide(s) of interest may be chimeric, meaning that at least one of its components is heterologous with respect to at least one of its other components. The expression cassette may also be one that is naturally occurring but has been obtained in a recombinant form useful for heterologous expression. Typically, however, the expression cassette is heterologous with respect to the host, i.e. the polynucleotide of interest in the expression cassette does not occur naturally in the host cell and must have been introduced into the host cell or an ancestor of the host cell by a transformation process or a breeding process. The expression of the polynucleotide(s) of interest in the expression cassette is generally under the control of a promoter. In the case of a multicellular organism, such as a plant, the promoter can also be specific or preferential to a particular tissue, or organ, or stage of development. An expression cassette, or fragment thereof, can also be referred to as “inserted polynucleotide” or “insertion polynucleotide” when transformed into a plant. Constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785 5,399,680; 5,268,463; 5,608,142; and 6,177,611. Synthetic promoters can be used to express Hsc70-4 sequences or biologically active variants and fragments thereof. Synthetic promoters include for example a combination of one or more heterologous regulatory elements.

### E. Nucleic Acid and/or Protein Administration

To “deliver” a composition of the invention (comprising an oligonucleotide the invention and optionally a dsRNA) or the Hsc70-4 protein of the invention also means that the composition or Hsc70-4 protein comes in contact with an (insect) pest, which facilitates the ingestion of the composition or Hsc70-4 protein, resulting in a toxic effect and control of the (insect) pest. The composition or Hsc70-4 protein can be delivered in many recognized ways, including but not limited to, transgenic plant expression, formulated protein composition(s), sprayable protein composition(s), a bait matrix, or any other art-recognized protein delivery system. In the context of the invention, the term “toxic” used to describe a dsRNA useful in the compositions and methods of the invention means that the dsRNA molecules and combinations of such dsRNA molecules function as orally active (insect) pest control agents that have a negative effect on an (insect) pest. When a composition of the invention is delivered to the insect, the result is typically death of the (insect) pest, or the (insect) pest does not feed upon the source that makes the composition available to the (insect) pest. Such a composition may be a transgenic plant expressing a dsRNA, preferably with the Hsc70-4 protein of the invention and an agriculturally acceptable carrier.

An “agriculturally acceptable carrier” includes adjuvants, mixers, enhancers, etc. beneficial for application of an active ingredient, such as molecules of the invention. Suitable carriers should not be phytotoxic to valuable crops, particularly at the concentrations employed in applying the compositions in the presence of crops, and should not react chemically with the compounds of the active ingredient herein, or other composition ingredients. Such mixtures can be designed for application directly to crops, or can be concentrates or formulations which are normally diluted with additional carriers and adjuvants before application. They may include inert or active components and can be solids, such as, for example, dusts, granules, water dispersible granules, or wettable powders, or liquids, such as, for example, emulsifiable concentrates, solutions, emulsions or suspensions. Suitable agricultural carriers may include liquid carriers, for example water, toluene, xylene, petroleum naphtha, crop oil, acetone, methyl ethyl ketone, cyclohexanone trichloroethylene, perchloroethylene, ethyl acetate, amyl acetate, butyl acetate, propylene glycol monomethyl ether and diethylene glycol monomethyl ether, methanol, ethanol, isopropanol, amyl alcohol, ethylene glycol, propylene glycol, glycerine, and the like. Water is generally the carrier of choice for the dilution of concentrates. Suitable solid carriers may include talc, pyrophyllite clay, silica, attapulgus clay, kieselguhr, chalk, diatomaceous earth, lime, calcium carbonate, bentonire clay, Fuller's earth, cotton seed hulls, wheat flour, soybean flour, pumice, wood flour, walnut shell flour, lignin, and the like.

For the present invention, an agriculturally acceptable carrier may also include non-pathogenic, attenuated strains of microorganisms, which carry the (insect) pest control agent, namely an interfering RNA molecule, preferably with a gene expressing Hsc70-4 protein. In this case, the microorganisms carrying the interfering RNA and/or Hsc70-4 protein may also be referred to as “(insect) pest control agents”. The microorganisms may be engineered to express a nucleotide sequence of a target gene to produce interfering RNA molecules comprising RNA sequences homologous or complementary to RNA sequences typically found within the cells of an (insect) pest and/or Hsc70-4 protein. Exposure of the (insects) pests to the microorganisms result in ingestion of the microorganisms and down-regulation of expression of target genes mediated directly or indirectly by the interfering RNA molecules or fragments or derivatives thereof and facilitated or improved by increased expression of Hsc70-4 protein.

In another embodiment, the oligonucleotide molecules of the invention may be encapsulated in a synthetic matrix such as a polymer and applied to the surface of a host such as a plant. Ingestion of the host cells by an (insect) pest permits delivery of the (insect) pest control agents to the (insect) pest and results in down-regulation of a target gene in the host.

A composition of the invention, for example a composition comprising the Hsc70-4 protein of the invention and optionally an interfering RNA molecule and an agriculturally acceptable carrier, may be used in conventional agricultural methods. For example, the compositions of the invention may be mixed with water and/or fertilizers and may be applied preemergence and/or postemergence to a desired locus by any means, such as airplane spray tanks, irrigation equipment, direct injection spray equipment, knapsack spray tanks, cattle dipping vats, farm equipment used in ground spraying (e.g., boom sprayers, hand sprayers), and the like. The desired locus may be soil, seeds, plants, and the like.

A composition of the invention may be applied to a seed or plant propagule in any physiological state, at any time between harvest of the seed and sowing of the seed; during or after sowing; and/or after sprouting. It is preferred that the seed or plant propagule be in a sufficiently durable state that it incurs no or minimal damage, including physical damage or biological damage, during the treatment process. A formulation may be applied to the seeds or plant propagules using conventional coating techniques and machines, such as fluidized bed techniques, the roller mill method, rotostatic seed treaters, and drum coaters.

In order to apply an active ingredient to (insect) pests and/or crops of useful plants as required by the methods of the invention said active ingredient may be used in pure form or, more typically, formulated into a composition which includes, in addition to said active ingredient, a suitable inert diluent or carrier and optionally, a surface active agent (SFA). SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). SFAs include non-ionic, cationic and/or anionic surfactants, as well as surfactant mixtures. Thus in further embodiments according to any aspect of the invention mentioned hereinbefore, the active ingredient will be in the form of a composition additionally comprising an agriculturally acceptable carrier or diluent.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the active ingredient.

Dustable powders (DP) may be prepared by mixing the active ingredient with one or more solid diluents (for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers) and mechanically grinding the mixture to a fine powder Soluble powders (SP) may be prepared by mixing the active ingredient with one or more water-soluble inorganic salts (such as sodium bicarbonate, sodium carbonate or magnesium sulfate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Granules (GR) may be formed either by granulating a mixture of the active ingredient and one or more powdered solid diluents or carriers, or from pre-formed blank granules by absorbing the active ingredient (or a solution thereof, in a suitable agent) in a porous granular material (such as pumice, attapulgite clays, fuller's earth, kieselguhr, diatomaceous earths or ground corn cobs) or by adsorbing the active ingredient (or a solution thereof, in a suitable agent) on to a hardcore material (such as sands, silicates, mineral carbonates, sulfates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible concentrates (DC) may be prepared by dissolving the active ingredient in water or an organic solvent, such as a ketone, alcohol or glycol ether. These solutions may contain a surface active agent (for example to improve water dilution or prevent crystallisation in a spray tank). Emulsifiable concentrates (EC) or oil-in-water emulsions (EW) may be prepared by dissolving the active ingredient in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 15060 and SOLVESSO 200; SOLVESSO is a Registered TradeMark), ketones (such as cyclohexanone or methylcyclohexanone) and alcohols (such as benzyl alcohol, furfuryl alcohol or butanol), N-alkylpyrrolidones (such as N-methylpyrrolidoneor N-octylpyrrolidone), dimethyl amides of fatty acids (such as C8-C10 fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining the active ingredient either as a liquid (if it is not a liquid at room temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. The active ingredient is present initially in either the water or the solvent/SPA blend. Suitable solvents for use in MEs include those hereinbefore described for use in ECs or in EWs. A ME may be either an oil-in-water or a water-in-oil system (which system is present may be determined by conductivity measurements) and may be suitable for mixing water-soluble and oil-soluble pesticides in the same formulation. A ME is suitable for dilution into water, either remaining as a microemulsion or forming a conventional oil-in-water emulsion.

Suspension concentrates (SC) may comprise aqueous or non-aqueous suspensions of finely divided insoluble solid particles of the active ingredient. SCs may be prepared by ball or bead milling the solid active ingredient in a suitable medium, optionally with one or more dispersing agents, to produce a fine particle suspension of the compound. One or more wetting agents may be included in the composition and a suspending agent may be included to reduce the rate at which the particles settle.

Alternatively, the active ingredient may be dry milled and added to water, containing agents hereinbefore described, to produce the desired end product.

Aerosol formulations comprise the active ingredient and a suitable propellant (for example n-butane). Active ingredients may also be dissolved or dispersed in a suitable medium (for example water or a water miscible liquid, such as n-propanol) to provide compositions for use in non-pressurized, hand-actuated spray pumps. The active ingredient may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains the active ingredient and, optionally, a carrier or diluent therefor. The polymeric shell may be produced by either an interfacial polycondensation reaction or by a coacervation procedure. The compositions may provide for controlled release of the compound of the active ingredient. Active ingredients may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of the active ingredient). Such additives include surface active agents, spray additives based on oils, for example certain mineral oils, natural plant oils (such as soy bean and rape seed oil) and/or modified plant oils (e.g. esterified plant oils), and blends of these with other bio-enhancing adjuvants (ingredients which may aid or modify the action of the active ingredient. Where the active ingredient described herein is employed in methods of protecting crops of useful plants, methods of enhancing/maintaining yield and/or methods of increasing/maintaining pollination in crops of useful plants, it is preferred that said active ingredient (or compositions containing such active ingredient) is applied to the crop of useful plants at the flower-bud stage. In particular for crops of useful plants wherein said plants have yellow flowers, (e.g. oilseed rape, mustard etc.) it is preferred that the application occurs at the green to yellow bud stage.

In some embodiments, the dsRNA, Hsc70-4 protein, and compositions comprising one or both, may be sprayed onto plant leaves, which may then spread through the rest of the plant to one or more distal areas. This is a process known as SIGS.

### F. Transgenic Plants

In some embodiments, the acceptable agricultural carrier is a transgenic organism expressing the oligonucleotides or protein of the invention. In some embodiments the transgenic organism may be a transgenic plant. This transgenic plant may be furthermore expressing an interfering RNA that when fed upon by a target coleopteran and/or hemipteran plant pest causes the target coleopteran and/or hemipteran plant pest to stop feeding, growing or reproducing or causing death of the target coleopteran and/or hemipteran plant pest. In some embodiments the plant pest is selected from the group consisting of Sitophilus oryzae (So; rice weevil), Diabrotica virgifera virgifera (Dv; western corn rootworm), Diabrotica undecimpunctata howardi (Du; southern corn rootworm), Diabrotica barberi (Db; northern corn rootworm), Phyllotreta armoraciae (Pa; horseradish flea beetle), Phyllotreta nemorum (Pn; turnip flea beetle), Phyllotreta cruciferae (Pu; crucifer flea beetle), Phyllotreta striolata (Ps; striped flea beetle), Phyllotreta atra (Pt; flea beetle), Psylliodes chrysocephala (Pc; cabbage-stem flea beetle), Meligethes aeneus (Ma; pollen beetle), Ceutorhynchus assimilis (Ca; cabbage seedpod weevil), Leptinotarsa decemlineata (Ld; Colorado potato beetle), and/or the hemipteran insect pest is selected from the group consisting of Nezara viridula (Nv; green stink bug), Euschistus heros (Eh; brown stink bug), and Piezodorus guildinii (Pg; red-banded stink bug). In another embodiment, the transgenic plant and transgenic seed is a corn plant or com seed. In another embodiment, the transgenic corn plant is provided by crossing a first transgenic corn plant comprising a dsRNA with a transgenic com plant comprising a transgenic event selected from the group consisting of MIR604, Event 5307, DAS51922-7, MON863 and MON88017.

In other embodiments, the transgenic plant is a transgenic corn plant and the target pest is a Diabrotica insect pest. In still other embodiments, the Diabrotica insect pest is selected from the group consisting of Diabrotica virgifera virgifera (Dv; western corn rootworm), Diabrotica undecimpunctata howardi (Du; southern com rootworm), and Diabrotica barberi (Db; northern corn rootworm).

In other embodiments, the transgenic organism is selected from, but not limited to, the group consisting of: yeast, fungi, algae, bacteria, virus or an arthropod expressing the molecules of the invention. In some embodiments, the transgenic organism is a virus, for example an insect baculovirus that expresses the molecules of the invention upon infection of an insect host. Such a baculovirus is likely more virulent against the target insect than the wildtype untransformed baculovirus. In other embodiments the transgenic organism is a transgenic bacterium that is applied to an environment where a target pest occurs or is known to have occurred. In some embodiments, non-pathogenic symbiotic bacteria, which are able to live and replicate within plant tissues, so-called endophytes, or non-pathogenic symbiotic bacteria, which are capable of colonizing the phyllosphere or the rhizosphere, so-called epiphytes, are used. Such bacteria include bacteria of the genera Agrobacterium, Alcaligenes, Azospirillum, Azotobacter, Bacillus, Clavibacter, Enterobacter, Erwinia, Flavobacter, Klebsiella, Pseudomonas, Rhizobium, Serratia, Streptomyces and Xanthomonas. Symbiotic fungi, such as Trichoderma and Gliocladium are also possible hosts for expression of the molecules of the invention, for the same purpose.

In some embodiments, the invention encompasses transgenic plants, or parts thereof, comprising an interfering RNA molecule and/or a nucleic acid molecule encoding a dsRNA and/or Hsc70-4 protein, a chimeric nucleic acid molecule, and/or a composition of the invention, wherein the transgenic plant has enhanced resistance to a coleopteran insect, hemipteran insect, or Diabrotica insect as compared to a control. In other embodiments, the transgenic plant, or part thereof, is a transgenic corn plant, or part thereof. The invention further encompasses transgenic seed of the transgenic plants of the invention, wherein the transgenic seed comprises an interfering RNA molecule, a nucleic acid construct, a chimeric nucleic acid molecule, an artificial plant microRNA precursor molecule and/or a composition of the invention. In some embodiments the transgenic seed is a transgenic com seed. Plants transformed in accordance with the present invention may be monocots or dicots and include, but are not limited to, corn, wheat, barley, rye, sweet potato, bean, pea, chicory, lettuce, cabbage, cauliflower, broccoli, turnip, radish, spinach, asparagus, onion, garlic, pepper, celery, squash, pumpkin, hemp, zucchini, apple, pear, quince, melon, plum, cherry, peach, nectarine, apricot, strawberry, grape, raspberry, blackberry, pineapple, avocado, papaya, mango, banana, soybean, tomato, sorghum, sugarcane, sugar beet, sunflower, rapeseed, clover, tobacco, carrot, cotton, alfalfa, rice, potato, eggplant, cucumber, Arabidopsis, and woody plants such as coniferous and deciduous trees. In further embodiments, the transgenic plant is a transgenic corn plant.

Expression of the Hsc70-4 protein and/or interfering RNA molecule in transgenic plants may be driven by regulatory sequences comprising promoters that function in plants. The choice of promoter will vary depending on the temporal and spatial requirements for expression, and also depending on the (insect) pest target species. Thus, expression of the interfering RNAs of this invention in leaves, in stalks or stems, in ears, in inflorescences (e.g. spikes, panicles, cobs, etc.), in roots, and/or seedlings is contemplated. In many cases, however, protection against more than one type of (insect) pest is sought, and thus expression in multiple tissues is desirable. Although many promoters from dicotyledons have been shown to be operational in monocotyledons and vice versa, ideally dicotyledonous promoters are selected for expression in dicotyledons, and monocotyledonous promoters for expression in monocotyledons. However, there is no restriction to the provenance of selected promoters; it is sufficient that they are operational in driving the expression of the dsRNA or siRNA in the desired cell.

Promoters useful with the invention include, but are not limited to, those that drive expression of a nucleotide sequence constitutively, those that drive expression when induced, and those that drive expression in a tissue- or developmentally-specific manner. These various types of promoters are known in the art. In some embodiments, tissue-specific/tissue-preferred promoters can be used. Tissue-specific or tissue-preferred expression patterns include, but are not limited to, green tissue specific or preferred, root specific or preferred, stem specific or preferred, and flower specific or preferred. In addition, promoters functional in plastids can be used. In some embodiments of the invention, inducible promoters can be used. In further aspects, the nucleotide sequences of the invention can be operably associated with a promoter that is wound inducible or inducible by pest or pathogen infection (e.g., an insect or nematode plant pest). In some embodiments of the present invention, a “minimal promoter” or “basal promoter” is used. A minimal promoter is capable of recruiting and binding RNA polymerase II complex and its accessory proteins to permit transcriptional initiation and elongation. In some embodiments, a minimal promoter is constructed to comprise only the nucleotides/nucleotide sequences from a selected promoter that are required for binding of the transcription factors and transcription of a nucleotide sequence of interest that is operably associated with the minimal promoter including but not limited to TATA box sequences. In other embodiments, the minimal promoter lacks cis sequences that recruit and bind transcription factors that modulate (e.g., enhance, repress, confer tissue specificity, confer inducibility or repressibility) transcription. A minimal promoter is generally placed upstream (i.e., 5′) of a nucleotide sequence to be expressed. Thus, nucleotides/nucleotide sequences from any promoter useable with the present invention can be selected for use as a minimal promoter.

In some embodiments, a recombinant nucleic acid molecule of the invention can be an “expression cassette.” As used herein, “expression cassette” means a recombinant nucleic acid molecule comprising a nucleotide sequence of interest (e.g., the nucleotide sequences of the invention), wherein the nucleotide sequence is operably associated with at least a control sequence (e.g., a promoter) encoding the Hsc70-4 protein and/or interfering RNA molecule. Thus, some embodiments of the invention provide expression cassettes designed to express nucleotides sequences encoding the Hsc70-4 protein of the invention. In this manner, for example, one or more plant promoters operably associated with one or more nucleotide sequences of the invention are provided in expression cassettes for expression in a com plant, plant part and/or plant cell.

An expression cassette of the invention also can include a nucleotide sequence for a selectable marker, which can be used to select a transformed plant, plant part and/or plant cell. As used herein, “selectable marker” means a nucleotide sequence that when expressed imparts a distinct phenotype to the plant, plant part and/or plant cell expressing the marker and thus allows such transformed plants, plant parts and/or plant cells to be distinguished from those that do not have the marker. Such a nucleotide sequence may encode either a selectable or screenable marker, depending on whether the marker confers a trait that can be selected for by chemical means, such as by using a selective agent (e.g., an antibiotic, herbicide, or the like), or on whether the marker is simply a trait that one can identify through observation or testing, such as by screening (e.g., the R-locus trait). Of course, many examples of suitable selectable markers are known in the art and can be used in the expression cassettes described herein. Examples of selectable markers include, but are not limited to, a nucleotide sequence encoding neo or nptl I, which confers resistance to kanamycin, G418, and the like (Potrykus et al. (1985) Mol. Gen. Genet. 199:183-188); a nucleotide sequence encoding bar, which confers resistance to phosphinothricin; a nucleotide sequence encoding an altered 5-enolpyruvylshikimate-3-phosphate (EPSP) synthase, which confers resistance to glyphosate (Hinchee et al. (1988) Biotech. 6:915-922); a nucleotide sequence encoding a nitrilase such as bxn from Klebsiella ozaenae that confers resistance to bromoxynil (Stalker et al. (1988) Science 242:419-423); a nucleotide sequence encoding an altered acetolactate synthase (ALS) that confers resistance to imidazolinone, sulfonylurea or other ALS-inhibiting chemicals (EP Patent Application No. 154204); a nucleotide sequence encoding a methotrexate-resistant dihydrofolate reductase (DHFR) (Thillet et al. (1988) J. Biol. Chem. 263:12500-12508); a nucleotide sequence encoding a dalapon dehalogenase that confers resistance to dalapon; a nucleotide sequence encoding a mannose-6-phosphate isomerase (also referred to as phosphomannose isomerase (PMI)) that confers an ability to metabolize mannose (U.S. Pat. Nos. 5,767,378 and 5,994,629); a nucleotide sequence encoding an altered anthranilate synthase that confers resistance to 5-methyl tryptophan; and/or a nucleotide sequence encoding hph that confers resistance to hygromycin. One of skill in the art is capable of choosing a suitable selectable marker for use in an expression cassette of the invention.

An expression cassette of the invention also can include polynucleotides that encode other desired traits. Such desired traits can be other polynucleotides which confer insect resistance, or which confer nematode resistance, or other agriculturally desirable traits. Such polynucleotides can be stacked with any combination of nucleotide sequences to create plants, plant parts or plant cells having the desired phenotype. Stacked combinations can be created by any method including, but not limited to, cross breeding plants by any conventional methodology, or by genetic transformation. If stacked by genetically transforming the plants, nucleotide sequences encoding additional desired traits can be combined at any time and in any order. For example, a single transgene can comprise multiple expression cassettes, such that multiple expression cassettes are introduced into the genome of a transformed cell at a single genomic location. Alternatively, a transgenic plant comprising one or more desired traits can be used as the target to introduce further traits by subsequent transformation. The additional nucleotide sequences can be introduced simultaneously in a co-transformation protocol with a nucleotide sequence, nucleic acid molecule, nucleic acid construct, and/or other composition of the invention, provided by any combination of expression cassettes. For example, if two nucleotide sequences will be introduced, they can be incorporated in separate cassettes (trans) or can be incorporated on the same cassette (cis). Expression of the nucleotide sequences can be driven by the same promoter or by different promoters. As used herein, the term “stacked” includes having the multiple traits present in the same plant or organism of interest. In one non-limiting example, “stacked traits” comprise a molecular stack where the sequences are physically adjacent to each other. A trait, as used herein, refers to the phenotype derived from a particular sequence or groups of sequences. In one embodiment, the molecular stack comprises at least one additional polynucleotide that also confers tolerance to at least one sequence that confers tolerance to glyphosate by the same and/or different mechanism and/or at least one additional polynucleotide that confers tolerance to a second herbicide.

Various methods can be used to introduce a sequence of interest into a host cell, plant or plant part. “Introducing” is intended to mean presenting to the host cell, plant, plant cell or plant part the polynucleotide or polypeptide in such a manner that the sequence gains access to the interior of a cell of the plant or organism. The methods of the disclosure do not depend on a particular method for introducing a sequence into an organism or a plant or plant part, only that the polynucleotide or polypeptides gains access to the interior of at least one cell of the organism or the plant. Methods for introducing polynucleotide or polypeptides into various organisms, including plants, are known in the art including, but not limited to, stable transformation methods, transient transformation methods, and virus-mediated methods.

It is further recognized that nucleotide sequences can be stacked at a desired genomic location using a site-specific recombination system. See, e.g., International Patent Application Publication Nos. WO 99/25821; WO 99/25854; WO 99/25840; WO 99/25855 and WO 99/2585. Thus, an expression cassette can include a coding sequence for one or more polypeptides for agronomic traits that primarily are of benefit to a seed company, grower or grain processor. A polypeptide of interest can be any polypeptide encoded by a polynucleotide sequence of interest. Non-limiting examples of polypeptides of interest that are suitable for production in plants include those resulting in agronomically important traits such as herbicide resistance (also sometimes referred to as «herbicide tolerance”), virus resistance, bacterial pathogen resistance, insect resistance, nematode resistance, and/or fungal resistance. See, e.g., U.S. Pat. Nos. 5,569,823; 5,304,730; 5,495,071; 6,329,504; and 6,337,431. Vectors suitable for plant transformation are described elsewhere in this specification. For Agrobacterium-mediated transformation, binary vectors or vectors carrying at least one T-DNA border sequence are suitable, whereas for direct gene transfer any vector is suitable and linear DNA containing only the construct of interest may be preferred. In the case of direct gene transfer, transformation with a single DNA species or co-transformation can be used (Schocher et al. Biotechnology 4:1093-1096 (1986)). For both direct gene transfer and Agrobacterium-mediated transfer, transformation is usually (but not necessarily) undertaken with a selectable marker that may provide resistance to an antibiotic (kanamycin, hygromycin or methotrexate) or a herbicide (basta). Plant transformation vectors of the invention may also comprise other selectable marker genes, for example, phosphomannose isomerase (pmi), which provides for positive selection of the transgenic plants as disclosed in U.S. Pat. Nos. 5,767,378 and 5,994,629, herein incorporated by reference, orphosphinotricin acetyltransferase (pat), which provides tolerance to the herbicide phosphinotricin (glufosinate). The choice of selectable marker is not, however, critical to the invention.

In other embodiments, a nucleic acid sequence of the invention is directly transformed into the plastid genome. Plastid transformation technology is extensively described in U.S. Pat. Nos. 5,451,513, 5,545,817, and 5,545,818, in PCT application no. WO 95/16783, and in McBride et al. (1994) Proc. Nati. Acad. Sci. USA 91, 7301-7305. The basic technique for chloroplast transformation involves introducing regions of cloned plastid DNA flanking a selectable marker together with the gene of interest into a suitable target tissue, e.g., using biolistics or protoplast transformation (e.g., calcium chloride or PEG mediated transformation). The 1 to 1.5 kb flanking regions, termed targeting sequences, facilitate homologous recombination with the plastid genome and thus allow the replacement or modification of specific regions of the plastome. Initially, point mutations in the chloroplast 16S rRNA and rpsl2 genes conferring resistance to spectinomycin and/or streptomycin are utilized as selectable markers for transformation (Svab, Z., Hajdukiewicz, P., and Maliga, P. (1990) Proc. Nati. Acad. Sci. USA 87, 8526-8530; Staub, J. M., and Maliga, P. (1992) Plant Cell 4, 39-45). This resulted in stable homoplasmic transformants at a frequency of approximately one per 100 bombardments of target leaves. The presence of cloning sites between these markers allowed creation of a plastid targeting vector for introduction of foreign genes (Staub, J. M., and Maliga, P. (1993) EMBO J. 12, 601-606). Substantial increases in transformation frequency are obtained by replacement of the recessive rRNA or r-protein antibiotic resistance genes with a dominant selectable marker, the bacterial aadA gene encoding the spectinomycin-cletoxifying enzyme aminoglycoside-3′-adenyltransf erase (Svab, Z., and Maliga, P. (1993) Proc. Natl. Acad. Sci. USA 90, 913-917). Previously, this marker had been used successfully for high-frequency transformation of the plastid genome of the green alga Chlamydomonas reinhardtii (Goldschmidt-Clermont, M. (1991) Nucl. Acids Res. 19:4083-4089). Other selectable markers useful for plastid transformation are known in the art and encompassed within the scope of the invention. Typically, approximately 15-20 cell division cycles following transformation are required to reach a homoplastidic state. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% of the total soluble plant protein. In a preferred embodiment, a nucleic acid sequence of the present invention is inserted into a plastid-targeting vector and transformed into the plastid genome of a desired plant host. Plants homoplastic for plastid genomes containing a nucleic acid sequence of the present invention are obtained, and are preferentially capable of high expression of the nucleic acid sequence.

“Stable transformation” is intended to mean that the nucleotide construct introduced into a plant integrates into the genome of the plant or organism of interest and is capable of being inherited by the progeny thereof. “Transient transformation” is intended to mean that a polynucleotide is introduced into the plant or organism of interest and does not integrate into the genome of the plant or organism or a polypeptide is introduced into a plant or organism. Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al (1986) Proc. Natl. Acad. Sci. USA 83:5602-5606, Agrobacterium-mediated transformation (U.S. Pat. Nos. 5,563,055 and 5,981,840), direct gene transfer (Paszkowski et al. (1984) EMBO J. 3:2717-2722), and ballistic particle acceleration (see, for example, U.S. Pat. Nos. 4,945,050; 5,879,918; 5,886,244; and, 5,932,782; Tomes et al. (1995) in Plant Cell, Tissue, and Organ Culture: Fundamental Methods, ed. Gamborg and Phillips (Springer-Verlag, Berlin); McCabe et al. (1988) Biotechnology 6:923-926); and Led transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); U.S. Pat. Nos. 5,240,855; 5,322,783; and, 5,324,646; Klein et al. (1988) Plant Physiol. 91:440-444 (maize); Fromm et al. 1990) Biotechnology 8:833-839 (maize); Hooykaas-Van Slogteren et al. (1984) Nature (London) 311:763-764; U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the Hsc70-4 sequences or active variants or fragments thereof can be provided to a plant using a variety of transient transformation methods. Such transient transformation methods include, but are not limited to, the introduction of the Hsc70-4 protein or active variants and fragments thereof directly into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91:2176-2180 and Hush et al. (1994) The Journal of Cell Science 707:775-784, all of which are herein incorporated by reference.

In other embodiments, the Hsc70-4 polynucleotide disclosed herein or active variants and fragments thereof may be introduced into plants by contacting plants with a virus or viral nucleic acids. Generally, such methods involve incorporating a nucleotide construct of the disclosure within a DNA or RNA molecule. It is recognized that the Hsc70-4 sequence may be initially synthesized as part of a viral polyprotein, which later may be processed by proteolysis in vivo or in vitro to produce the desired recombinant protein. Further, it is recognized that promoters disclosed herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853, all of which are herein incorporated by reference. Briefly, the polynucleotide disclosed herein can be contained in transfer cassette flanked by two non-recombinogenic recombination sites. The transfer cassette is introduced into a plant having stably incorporated into its genome a target site which is flanked by two non-recombinogenic recombination sites that correspond to the sites of the transfer cassette. An appropriate recombinase is provided and the transfer cassette is integrated at the target site. The polynucleotide of interest is thereby integrated at a specific chromosomal position in the plant genome. Other methods to target polynucleotides are set forth in WO 2009/114321 (herein incorporated by reference), which describes “custom” meganucleases produced to modify plant genomes, in particular the genome of maize. See, also, Gao et al. (2010) Plant Journal 7:176-187

In one embodiment, the invention provides a method of modifying a plant to express dsRNA and/or Hsc70-4 by genomic modification. In an embodiment, the genomic modification is introduced by guide RNA associated Cas9, guide RNA associated Cpf1 or any other CRISPR-associated endonuclease systems, TALEN, zinc finger endonucleases, and site-specific meganucleases. The Cas endonuclease can be introduced into a cell (provided to a cell) by any method known in the art, for example, but not limited to transient introduction methods, transfection, microinjection, and/or topical application or indirectly via recombination constructs. Bortesi and Fischer [Bortesi and Fischer, Biotechnology Advances (2015) 33: 41-52] discuss the use of CRISPR-Cas9 technology in plants as compared to ZFNs and TALENs, and Basak and Nithin [Basak and Nithin, Front Plant Sci. (2015) 6: 1001] teach that CRISPR-Cas9 technology has been applied for knockdown of protein-coding genes in model plants such as Arabidopsis and tobacco and crops including wheat, maize, and rice. WO202001834416, published on Sep. 17, 2020 provides additional methods of plant genome gene editing, in addition to methods of producing dsRNA for pest control.

In an embodiment, a method of producing a modified plant with an alteration in insecticidal characteristics includes (a) introducing into a regenerable plant cell a guide RNA, Cas endonuclease such that the guide RNA targets an endogenous genomic locus that encodes a polypeptide having an amino acid sequence that is at least 90% identical to SEQ ID NOS: 1-10; (b) regenerating a modified plant from the regenerable plant cell after step (a), wherein the modified plant comprises in its genome one or more modifications at the endogenous genomic locus; and (c). selecting a modified plant of (b), wherein the modified plant exhibits an alteration in resistance to pests when compared to a control plant not comprising the one or more modifications. In some embodiments, gene editing may be facilitated through the induction of a double-stranded break (DSB) or single strand break (e.g., nicking) in a defined position in the genome near the desired alteration. DSBs can be induced using any DSB-inducing agent available, including, but not limited to, TALENS, meganucleases, zinc finger nucleases, Cas9-gRNA systems (based on bacterial CRISPR-Cas systems), guided cpfl endonuclease systems, and the like. In some embodiments, the introduction of a DSB can be combined with the introduction of a polynucleotide expressing Hsc70-4 and/or a desired dsRNA. The endonuclease can be provided to a cell by any method known in the art, for example, but not limited to transient introduction methods, transfection, microinjection, and/or topical application or indirectly via recombination constructs. The endonuclease can be provided as a protein or as a guided polynucleotide complex directly to a cell or indirectly via recombination constructs. The endonuclease can be introduced into a cell transiently or can be incorporated into the genome of the host cell using any method known in the art. In the case of a CRISPR-Cas system, uptake of the endonuclease and/or the guided polynucleotide into the cell can be facilitated with a Cell Penetrating Peptide (CPP) as described in WO2016073433 published May 12, 2016. TAL effector nucleases (TALEN) are a class of sequence-specific nucleases that can be used to make double-strand breaks at specific target sequences in the genome of a plant or other organism. (Miller et al. (2011) Nature Biotechnology 29:143-148). Endonucleases are enzymes that cleave the phosphodiester bond within a polynucleotide chain. Endonucleases include restriction endonucleases, which cleave DNA at specific sites without damaging the bases, and meganucleases, also known as homing endonucleases (HEases), which like restriction endonucleases, bind and cut at a specific recognition site, however the recognition sites for meganucleases are typically longer, about 18 bp or more (patent application PCT/US 12/30061, filed on Mar. 22, 2012). Meganucleases have been classified into four families based on conserved sequence motifs, the families are the LAGLIDADG (SEQ ID NO:108), GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. The naming convention for meganuclease is similar to the convention for other restriction endonuclease. Meganucleases are also characterized by prefix F-, I-, or PI- for enzymes encoded by free-standing ORFs, introns, and inteins, respectively. One step in the recombination process involves polynucleotide cleavage at or near the recognition site. The cleaving activity can be used to produce a double-strand break. For reviews of site-specific recombinases and their recognition sites, see, Sauer (1994) Curr Op Biotechnol 5:521-7; and Sadowski (1993) FASEB 7:760-7. In some examples the recombinase is from the Integrase or Resolvase families. Zinc finger nucleases (ZFNs) are engineered double-strand break inducing agents comprised of a zinc finger DNA binding domain and a double-strand-break-inducing agent domain. Recognition site specificity is conferred by the zinc finger domain, which typically comprising two, three, or four zinc fingers, for example having a C2H2 structure, however other zinc finger structures are known and have been engineered. Zinc finger domains are amenable for designing polypeptides which specifically bind a selected polynucleotide recognition sequence. ZFNs include an engineered DNA-binding zinc finger domain linked to a nonspecific endonuclease domain, for example nuclease domain from a Type Ils endonuclease such as Fokl. Additional functionalities can be fused to the zinc-finger binding domain, including transcriptional activator domains, transcription repressor domains, and methylases. In some examples, dimerization of nuclease domain is required for cleavage activity. Each zinc finger recognizes three consecutive base pairs in the target DNA. For example, a 3-finger domain recognized a sequence of 9 contiguous nucleotides, with a dimerization requirement of the nuclease, two sets of zinc finger triplets are used to bind an 18 nucleotides recognition sequence. The term “Cas gene” herein refers to a gene that is generally coupled, associated or close to, or in the vicinity of flanking CRISPR loci in bacterial systems. The terms “Cas gene”, “CRISPR-associated (Cas) gene” are used interchangeably herein. The term “Cas endonuclease” herein refers to a protein encoded by a Cas gene. A Cas endonuclease herein, when in complex with a suitable polynucleotide component, is capable of recognizing binding to, and optionally nicking or cleaving all or part of a specific DNA target sequence. A Cas endonuclease described herein comprises one or more nuclease domains. Cas endonucleases of the disclosure includes those having a HNH or HNH-like nuclease domain and/or a RuvC or RuvC-like nuclease domain. A Cas endonuclease of the disclosure includes a Cas9 protein, a Cpf1 protein, a C2cl protein, a C2c2 protein, a C2c3 protein, Cas3, Cas 5, Cas7, Cas8, Cas 10, or complexes of these. As used herein, the terms “guide polynucleotide/Cas endonuclease complex”, “guide polynucleotide/Cas endonuclease system”, “guide polynucleotide/Cas complex”, “guide polynucleotide/Cas system”, “guided Cas system” are used interchangeably herein and refer to at least one guide polynucleotide and at least one Cas endonuclease that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double strand break) the DNA target site. A guide polynucleotide/Cas endonuclease complex herein can comprise Cas protein(s) and suitable polynucleotide component(s) of any of the four known CRISPR systems (Horvath and Barrangou, 2010, Science 327:167-170) such as a type I, II, or III CRISPR system. A Cas endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas protein. Such recognition and cutting of a target sequence by a Cas endonuclease typically occurs if the correct protospacer-adjacent motif (PAM) is located at or adjacent to the 3′ end of the DNA target sequence. Alternatively, a Cas protein herein may lack DNA cleavage or nicking activity, but can still specifically bind to a DNA target sequence when complexed with a suitable RNA component. (See also U.S. Patent Application US 2015-0082478 A1, published on Mar. 19, 2015 and US 2015-0059010 A1, published on Feb. 26, 2015, both are hereby incorporated in its entirety by reference). A guide polynucleotide/Cas endonuclease complex can cleave one or both strands of a DNA target sequence. A guide polynucleotide/Cas endonuclease complex that can cleave both strands of a DNA target sequence typically comprise a Cas protein that has all of its endonuclease domains in a functional state (e.g., wild type endonuclease domains or variants thereof retaining some or all activity in each endonuclease domain). Non-limiting examples of Cas9 nickases suitable for use herein are disclosed in U.S. Patent Appl. Publ. No. 2014/0189896, which is incorporated herein by reference. Other Cas endonuclease systems have been described in PCT patent applications PCT/US 16/32073, filed May 12, 2016 and PCT/US 16/32028 filed May 12, 2016, both applications incorporated herein by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present disclosure provides transformed seed (also referred to as “transgenic seed”) having a polynucleotide disclosed herein, for example, as part of an expression cassette, stably incorporated into their genome. Transformed plant cells which are derived by plant transformation techniques, including those discussed above, can be cultured to regenerate a whole plant which possesses the transformed genotype (i.e., a Hsc70-4 polynucleotide), and thus the desired phenotype, such as acquired resistance (i.e., tolerance) to glyphosate or a glyphosate analog. For transformation and regeneration of maize see, Gordon-Kamm et al., The Plant Cell, 2:603-618 (1990). Plant regeneration from cultured protoplasts is described in Evans et al. (1983) Protoplasts Isolation and Culture, Handbook of Plant Cell Culture, pp 124-176, Macmillan Publishing Company, New York; and Binding (1985) Regeneration of Plants, Plant Protoplasts pp 21-73, CRC Press, Boca Raton. Regeneration can also be obtained from plant callus, explants, organs, or parts thereof. Such regeneration techniques are described generally in Klee et al. (1987) Ann Rev of Plant Phys 38:467.

In vegetatively propagated crops, mature transgenic plants can be propagated by the taking of cuttings or by tissue culture techniques to produce multiple identical plants. Selection of desirable transgenics is made and new varieties are obtained and propagated vegetatively for commercial use. In seed propagated crops, mature transgenic plants can be self-crossed to produce a homozygous inbred plant. The inbred plant produces seed containing the newly introduced heterologous nucleic acid. These seeds can be grown to produce plants that would produce the selected phenotype. Parts obtained from the regenerated plant, such as flowers, seeds, leaves, branches, fruit, and the like are included, provided that these parts comprise cells comprising the Hsc70-4 nucleic acid. Progeny and variants, and mutants of the regenerated plants are also included, provided that these parts comprise the introduced nucleic acid sequences. In one embodiment, a homozygous transgenic plant can be obtained by sexually mating (selfing) a heterozygous transgenic plant that contains a single added heterologous nucleic acid, germinating some of the seed produced and analyzing the resulting plants produced for altered cell division relative to a control plant (i.e., native, non-transgenic). Back-crossing to a parental plant and out-crossing with a non-transgenic plant are also contemplated.

Transgenic plants or seed of the invention can also be treated with an insecticide or insecticidal seed coating as described in U.S. Pat. Nos. 5,849,320 and 5,876,739, herein incorporated by reference. Where both the insecticide or insecticidal seed coating and the transgenic plant or seed of the invention are active against the same target insect, for example a coleopteran pest, a hemipteran pest or a Diabrotica target pest, the combination is useful (i) in a method for further enhancing activity of the composition of the invention against the target insect, and (ii) in a method for preventing development of resistance to the composition of the invention by providing yet another mechanism of action against the target insect. Thus, the invention provides a method of enhancing control of a coleopteran insect population, a hemipteran insect population, or a Diabrotica insect population comprising providing a transgenic plant or seed of the invention and applying to the plant or the seed an insecticide or insecticidal seed coating to a transgenic plant or seed of the invention. Examples of such insecticides and/or insecticidal seed coatings include, without limitation, a carbamate, a pyrethroid, an organophosphate, a friprole, a neonicotinoid, an organochloride, a nereistoxin, or a combination thereof. In another embodiment, the insecticide or insecticidal seed coating are selected from the group consisting of carbofuran, carbaryl, methomyl, bifenthrin, tefluthrin, permethrin, cyfluthrin, lambda-cyhalothrin cypermethrin, deltamethrin, chlorpyrifos, chlorethoxyfos, dimethoate, ethoprophos, malathion, methyl-parathion, phorate, terbufos, tebupirimiphos, fipronil, acetamiprid, imidacloprid, thiacloprid, thiamethoxam, endosulfan, bensultap, and a combination thereof. Commercial products containing such insecticides and insecticidal seed coatings include, without limitation, FURADAN® (carbofuran), LANATE® (methomyl, metomil, mesomile), SEVIN® (carbaryl), TALSTAR® (bifenthrin), FORCE® (tefluthrin), AMMO® (cypermethrin), CYMBUSH® (cypermethrin), DELTA GOLD® (deltamethrin), KARATE® (lambda-cyhalothrin), AMBUSH® (permethrin), POUNCE® (permethrin), BRIGADE® (bifenthrin), CAPTURE® (bifenthrin), PROSHIELD® (tefluthrin), WARRIOR® (lambda-cyhalothrin), DURSBAN® (chlorphyrifos) FORTRESS® (chlorethoxyfos), MOCAP® (ethoprop), THIMET® (phorate), AASTAR® (phorate, fluey thinate), RAMPART® (phorate), COUNTER® (terbufos), CYGON® (dimethoate), DICAPTHON, REGENT® (fipronil), CRUISER® (thiamethoxam), GAUCHO® (imidacloprid), PRESCRIBE® (imidacloprid), PONCHO® (clothianidin) and AZTEC® (cyfluthrin, tebupirimphos). The compositions of the invention can also be combined with other biological control agents to enhance control of a coleopteran insect, a hemipteran insect.

In further embodiments, the invention encompasses a biological sample from a transgenic plant, seed, or parts thereof, of the invention, wherein the sample comprises a nucleic acid that encodes the Hsc70-4 protein of the disclosure and optionally also at least one strand of a dsRNA as proposed above. In other embodiments, the invention encompasses a commodity product derived from a transgenic plant, seed, or parts thereof, of the invention. In some embodiments, the commodity product is selected from the group consisting of whole or processed seeds, beans, grains, kernels, hulls, meals, grits, flours, sugars, sugars, starches, protein concentrates, protein isolates, waxes, oils, extracts, juices, concentrates, liquids, syrups, feed, silage, fiber, paper or other food or product produced from plants. In other embodiments, the biological sample or commodity product is toxic to (insects) pests. In other embodiments, the transgenic plant is a transgenic corn plant.

### EXAMPLES

### Example 1: S2 Cells Internalize dsRNA when Grown in a Protein-Free Medium

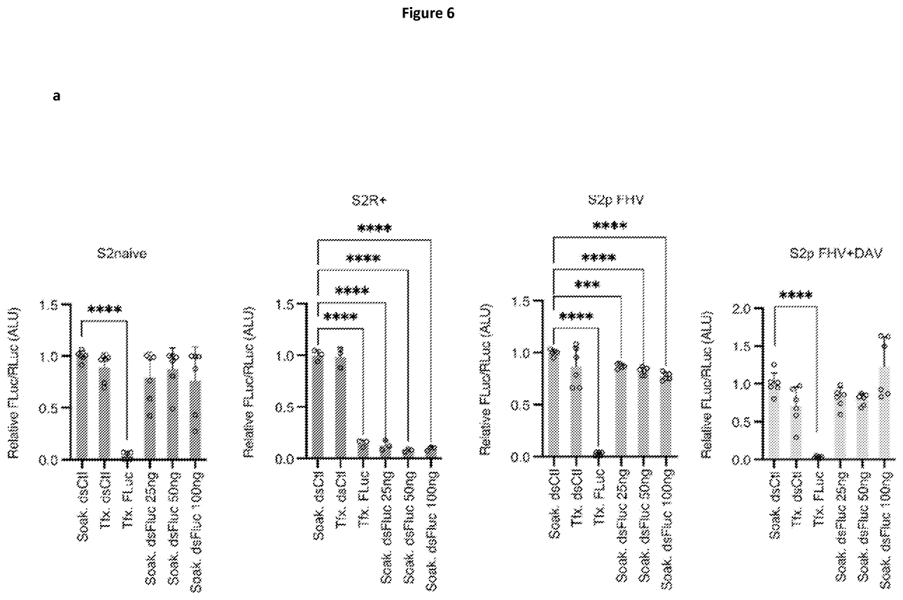

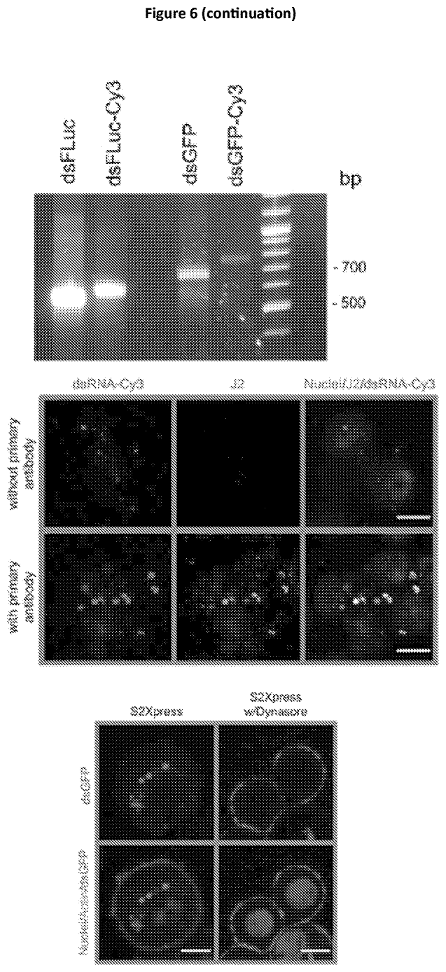

D. melanogaster S2 cells are widely used as a tool to study gene function because of their capacity to internalize dsRNA from the cell medium to induce gene silencing through RNAi. dsRNA internalization in S2 cells depends on the composition of the growth medium and on the specific cell line used. While S2 cells internalize dsRNA from the growth medium only in the absence of fetal bovine serum (FBS), the S2 receptor plus (S2R+)24 cell line can internalize dsRNA in the presence or absence of FBS25. With the objective of establishing an experimental model that would allow the inventors' to identify the proteins involved in the internalization of dsRNA from the extracellular milieu, the inventors first tested the dsRNA-internalization capability of the S2 and S2R+ cells from the inventors' stock. To do so, the inventors employed a commonly used Luciferase assay. Briefly, cells were initially transfected with plasmids expressing the Firefly and Renilla luciferases. Next, dsRNA molecules targeting the Firefly luciferase sequence (dsFluc) or an unrelated control gene sequence (dsCtl) were added to the cell growth medium. Finally, luciferase activity was quantified as an indirect measure of dsRNA internalization. In this assay, dsFluc is expected to reduce the expression of Firefly luciferase through RNAi in a dose-dependent manner. Thus, cells which internalize higher levels of dsRNA will exhibit less Firefly luciferase activity. The level of Firefly luciferase is normalized based on the level of Renilla luciferase to control for differences in transfection efficiency between cells. Compared to dsCtl-treatedcontrols, the inventors observed a decrease in Firefly luciferase activity when dsFluc was added to the growth medium of S2R+ cells (FIG. 6a). This decrease was not observed for S2 cells. In agreement with previous reports25, these results indicate that S2R+ cells, but not S2 cells, are able to internalize dsRNA in the presence of FBS.

Considering that S2R+ cells are persistently infected with DCV, Drosophila A virus (DAV), Drosophila X virus (DXV), and Flock House virus (FHV)²⁶, the inventors wondered if the infection status of these cells could account for this difference in dsRNA internalization capacity. To test if S2 cells infected with viruses can internalize dsRNA, the inventors repeated the Luciferase assay using two different S2 cell lines previously established in the inventors' laboratory that are persistently infected with either FHV (S2p FHV) or FHV and DAV (S2p FHV+DAV)²⁷ (FIG. 6a). The inventors observed a small, but significant decrease in Firefly luciferase activity in S2p FHV cells treated with dsFluc compared to those treated with dsCtl. However, no differences in Firefly luciferase activity were observed between dsFluc- and dsCtl-treated S2p FHV+DAV cells. These results suggest that, even if the infection status of the cells might have an impact on dsRNA internalization capacity, viral infection is not the primary determinant of their dsRNA internalization ability.

To avoid the use of an infected cell line, the inventors attempted to develop a model for dsRNA internalization in non-infected cells. The inventors routinely culture S2 cells in Schneider cell culture medium supplemented with 10% FBS. Considering that FBS prevents dsRNA internalization by S2 cells, the inventors adapted the inventors' uninfected S2 cell line (hereafter referred to as S2naive) to growth in Insect-XPRESS™ protein-free medium, which does not contain FBS or any other proteins. The inventors then tested dsRNA internalization in the newly adapted cells (hereafter referred to as S2Xpress). First, the inventors used the Luciferase assay to compare the dsRNA internalization abilities of S2naive cells (grown in FBS-containing media) and S2Xpress cells (grown without FBS). The inventors found a significant decrease in Firefly luciferase activity for S2Xpress cells treated with dsFluc compared to dsCtl. As the inventors previously observed, dsFluc treatment did not significantly alter Firefly luciferase activity in S2naive cells grown in the presence of FBS that cannot internalize sRNA (FIG. 1a). Furthermore, dsRNA internalization was dose-dependent in S2Xpress cells (FIG. 1b).

| | p-value (vs. Soak. | p-value (vs. Soak. |
|---|---|---|
| Treatment | dsCtl) S2naive | dsCtl) S2Xpress |
| Tfx. dsCtl | ns | ns |
| w/o dsRNA | ns | ns |
| Tfx. dsFLuc | 0.0006 | 0.0002 |
| Soak. dsFLuc 100 ng | ns | 0.0003 |
| Soak. dsFLuc 75 ng | ns | <0.0001 |
| Soak. dsFLuc 50 ng | ns | 0.0003 |
| Soak. dsFLuc 25 ng | ns | 0.0001 |
| Soak. dsFLuc 12.5 ng | ns | <0.0001 |
| Soak. dsFLuc 6.25 ng | ns | <0.0001 |
| Soak. dsFLuc 3.13 ng | ns | 0.0117 |
| Soak. dsFLuc 1.56 ng | ns | 0.0493 |
| Soak. dsFLuc 0.78 ng | ns | 0.0181 |

Table displaying the p-values of panel (b) from FIG. 1.
ALU: arbitrary light units;
Soak.: soaking;
Tfx.: transfection

The inventors also found that the substituting Insect-XPRESS™ protein-free medium for 10% FBS-Schneider medium during incubation with dsRNA in S2Xpress cells had little effect on the internalization, given that significant silencing was still observed in these conditions (FIG. 1c).

As a more direct readout of dsRNA internalization, the inventors next visualized internalization of dsRNA labeled with Cy3 (dsRNA-Cy3) using fluorescence confocal imaging. As before, the inventors found that S2Xpress cells, but not S2naive cells, internalized dsRNA-Cy3 (FIG. 1d). Receptor-mediated endocytosis was previously suggested to be involved in dsRNA internalization in S2 cells^{18,19}. To determine if this was also the case for S2Xpress cells, the inventors tested the effect of Dynasore, an inhibitor of dynamin-dependent endocytosis, on internalization of dsRNA using the inventors' dsRNA-Cy3 internalization assay. To this end, the inventors first treated the cells with Dynasore prior to addition of dsRNA-Cy3 to the culture medium. While dsRNA-Cy3 was detected as a punctuated pattern within the cytoplasm of S2Xpress cells, dsRNA-Cy3 was localized to the cell surface when cells were treated with Dynasore (FIG. 1d). Furthermore, dsRNA internalization by S2Xpress cells was not sequence-dependent, since dsRNA internalization was still evident when the inventors used a different dsRNA (FIG. 6d). Finally, the inventors found that internalization of nucleic acids by S2Xpress cells was restricted to dsRNA, since when the inventors performed the internalization assay using dsDNA-Cy3 or siRNA-Cy3, the inventors only saw a few spots or no spots inside the cells, respectively (FIG. 1e). Together these results show that S2Xpress cells can specifically internalize dsRNA from their environment to trigger the siRNA pathway. Since S2Xpress cells were obtained after adaptation of S2naive cells to a protein-free medium, and because only S2Xpress can internalize dsRNA, comparison of S2naive cells with S2Xpress cells provided the inventors' with an appropriate model system to search for proteins involved in dsRNA binding and internalization in D. melanogaster.

### Example 2: Complementary Proteomic Approaches Identify Cell Surface Proteins with dsRNA Binding Abilities

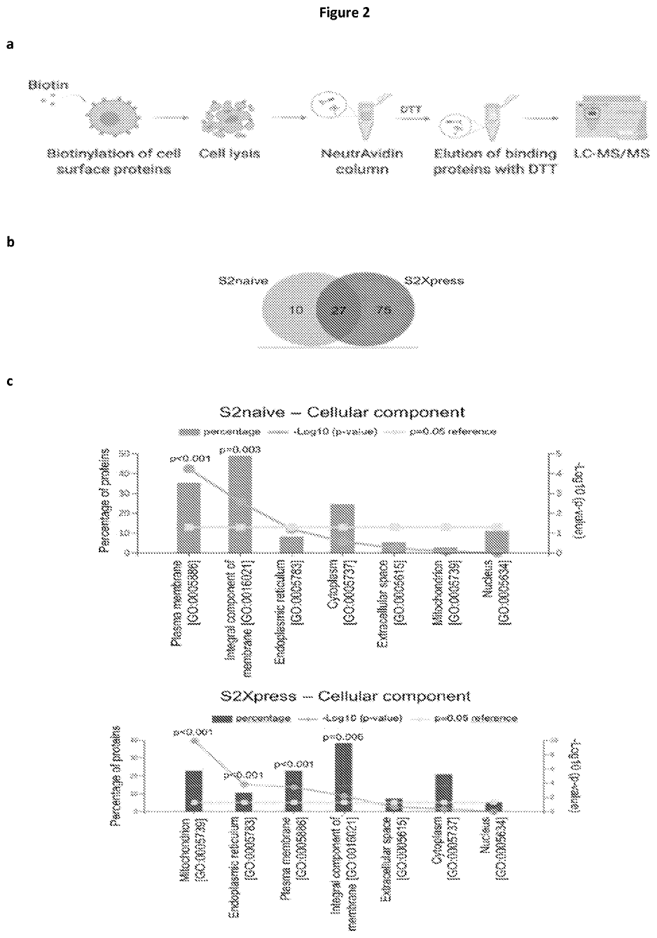

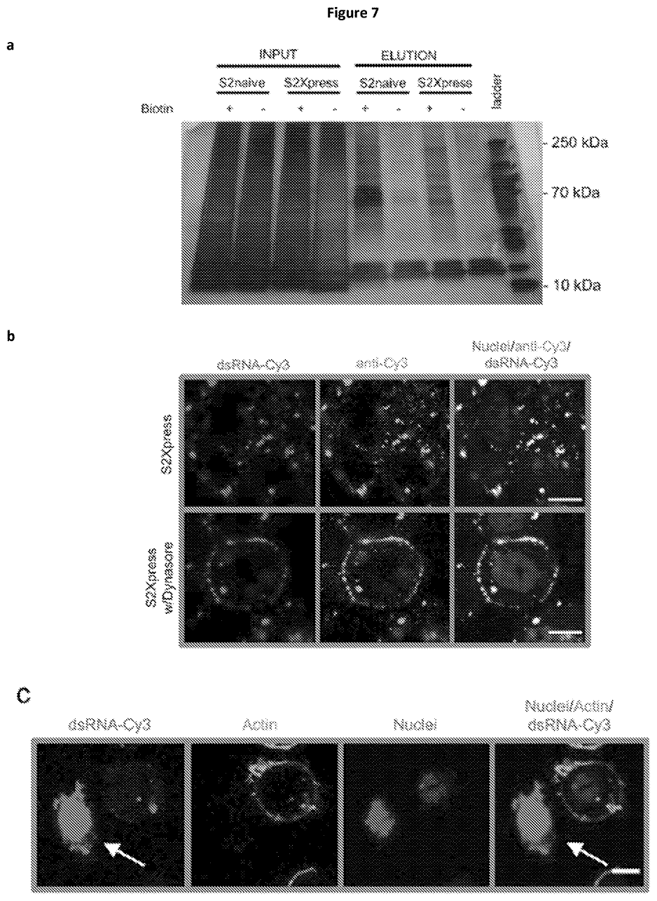

Given that S2Xpress cells, but not S2naive cells, can internalize dsRNA, the inventors hypothesized that differences in the composition of the plasma membranes of these two cell types might underlie differences in dsRNA internalization ability. One possibility is that the dsRNA receptor(s) is present at the surface of S2Xpress cells, but not S2naive cells. To evaluate differences in cell surface components, the inventors purified the cell surface proteins from both cell lines and determined their identity by liquid chromatography-mass spectrometry (LC-MS/MS) (FIG. 2a). Clear differences in the protein compositions of the plasma membranes between the two cell lines were observed by SDS-PAGE of the purified membrane proteins followed by silver staining (FIG. 7a). After analyzing the cell surface protein purifications to determine protein identity, the inventors were able to identify 37 proteins in extracts from S2naive cells and 102 proteins in extracts from S2Xpress cells, respectively (FIG. 2b, Table 1). Only 27 of these proteins were present in both cell types. A cellular component analysis showed a high percentage of known membrane proteins were detected for both cell types, validating the purification protocol (FIG. 2c). For S2xpress cells, a high percentage of mitochondrion proteins was also detected.

TABLE 1
List of proteins found in cell surface proteins purification (FIG. 2a-c)

| | | Gene | | | |
|---|---|---|---|---|---|
| Accession | Gene ID | Name | Description | S2Xpress | S2naive |
| Q7KTI8 | CG8222 | Pvr | PDGF-and VEGF-receptor related, | High | High |
| | | | isoform E OS = Drosophila | | |
| | | | melanogaster GN = Pvr PE = 4 SV = 1 | | |
| P13607 | CG5670 | Atpα | Sodium/potassium-transporting | High | High |
| | | | ATPase subunit alpha | | |
| | | | OS = Drosophila melanogaster | | |
| | | | GN = Atpalpha PE = 1 SV = 3 | | |
| Q9VHX9 | CG2791 | CD98hc | CD98 heavy chain OS = Drosophila | High | High |
| | | | melanogaster GN = CD98hc PE = 2 | | |
| | | | SV = 1 | | |
| P54399 | CG6988 | Pdi | Protein disulfide-isomerase | High | High |
| | | | OS = Drosophila melanogaster | | |
| | | | GN = Pdi PE = 2 SV = 1 | | |
| P29844 | CG4147 | Hsc70-3 | Heat shock 70 kDa protein cognate | High | High |
| | | | 3 OS = Drosophila melanogaster | | |
| | | | GN = Hsc70-3 PE = 2 SV = 2 | | |
| A8Y516 | CG42595 | uex | Unextended, isoform E | High | High |
| | | | OS = Drosophila melanogaster | | |
| | | | GN = uex PE = 4 SV = 2 | | |
| Q95R48 | CG13610 | Orct2 | Organic cation transporter-like | High | High |
| | | | protein OS = Drosophila | | |
| | | | melanogaster GN = Orct2 PE = 2 | | |
| | | | SV = 2 | | |
| A8JTM7 | CG42611 | mgl | Megalin, isoform A OS = Drosophila | High | Not |
| | | | melanogaster GN = mgl PE = 4 SV = 2 | | Found |
| P11147 | CG4264 | Hsc70-4 | Heat shock 70 kDa protein cognate | High | Not |
| | | | 4 OS = Drosophila melanogaster | | Found |
| | | | GN = Hsc70-4 PE = 1 SV = 3 | | |
| Q3YMU0 | CG8983 | ERp60 | ERp60 (Fragment) OS = Drosophila | High | High |
| | | | melanogaster GN = ERp60 PE = 2 | | |
| | | | SV = 1 | | |
| P06605 | CG2512 | αTub84D | Tubulin alpha-3 chain | High | High |
| | | | OS = Drosophila melanogaster | | |
| | | | GN = alphaTub84D PE = 2 SV = 1 | | |
| Q9VHV3 | CG7800 | | CG7800 OS = Drosophila | High | High |
| | | | melanogaster GN = CG7800-RA | | |
| | | | PE = 2 SV = 2 | | |
| P35381 | CG3612 | blw | ATP synthase subunit alpha, | High | High |
| | | | mitochondrial OS = Drosophila | | |
| | | | melanogaster GN = blw PE = 1 SV = 2 | | |
| Q24048-2 | CG9261 | nrv2 | Isoform 2.1 of Sodium/potassium- | High | High |
| | | | transporting ATPase subunit beta-2 | | |
| | | | OS = Drosophila melanogaster | | |
| | | | GN = nrv2 | | |
| Q05825 | CG11154 | ATPsynβ | ATP synthase subunit beta, | High | Not |
| | | | mitochondrial OS = Drosophila | | Found |
| | | | melanogaster GN = ATPsyn-beta | | |
| | | | PE = 2 SV = 3 | | |
| M9NEZ5 | CG1634 | Nrg | Neuroglian, isoform G | High | Not |
| | | | OS = Drosophila melanogaster | | Found |
| | | | GN = Nrg PE = 4 SV = 1 | | |
| Q9VV75 | CG4169 | UQCR- | AT02348p OS = Drosophila | High | Not |
| | | C2 | melanogaster GN = CG4169 PE = 2 | | Found |
| | | | SV = 1 | | |
| P91679 | CG44402 | yin | Peptide transporter family 1 | High | High |
| | | | OS = Drosophila melanogaster | | |
| | | | GN = yin PE = 1 SV = 2 | | |
| Q94920 | CG6647 | porin | Voltage-dependent anion-selective | High | Not |
| | | | channel OS = Drosophila | | Found |
| | | | melanogaster GN = porin PE = 1 SV = 3 | | |
| Q7K0L5 | CG4827 | veil | LP01562p OS = Drosophila | High | Not |
| | | | melanogaster GN = veil PE = 2 SV = 1 | | Found |
| Q8IPG9 | CG31605 | Bsg | Basigin, isoform A OS = Drosophila | High | High |
| | | | melanogaster GN = Bsg PE = 2 SV = 1 | | |
| P83097 | CG31127 | Wsck | Putative tyrosine-protein kinase | High | High |
| | | | Wsck OS = Drosophila melanogaster | | |
| | | | GN = Wsck PE = 2 SV = 2 | | |
| Q8SZY4 | CG30104 | NT5E-2 | CG30104, isoform A OS = Drosophila | High | Not |
| | | | melanogaster GN = NT5E-2 PE = 2 | | Found |
| | | | SV = 1 | | |
| Q9VIE8 | CG9244 | mAcon1 | Aconitase, isoform B OS = Drosophila | High | Not |
| | | | melanogaster GN = Acon PE = 2 SV = 2 | | Found |
| O02649 | CG12101 | Hsp60A | 60 kDa heat shock protein, | High | Not |
| | | | mitochondrial OS = Drosophila | | Found |
| | | | melanogaster GN = Hsp60 PE = 1 | | |
| | | | SV = 3 | | |
| Q9VU58 | CG11051 | Nplp2 | Neuropeptide-like 2 OS = Drosophila | High | Not |
| | | | melanogaster GN = Nplp2 PE = 1 | | Found |
| | | | SV = 1 | | |
| Q24046 | CG9258 | nrv1 | Sodium/potassium-transporting | High | High |
| | | | ATPase subunit beta-1 | | |
| | | | OS = Drosophila melanogaster | | |
| | | | GN = nrv1 PE = 1 SV = 2 | | |
| P18489 | CG12210 | Syb | Synaptobrevin OS = Drosophila | High | High |
| | | | melanogaster GN = Syb PE = 2 SV = 3 | | |
| Q8MLQ5 | CG5594 | kcc | CG5594, isoform D OS = Drosophila | High | High |
| | | | melanogaster GN = kcc PE = 2 SV = 1 | | |
| Q9VCA2 | CG6331 | Orct | Organic cation transporter protein | Not | High |
| | | | OS = Drosophila melanogaster | Found | |
| | | | GN = Orct PE = 1 SV = 1 | | |
| Q9VKC2 | CG12317 | Jhl-21 | Amino acid transporter protein JHl- | High | High |
| | | | 21 OS = Drosophila melanogaster | | |
| | | | GN = Jhl-21 PE = 2 SV = 1 | | |
| Q4U2G8 | CG11961 | | CG11961, isoform A OS = Drosophila | High | Not |
| | | | melanogaster GN = CG11961 PE = 2 | | Found |
| | | | SV = 1 | | |
| Q8IQB7 | CG32032 | | CG32032 OS = Drosophila | High | High |
| | | | melanogaster GN = CG32032-RA | | |
| | | | PE = 2 SV = 1 | | |
| Q7K1L4 | CG8468 | | CG8468, isoform A OS = Drosophila | High | High |
| | | | melanogaster GN = CG8468 PE = 2 | | |
| | | | SV = 1 | | |
| Q9V438 | CG5809 | CaBP1 | Calcium-binding protein 1 | High | Not |
| | | | OS = Drosophila melanogaster | | Found |
| | | | GN = CaBP1 PE = 2 SV = 1 | | |
| Q9VAY2 | CG5520 | Gp93 | Glycoprotein 93 OS = Drosophila | High | Not |
| | | | melanogaster GN = Gp93 PE = 2 | | Found |
| | | | SV = 1 | | |
| Q9W266 | CG3413 | wdp | FI21560p1 OS = Drosophila | Not | High |
| | | | melanogaster GN = wdp PE = 2 SV = 1 | Found | |
| P29413 | CG9429 | Calr | Calreticulin OS = Drosophila | High | Not |
| | | | melanogaster GN = Crc PE = 1 SV = 2 | | Found |
| O62619 | CG7070 | Pyk | Pyruvate kinase OS = Drosophila | High | High |
| | | | melanogaster GN = PyK PE = 2 SV = 2 | | |
| Q9VFF0 | CG3731 | UQCR- | CG3731, isoform A OS = Drosophila | High | Not |
| | | C1 | melanogaster GN = CG3731 PE = 2 | | Found |
| | | | SV = 2 | | |
| Q9W436 | CG5905 | Nep1 | GH03315p OS = Drosophila | High | Not |
| | | | melanogaster GN = Nep1 PE = 2 | | Found |
| | | | SV = 2 | | |
| P84029 | CG17903 | Cyt-c-p | Cytochrome c-2 OS = Drosophila | High | Not |
| | | | melanogaster GN = Cyt-c-p PE = 1 | | Found |
| | | | SV = 2 | | |
| P17336 | CG6871 | Cat | Catalase OS = Drosophila | High | Not |
| | | | melanogaster GN = Cat PE = 1 SV = 2 | | Found |
| P13060 | CG2238 | eEF2 | Elongation factor 2 OS = Drosophila | High | Not |
| | | | melanogaster GN = EF2 PE = 1 SV = 4 | | Found |
| Q0E9L6 | CG17800 | Dscam1 | Down syndrome cell adhesion | Not | High |
| | | | molecule, isoform AV | Found | |
| | | | OS = Drosophila melanogaster | | |
| | | | GN = Dscam1 PE = 4 SV = 1 | | |
| P29845 | CG8542 | Hsc70-5 | Heat shock 70 kDa protein cognate | High | Not |
| | | | 5 OS = Drosophila melanogaster | | Found |
| | | | GN = Hsc70-5 PE = 1 SV = 2 | | |
| Q9VHJ8 | CG11963 | ScsβA | SkpA associated protein, isoform A | High | Not |
| | | | OS = Drosophila melanogaster | | Found |
| | | | GN = skap PE = 3 SV = 2 | | |
| P22700 | CG3725 | SERCA | Calcium-transporting ATPase | High | Not |
| | | | sarcoplasmic/endoplasmic reticulum | | Found |
| | | | type OS = Drosophila melanogaster | | |
| | | | GN = Ca-P60A PE = 1 SV = 2 | | |
| Q9VMB6 | CG31911 | Ent2 | Equilibrative nucleoside transporter | High | High |
| | | | 2 OS = Drosophila melanogaster | | |
| | | | GN = Ent2 PE = 2 SV = 1 | | |
| Q24372 | CG12369 | Lac | Lachesin OS = Drosophila | High | High |
| | | | melanogaster GN = Lac PE = 1 SV = 2 | | |
| Q86BM5 | CG13388 | Akap200 | A kinase anchor protein 200, | High | Not |
| | | | isoform D OS = Drosophila | | Found |
| | | | melanogaster GN = Akap200 PE = 4 | | |
| | | | SV = 1 | | |
| A1ZBL0 | CG9277 | βTub56D | Beta-Tubulin at 56D, isoform A | High | High |
| | | | OS = Drosophila melanogaster | | |
| | | | GN = betaTub56D PE = 2 SV = 1 | | |
| A1ZB83 | CG5174 | | CG5174, isoform B OS = Drosophila | High | Not |
| | | | melanogaster GN = CG5174 PE = 4 | | Found |
| | | | SV = 1 | | |
| Q9W2V9 | CG2221 | I(1)G0289 | LD40707p OS = Drosophila | High | High |
| | | | melanogaster GN = I(1)G0289 PE = 2 | | |
| | | | SV = 1 | | |
| Q94522 | CG1065 | Scsα1 | Succinyl-CoA ligase [ADP/GDP- | High | Not |
| | | | forming] subunit alpha, | | Found |
| | | | mitochondrial OS = Drosophila | | |
| | | | melanogaster GN = Scsalpha PE = 2 | | |
| | | | SV = 3 | | |
| Q9VF54 | CG44014 | | CG14872, isoform B OS = Drosophila | High | Not |
| | | | melanogaster GN = CG14872 PE = 4 | | Found |
| | | | SV = 1 | | |
| Q9VIW9 | CG16771 | Alp12 | CG16771, isoform A OS = Drosophila | High | Not |
| | | | melanogaster GN = CG16771 PE = 3 | | Found |
| | | | SV = 1 | | |
| Q95RU0 | CG12086 | cue | Protein cueball OS = Drosophila | Not | High |
| | | | melanogaster GN = cue PE = 1 SV = 1 | Found | |
| P07487 | CG8893 | Gapdh2 | Glyceraldehyde-3-phosphate | High | Not |
| | | | dehydrogenase 2 OS = Drosophila | | Found |
| | | | melanogaster GN = Gapdh2 PE = 1 | | |
| | | | SV = 2 | | |
| O18332 | CG3320 | Rab1 | FI01544p OS = Drosophila | Not | High |
| | | | melanogaster GN = Rab1 PE = 2 | Found | |
| | | | SV = 1 | | |
| Q94523 | CG17246 | SdhA | Succinate dehydrogenase | High | Not |
| | | | [ubiquinone] flavoprotein subunit, | | Found |
| | | | mitochondrial OS = Drosophila | | |
| | | | melanogaster GN = SdhA PE = 2 | | |
| | | | SV = 3 | | |
| P39018 | CG4464 | RpS19a | 40S ribosomal protein S19a | High | Not |
| | | | OS = Drosophila melanogaster | | Found |
| | | | GN = RpS19a PE = 1 SV = 3 | | |
| Q9VLC5 | CG3752 | Aldh | Aldehyde dehydrogenase | High | Not |
| | | | OS = Drosophila melanogaster | | Found |
| | | | GN = Aldh PE = 3 SV = 1 | | |
| Q9VQL7 | CG3523 | FASN1 | CG3523, isoform A OS = Drosophila | High | Not |
| | | | melanogaster GN = CG3523 PE = 4 | | Found |
| | | | SV = 1 | | |
| Q9Y1A7 | CG3297 | mnd | LD25378p OS = Drosophila | High | High |
| | | | melanogaster GN = mnd PE = 2 SV = 1 | | |
| Q7YU05 | CG7010 | Pdha | LD13846p OS = Drosophila | High | Not |
| | | | melanogaster GN = I(1)G0334 PE = 2 | | Found |
| | | | SV = 1 | | |
| P02828 | CG1242 | Hsp83 | Heat shock protein 83 | High | Not |
| | | | OS = Drosophila melanogaster | | Found |
| | | | GN = Hsp83 PE = 1 SV = 1 | | |
| A1Z729 | CG2064 | Rdh1 | CG2064 OS = Drosophila | High | Not |
| | | | melanogaster GN = CG2064 PE = 3 | | Found |
| | | | SV = 1 | | |
| Q9VH26 | CG3940 | CAH7 | CG3940 OS = Drosophila | High | Not |
| | | | melanogaster GN = CG3940 PE = 4 | | Found |
| | | | SV = 2 | | |
| Q9W227 | CG2852 | | Peptidyl-prolyl cis-trans isomerase | High | Not |
| | | | OS = Drosophila melanogaster | | Found |
| | | | GN = CG2852 PE = 2 SV = 1 | | |
| Q9VGQ1 | CG5214 | | CG5214 OS = Drosophila | High | Not |
| | | | melanogaster GN = CG5214 PE = 2 | | Found |
| | | | SV = 1 | | |
| Q6QU65 | CG7649 | Meltrin | ADAM metalloprotease | High | Not |
| | | | OS = Drosophila melanogaster | | Found |
| | | | GN = Meltrin PE = 2 SV = 1 | | |
| Q7K2E1 | CG8839 | | CG8839, isoform A OS = Drosophila | High | Not |
| | | | melanogaster GN = CG8839 PE = 2 | | Found |
| | | | SV = 1 | | |
| Q9VK56 | CG6214 | MRP | Multidrug-Resistance like protein 1, | High | Not |
| | | | isoform A OS = Drosophila | | Found |
| | | | melanogaster GN = MRP PE = 4 SV = 4 | | |
| Q9VWH4 | CG12233 | Idh3a | Probable isocitrate dehydrogenase | High | Not |
| | | | [NAD] subunit alpha, mitochondrial | | Found |
| | | | OS = Drosophila melanogaster | | |
| | | | GN = I(1)G0156 PE = 2 SV = 1 | | |
| Q7KB18 | CG15102 | Jheh2 | FI02870p OS = Drosophila | High | Not |
| | | | melanogaster GN = Jheh2 PE = 2 | | Found |
| | | | SV = 1 | | |
| Q9VLN9 | CG14273 | | CG14273 OS = Drosophila | High | Not |
| | | | melanogaster GN = CG14273 PE = 4 | | Found |
| | | | SV = 3 | | |
| Q7K010 | CG12845 | Tsp42Ef | RE01312p OS = Drosophila | High | Not |
| | | | melanogaster GN = Tsp42Ef PE = 2 | | Found |
| | | | SV = 1 | | |
| Q9VB46 | CG3373 | Hmu | FI18644p1 OS = Drosophila | High | Not |
| | | | melanogaster GN = Hmu PE = 2 SV = 1 | | Found |
| Q7JR61 | CG11140 | Aldh-III | Aldehyde dehydrogenase | High | Not |
| | | | OS = Drosophila melanogaster | | Found |
| | | | GN = Aldh-III PE = 2 SV = 1 | | |
| O18640 | CG7111 | Rack1 | Guanine nucleotide-binding protein | High | Not |
| | | | subunit beta-like protein | | Found |
| | | | OS = Drosophila melanogaster | | |
| | | | GN = Rack1 PE = 1 SV = 2 | | |
| Q7KJ73 | CG10106 | Tsp42Ee | FI02814p OS = Drosophila | High | Not |
| | | | melanogaster GN = Tsp42Ee PE = 2 | | Found |
| | | | SV = 1 | | |
| O18404 | CG7113 | scu | 3-hydroxyacyl-CoA dehydrogenase | High | Not |
| | | | type-2 OS = Drosophila melanogaster | | Found |
| | | | GN = scu PE = 1 SV = 1 | | |
| Q9VSD6 | CG7935 | msk | D-Importin 7/RanBP7 | High | Not |
| | | | OS = Drosophila melanogaster | | Found |
| | | | GN = msk PE = 2 SV = 1 | | |
| P48605 | CG8977 | CCT3 | T-complex protein 1 subunit gamma | High | Not |
| | | | OS = Drosophila melanogaster | | Found |
| | | | GN = Cctgamma PE = 2 SV = 2 | | |
| E1JHB4 | CG44122 | Piezo | Piezo, isoform G OS = Drosophila | High | Not |
| | | | melanogaster GN = Piezo PE = 4 | | Found |
| | | | SV = 1 | | |
| Q9VBA3 | CG46339 | | AT30002p OS = Drosophila | High | Not |
| | | | melanogaster GN = sda PE = 2 SV = 2 | | Found |
| A8JV19 | CG4829 | | CG4829, isoform E OS = Drosophila | High | Not |
| | | | melanogaster GN = CG4829 PE = 4 | | Found |
| | | | SV = 1 | | |
| Q8STG9 | CG9539 | Sec61α | DSec61alpha OS = Drosophila | High | Not |
| | | | melanogaster GN = Sec61alpha | | Found |
| | | | PE = 2 SV = 1 | | |
| Q9W260 | CG10382 | wrapper | GH03113p OS = Drosophila | High | Not |
| | | | melanogaster GN = wrapper PE = 2 | | Found |
| | | | SV = 2 | | |
| P29742 | CG9012 | Chc | Clathrin heavy chain OS = Drosophila | High | Not |
| | | | melanogaster GN = Chc PE = 1 SV = 1 | | Found |
| Q9VVT2 | CG3979 | Indy | Protein I'm not dead yet | Not | High |
| | | | OS = Drosophila melanogaster | Found | |
| | | | GN = Indy PE = 1 SV = 2 | | |
| Q9VL70 | CG4600 | yip2 | HL08109p OS = Drosophila | High | Not |
| | | | melanogaster GN = yip2 PE = 2 SV = 1 | | Found |
| Q9VLG3 | CG18088 | | CG18088, isoform A OS = Drosophila | High | Not |
| | | | melanogaster GN = CG18088-RA | | Found |
| | | | PE = 2 SV = 1 | | |
| Q9VXH7 | CG9917 | | CG9917, isoform A OS = Drosophila | Not | High |
| | | | melanogaster GN = CG9917-RA | Found | |
| | | | PE = 2 SV = 1 | | |
| Q9W373 | CG12119 | SmydA-9 | CG12119 OS = Drosophila | Not | High |
| | | | melanogaster GN = CG12119 PE = 4 | Found | |
| | | | SV = 1 | | |
| Q7K110 | CG6370 | OstΔ | CG6370 OS = Drosophila | High | Not |
| | | | melanogaster GN = OstDelta PE = 2 | | Found |
| | | | SV = 1 | | |
| Q9VJ59 | CG10373 | Jwa | Jwa ortholog (Fragment) | High | Not |
| | | | OS = Drosophila melanogaster | | Found |
| | | | GN = Jwa PE = 4 SV = 4 | | |
| Q9VEB1 | CG7998 | Mdh2 | CG7998 OS = Drosophila | High | Not |
| | | | melanogaster GN = Mdh2 PE = 2 | | Found |
| | | | SV = 1 | | |
| Q9VKM7 | CG33129 | | AT01533p OS = Drosophila | High | Not |
| | | | melanogaster GN = CG6089 PE = 2 | | Found |
| | | | SV = 2 | | |
| A1ZBJ2 | CG7461 | | CG7461 OS = Drosophila | High | Not |
| | | | melanogaster GN = CG7461 PE = 3 | | Found |
| | | | SV = 2 | | |
| P45594 | CG4254 | tsr | Cofilin/actin-depolymerizing factor | High | Not |
| | | | homolog OS = Drosophila | | Found |
| | | | melanogaster GN = tsr PE = 2 SV = 1 | | |
| Q9V491 | CG11081 | PlexA | Plexin A, isoform A OS = Drosophila | High | Not |
| | | | melanogaster GN = plexA PE = 4 | | Found |
| | | | SV = 1 | | |
| Q9VL69 | CG5885 | | CG5885 OS = Drosophila | High | Not |
| | | | melanogaster GN = BEST:CK01296 | | Found |
| | | | PE = 2 SV = 1 | | |
| Q8SXQ1 | CG9629 | Aldh7A1 | CG9629 OS = Drosophila | High | Not |
| | | | melanogaster GN = CG9629 PE = 2 | | Found |
| | | | SV = 1 | | |
| Q9VG64 | CG18549 | | CG18549 OS = Drosophila | High | Not |
| | | | melanogaster GN = CG18549 PE = 2 | | Found |
| | | | SV = 2 | | |
| Q9VXZ3 | CG32594 | be | Ben, isoform C OS = Drosophila | High | Not |
| | | | melanogaster GN = be PE = 2 SV = 2 | | Found |
| Q9VHM2 | CG8202 | | UPF0505 protein CG8202 | Not | High |
| | | | OS = Drosophila melanogaster | Found | |
| | | | GN = CG8202 PE = 2 SV = 3 | | |
| Q9VFS8 | CG9588 | | 26S proteasome non-ATPase | Not | High |
| | | | regulatory subunit 9 OS = Drosophila | Found | |
| | | | melanogaster GN = CG9588 PE = 1 | | |
| | | | SV = 1 | | |
| Q9VUE4 | CG3868 | | CG3868 OS = Drosophila | High | Not |
| | | | melanogaster GN = CG3868 PE = 4 | | Found |
| | | | SV = 1 | | |
| Q0E8E8 | CG4994 | Mpcp2 | MIP08013p1 OS = Drosophila | High | Not |
| | | | melanogaster GN = Mpcp PE = 2 | | Found |
| | | | SV = 1 | | |
| Q9VUZ0 | CG5474 | SsRβ | BcDNA.LD10457 OS = Drosophila | High | Not |
| | | | melanogaster GN = SsRbeta PE = 2 | | Found |
| | | | SV = 2 | | |

To specifically purify the dsRNA receptor(s) the inventors developed a state-of-the-art immunoprecipitation protocol based on the CLIP assay (CrossLinking and Immunoprecipitation) (FIG. 2d). The inventors used Dynasore to inhibit the internalization of dsRNA-Cy3 by S2Xpress cells, thereby sequestering a high concentration of dsRNA-Cy3 at the cell surface. Next, the inventors UV-crosslinked dsRNA-Cy3 to interacting proteins and performed an immunoprecipitation (IP) using an anti-Cy3 antibody that the inventors found to have high specificity for dsRNA-Cy3 (FIG. 7b). LC-MS/MS analysis of proteins purified with anti-Cy3 antibody allowed the inventors' to identify 79 proteins (FIG. 2e, Table 2). Of these proteins, 13 were also found on the surface of S2Xpress cells, and only 2 on the surface of both cell types (S2naive and S2Xpress). Cellular component analysis showed that several of the identified proteins corresponded to mitochondrion proteins and membrane proteins (FIG. 2f) as well as several nuclear proteins. This could be explained by the fact that dsRNA added to the medium seemed to bind to some dying cells present during the incubation (FIG. 7c). When examining the molecular functions of the identified proteins, the inventors found a high percentage of RNA binding proteins, indicating that the IP protocol was successful in pulling-down proteins binding to dsRNA.

Together, these two proteomic approaches allowed the inventors to confirm that there are differences in the composition of the cell surface of S2naive and S2Xpress cells, and to obtain a list of potential dsRNA receptor(s) candidate proteins.

TABLE 2
List of proteins found in dsRNA binding proteins immunoprecipitation (FIG. 2d-f)

| | | | | Replicate | Replicate |
|---|---|---|---|---|---|
| Accession | Gene | Gene Name | Description | 1 | 2 |
| Q9VBU7 | CG11856 | Nup358 | Nup358 OS = Drosophila | High | High |
| | | | melanogaster | | |
| | | | GN = Nup358 PE = 4 SV = 2 | | |
| A8DZ29 | CG10811 | eIF4G1 | Eukaryotic translation | Not Found | High |
| | | | initiation factor 4G, | | |
| | | | isoform B OS = Drosophila | | |
| | | | melanogaster GN = eIF4G | | |
| | | | PE = 4 SV = 1 | | |
| Q9VJJ8 | CG42389 | mtgo | CG42389, isoform G | Not Found | High |
| | | | OS = Drosophila | | |
| | | | melanogaster | | |
| | | | GN = CG4668 PE = 2 SV = 2 | | |
| P11147 | CG4264 | Hsc70-4 | Heat shock 70 kDa | High | High |
| | | | protein cognate 4 | | |
| | | | OS = Drosophila | | |
| | | | melanogaster | | |
| | | | GN = Hsc70-4 PE = 1 SV = 3 | | |
| A1Z6H7 | CG7897 | Gp210 | Gp210 OS = Drosophila | Not Found | High |
| | | | melanogaster | | |
| | | | GN = Gp210 PE = 4 SV = 1 | | |
| Q7K110 | CG6370 | OstΔ | CG6370 OS = Drosophila | Not Found | High |
| | | | melanogaster | | |
| | | | GN = OstDelta PE = 2 | | |
| | | | SV = 1 | | |
| Q9VHR5 | CG9684 | | CG9684 OS = Drosophila | Not Found | High |
| | | | melanogaster | | |
| | | | GN = CG9684 PE = 4 SV = 2 | | |
| A8DY81 | CG34407 | Not1 | Not1, isoform C | Not Found | High |
| | | | OS = Drosophila | | |
| | | | melanogaster GN = Not1 | | |
| | | | PE = 1 SV = 1 | | |
| P29845 | CG8542 | Hsc70-5 | Heat shock 70 kDa | High | High |
| | | | protein cognate 5 | | |
| | | | OS = Drosophila | | |
| | | | melanogaster | | |
| | | | GN = Hsc70-5 PE = 1 SV = 2 | | |
| Q9VHP0 | CG9748 | bel | ATP-dependent RNA | High | High |
| | | | helicase bel | | |
| | | | OS = Drosophila | | |
| | | | melanogaster GN = bel | | |
| | | | PE = 1 SV = 1 | | |
| Q8IQQ0 | CG11661 | Ogdh | Neural conserved at | High | High |
| | | | 73EF, isoform F | | |
| | | | OS = Drosophila | | |
| | | | melanogaster | | |
| | | | GN = Nc73EF PE = 2 SV = 1 | | |
| P29742 | CG9012 | Chc | Clathrin heavy chain | Not Found | High |
| | | | OS = Drosophila | | |
| | | | melanogaster GN = Chc | | |
| | | | PE = 1 SV = 1 | | |
| Q7KN75 | CG5170 | Dp1 | Dodeca-satellite-binding | Not Found | High |
| | | | protein 1, isoform A | | |
| | | | OS = Drosophila | | |
| | | | melanogaster GN = Dp1 | | |
| | | | PE = 2 SV = 1 | | |
| Q9VWH4 | CG12233 | Idh3a | Probable isocitrate | High | High |
| | | | dehydrogenase [NAD] | | |
| | | | subunit alpha, | | |
| | | | mitochondrial | | |
| | | | OS = Drosophila | | |
| | | | melanogaster | | |
| | | | GN = I(1)G0156 PE = 2 | | |
| | | | SV = 1 | | |
| P06605 | CG2512 | αTub84D | Tubulin alpha-3 chain | High | High |
| | | | OS = Drosophila | | |
| | | | melanogaster | | |
| | | | GN = alphaTub84D PE = 2 | | |
| | | | SV = 1 | | |
| P41042 | CG11276 | RpS4 | 40S ribosomal protein S4 | Not Found | High |
| | | | OS = Drosophila | | |
| | | | melanogaster GN = RpS4 | | |
| | | | PE = 1 SV = 2 | | |
| P21187 | CG5119 | pAbp | Polyadenylate-binding | High | High |
| | | | protein OS = Drosophila | | |
| | | | melanogaster GN = pAbp | | |
| | | | PE = 1 SV = 3 | | |
| E1JIJ7 | CG7518 | | CG7518, isoform C | Not Found | High |
| | | | OS = Drosophila | | |
| | | | melanogaster | | |
| | | | GN = CG7518 PE = 4 SV = 1 | | |
| M9PJN0 | CG12047 | mud | Mushroom body defect, | Not Found | High |
| | | | isoform K OS = Drosophila | | |
| | | | melanogaster GN = mud | | |
| | | | PE = 4 SV = 1 | | |
| Q9VJ19 | CG10652 | RpL30 | RE25263p | High | High |
| | | | OS = Drosophila | | |
| | | | melanogaster | | |
| | | | GN = RpL30 PE = 1 SV = 1 | | |
| Q7K5K3 | CG11876 | Pdhb | CG11876, isoform A | High | High |
| | | | OS = Drosophila | | |
| | | | melanogaster | | |
| | | | GN = CG11876 PE = 2 | | |
| | | | SV = 1 | | |
| Q9VL70 | CG4600 | yip2 | HL08109p | High | High |
| | | | OS = Drosophila | | |
| | | | melanogaster GN = yip2 | | |
| | | | PE = 2 SV = 1 | | |
| Q9V9W7 | CG1569 | rod | Rough deal | Not Found | High |
| | | | OS = Drosophila | | |
| | | | melanogaster GN = rod | | |
| | | | PE = 4 SV = 2 | | |
| Q7KW39 | CG17896 | | Probable | High | High |
| | | | methylmalonate- | | |
| | | | semialdehyde | | |
| | | | dehydrogenase | | |
| | | | [acylating], mitochondrial | | |
| | | | OS = Drosophila | | |
| | | | melanogaster | | |
| | | | GN = CG17896 PE = 2 | | |
| | | | SV = 1 | | |
| Q8T4C9 | CG6310 | | AT03632p | Not Found | High |
| | | | OS = Drosophila | | |
| | | | melanogaster | | |
| | | | GN = CG6310 PE = 2 SV = 1 | | |
| Q7KN94 | CG8996 | wal | Walrus, isoform A | High | High |
| | | | OS = Drosophila | | |
| | | | melanogaster GN = wal | | |
| | | | PE = 2 SV = 1 | | |
| M9PCQ6 | CG6509 | Dlg5 | CG6509, isoform C | Not Found | High |
| | | | OS = Drosophila | | |
| | | | melanogaster | | |
| | | | GN = CG6509 PE = 4 SV = 1 | | |
| P08841 | CG3401 | βTub60D | Tubulin beta-3 chain | Not Found | High |
| | | | OS = Drosophila | | |
| | | | melanogaster | | |
| | | | GN = betaTub60D PE = 2 | | |
| | | | SV = 2 | | |
| Q9VV75 | CG4169 | UQCR-C2 | AT02348p | High | High |
| | | | OS = Drosophila | | |
| | | | melanogaster | | |
| | | | GN = CG4169 PE = 2 SV = 1 | | |
| Q9W1G7 | CG5330 | Nap1 | LD21576p | Not Found | High |
| | | | OS = Drosophila | | |
| | | | melanogaster GN = Nap1 | | |
| | | | PE = 2 SV = 1 | | |
| P22700 | CG3725 | SERCA | Calcium-transporting | Not Found | High |
| | | | ATPase | | |
| | | | sarcoplasmic/endoplasmic | | |
| | | | reticulum type | | |
| | | | OS = Drosophila | | |
| | | | melanogaster GN = Ca- | | |
| | | | P60A PE = 1 SV = 2 | | |
| O01666 | CG7610 | ATPsynγ | ATP synthase subunit | High | High |
| | | | gamma, mitochondrial | | |
| | | | OS = Drosophila | | |
| | | | melanogaster | | |
| | | | GN = ATPsyn-gamma | | |
| | | | PE = 2 SV = 2 | | |
| Q9VD58 | CG6439 | Idh3b | CG6439, isoform A | Not Found | High |
| | | | OS = Drosophila | | |
| | | | melanogaster | | |
| | | | GN = CG6439 PE = 2 SV = 1 | | |
| P17336 | CG6871 | Cat | Catalase OS = Drosophila | High | High |
| | | | melanogaster GN = Cat | | |
| | | | PE = 1 SV = 2 | | |
| Q8IQV9 | CG11943 | Nup205 | Nucleoporin 205, isoform | Not Found | High |
| | | | B OS = Drosophila | | |
| | | | melanogaster | | |
| | | | GN = Nup205 PE = 4 SV = 1 | | |
| Q9VEZ3 | CG5000 | msps | LP04448p | Not Found | High |
| | | | OS = Drosophila | | |
| | | | melanogaster GN = msps | | |
| | | | PE = 1 SV = 3 | | |
| Q9V397 | CG4389 | Mtpα | BcDNA.GH12558 | High | High |
| | | | OS = Drosophila | | |
| | | | melanogaster | | |
| | | | GN = Mtpalpha PE = 2 | | |
| | | | SV = 1 | | |
| Q960Z0 | CG1453 | Klp10A | Kinesin-like protein | Not Found | High |
| | | | Klp10A OS = Drosophila | | |
| | | | melanogaster | | |
| | | | GN = Klp10A PE = 1 SV = 1 | | |
| M9PFK7 | CG7439 | AGO2A | Argonaute 2, isoform E | Not Found | High |
| | | | OS = Drosophila | | |
| | | | melanogaster GN = AGO2 | | |
| | | | PE = 4 SV = 1 | | |
| P14130 | CG14524 | | 40S ribosomal protein | High | High |
| | | | S14 OS = Drosophila | | |
| | | | melanogaster | | |
| | | | GN = RpS14a PE = 1 SV = 1 | | |
| P23128 | CG4916 | me31B | Putative ATP-dependent | Not Found | High |
| | | | RNA helicase me31b | | |
| | | | OS = Drosophila | | |
| | | | melanogaster | | |
| | | | GN = me31B PE = 1 SV = 3 | | |
| A1ZBJ2 | CG7461 | | CG7461 OS = Drosophila | High | High |
| | | | melanogaster | | |
| | | | GN = CG7461 PE = 3 SV = 2 | | |
| Q94522 | CG1065 | Scsα1 | Succinyl-CoA ligase | Not Found | High |
| | | | [ADP/GDP-forming] | | |
| | | | subunit alpha, | | |
| | | | mitochondrial | | |
| | | | OS = Drosophila | | |
| | | | melanogaster | | |
| | | | GN = Scsalpha PE = 2 | | |
| | | | SV = 3 | | |
| Q9VSA3 | CG12262 | Mcad | Probable medium-chain | High | High |
| | | | specific acyl-CoA | | |
| | | | dehydrogenase, | | |
| | | | mitochondrial | | |
| | | | OS = Drosophila | | |
| | | | melanogaster | | |
| | | | GN = CG12262 PE = 1 | | |
| | | | SV = 1 | | |
| P84040 | CG31611 | His4:CG31611 | Histone H4 | High | High |
| | (and many | | OS = Drosophila | | |
| | others) | | melanogaster GN = His4 | | |
| | | | PE = 1 SV = 2 | | |
| Q0E8E8 | CG4994 | Mpcp2 | MIP08013p1 | Not Found | High |
| | | | OS = Drosophila | | |
| | | | melanogaster GN = Mpcp | | |
| | | | PE = 2 SV = 1 | | |
| P24156 | CG10691 | Phb1 | Protein I(2)37Cc | High | High |
| | | | OS = Drosophila | | |
| | | | melanogaster | | |
| | | | GN = I(2)37Cc PE = 2 | | |
| | | | SV = 2 | | |
| Q24151 | CG4257 | Stat92E | Signal transducer and | Not Found | High |
| | | | transcription activator | | |
| | | | OS = Drosophila | | |
| | | | melanogaster | | |
| | | | GN = Stat92E PE = 1 SV = 1 | | |
| M9PI41 | CG10522 | sti | Sticky, isoform B | Not Found | High |
| | | | OS = Drosophila | | |
| | | | melanogaster GN = sti | | |
| | | | PE = 4 SV = 1 | | |
| Q9VSL8 | CG6831 | rhea | Rhea, isoform B | Not Found | High |
| | | | OS = Drosophila | | |
| | | | melanogaster GN = rhea | | |
| | | | PE = 1 SV = 1 | | |
| Q9VZ23 | CG1404 | Ran | GTP-binding nuclear | Not Found | High |
| | | | protein Ran | | |
| | | | OS = Drosophila | | |
| | | | melanogaster GN = ran | | |
| | | | PE = 1 SV = 1 | | |
| M9PCF8 | CG32138 | Frl | CG32138, isoform D | Not Found | High |
| | | | OS = Drosophila | | |
| | | | melanogaster | | |
| | | | GN = CG32138 PE = 4 | | |
| | | | SV = 1 | | |
| Q9VB46 | CG3373 | Hmu | FI18644p1 | Not Found | High |
| | | | OS = Drosophila | | |
| | | | melanogaster GN = Hmu | | |
| | | | PE = 2 SV = 1 | | |
| A8JQV8 | CG43140 | pyd | Polychaetoid, isoform H | Not Found | High |
| | | | OS = Drosophila | | |
| | | | melanogaster GN = pyd | | |
| | | | PE = 4 SV = 1 | | |
| Q9VB10 | CG5590 | Hsdl2 | CG5590 OS = Drosophila | Not Found | High |
| | | | melanogaster | | |
| | | | GN = CG5590 PE = 2 SV = 1 | | |
| P32100 | CG4897 | RpL7 | 60S ribosomal protein L7 | High | High |
| | | | OS = Drosophila | | |
| | | | melanogaster GN = RpL7 | | |
| | | | PE = 1 SV = 2 | | |
| Q9VDT1 | CG4703 | Arc42 | Arc42 OS = Drosophila | High | High |
| | | | melanogaster GN = Arc42 | | |
| | | | PE = 2 SV = 1 | | |
| P48149 | CG2033 | RpS15Aa | 40S ribosomal protein | Not Found | High |
| | | | S15Aa OS = Drosophila | | |
| | | | melanogaster | | |
| | | | GN = RpS15Aa PE = 1 | | |
| | | | SV = 2 | | |
| Q9XZ11 | CG7139 | | CG7139, isoform A | Not Found | High |
| | | | OS = Drosophila | | |
| | | | melanogaster | | |
| | | | GN = CG7139 PE = 2 SV = 1 | | |
| A1Z9I5 | CG13345 | tum | Tumbleweed | Not Found | High |
| | | | OS = Drosophila | | |
| | | | melanogaster GN = tum | | |
| | | | PE = 4 SV = 1 | | |
| A1Z6H6 | CG7791 | | CG7791 OS = Drosophila | Not Found | High |
| | | | melanogaster | | |
| | | | GN = CG7791 PE = 3 SV = 1 | | |
| Q0KIE7 | CG1651 | Ank | Ankyrin, isoform B | Not Found | High |
| | | | OS = Drosophila | | |
| | | | melanogaster GN = Ank | | |
| | | | PE = 2 SV = 1 | | |
| Q9TVM2 | CG13387 | emb | Exportin-1 | Not Found | High |
| | | | OS = Drosophila | | |
| | | | melanogaster GN = emb | | |
| | | | PE = 1 SV = 1 | | |
| M9PI44 | CG10686 | tral | Trailer hitch, isoform F | Not Found | High |
| | | | OS = Drosophila | | |
| | | | melanogaster GN = tral | | |
| | | | PE = 4 SV = 1 | | |
| Q9VCA8 | CG33106 | mask | Ankyrin repeat and KH | Not Found | High |
| | | | domain-containing | | |
| | | | protein mask | | |
| | | | OS = Drosophila | | |
| | | | melanogaster GN = mask | | |
| | | | PE = 1 SV = 2 | | |
| P46223 | CG3314 | RpL7A | 60S ribosomal protein | Not Found | High |
| | | | L7a OS = Drosophila | | |
| | | | melanogaster | | |
| | | | GN = RpL7A PE = 1 SV = 2 | | |
| Q9VAC1 | CG7920 | | CG7920, isoform A | Not Found | High |
| | | | OS = Drosophila | | |
| | | | melanogaster | | |
| | | | GN = CG7920 PE = 2 SV = 1 | | |
| Q7KVX1 | CG7010 | Pdha | Lethal (1) G0334, isoform | High | High |
| | | | C OS = Drosophila | | |
| | | | melanogaster | | |
| | | | GN = I(1)G0334 PE = 2 | | |
| | | | SV = 1 | | |
| Q7KTI8 | CG8222 | Pvr | PDGF-and VEGF- | Not Found | High |
| | | | receptor related, isoform | | |
| | | | E OS = Drosophila | | |
| | | | melanogaster GN = Pvr | | |
| | | | PE = 4 SV = 1 | | |
| A1ZAN6 | CG9635 | RhoGEF2 | RhoGEF2, isoform D | Not Found | High |
| | | | OS = Drosophila | | |
| | | | melanogaster | | |
| | | | GN = RhoGEF2 PE = 4 | | |
| | | | SV = 1 | | |
| A8JRB1 | CG10618 | CHKov1 | CHKov1, isoform F | Not Found | High |
| | | | OS = Drosophila | | |
| | | | melanogaster | | |
| | | | GN = CHKov1 PE = 4 SV = 1 | | |
| Q8MLY8 | CG7808 | RpS8 | 40S ribosomal protein S8 | Not Found | High |
| | | | OS = Drosophila | | |
| | | | melanogaster GN = RpS8 | | |
| | | | PE = 1 SV = 1 | | |
| B7Z0E2 | CG7015 | Unr | FI18812p1 | Not Found | High |
| | | | OS = Drosophila | | |
| | | | melanogaster GN = Unr | | |
| | | | PE = 2 SV = 1 | | |
| Q7K511 | CG3835 | D2hgdh | CG3835, isoform A | Not Found | High |
| | | | OS = Drosophila | | |
| | | | melanogaster | | |
| | | | GN = EG:87B1.3 PE = 2 | | |
| | | | SV = 1 | | |
| Q9VZL3 | CG10849 | Sc2 | Sc2 OS = Drosophila | Not Found | High |
| | | | melanogaster GN = Sc2 | | |
| | | | PE = 4 SV = 1 | | |
| A1Z8P9 | CG8274 | Mtor | Nucleoprotein TPR | Not Found | High |
| | | | OS = Drosophila | | |
| | | | melanogaster GN = Mtor | | |
| | | | PE = 1 SV = 1 | | |
| Q9VKD3 | CG12264 | Nfs1 | Probable cysteine | High | High |
| | | | desulfurase, | | |
| | | | mitochondrial | | |
| | | | OS = Drosophila | | |
| | | | melanogaster | | |
| | | | GN = CG12264 PE = 2 | | |
| | | | SV = 1 | | |
| Q9W0B8 | CG7961 | αCOP | Alpha-coatomer protein, | Not Found | High |
| | | | isoform A OS = Drosophila | | |
| | | | melanogaster | | |
| | | | GN = alphaCop PE = 2 | | |
| | | | SV = 1 | | |
| Q86B79 | CG4620 | unk | RING finger protein | Not Found | High |
| | | | unkempt OS = Drosophila | | |
| | | | melanogaster GN = unk | | |
| | | | PE = 1 SV = 1 | | |

### Example 3: a Functional Screen of Candidate dsRNA Receptors Identified Hsc70-4 as a Protein with a Role in dsRNA Internalization

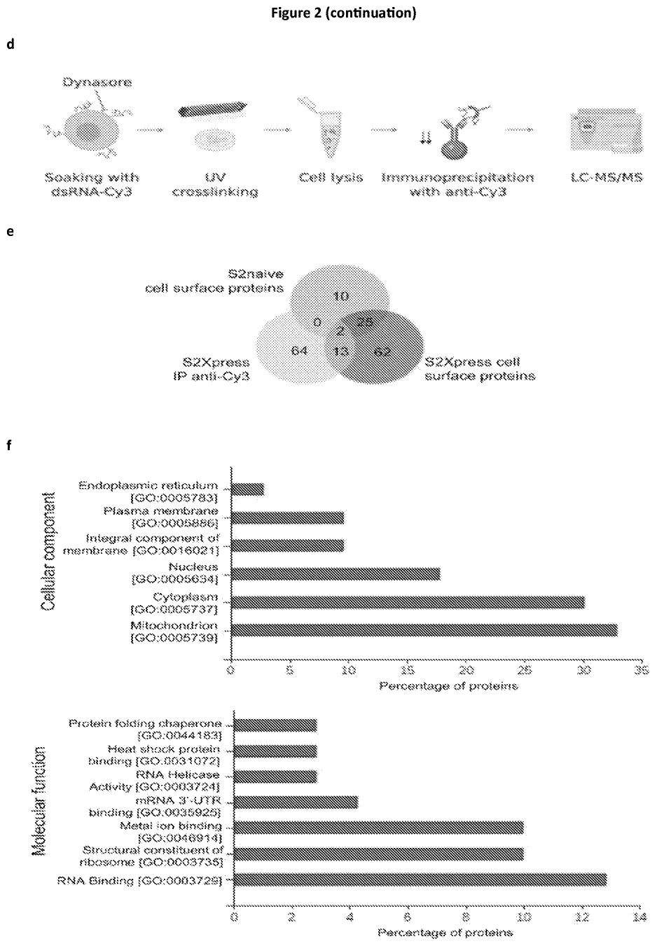

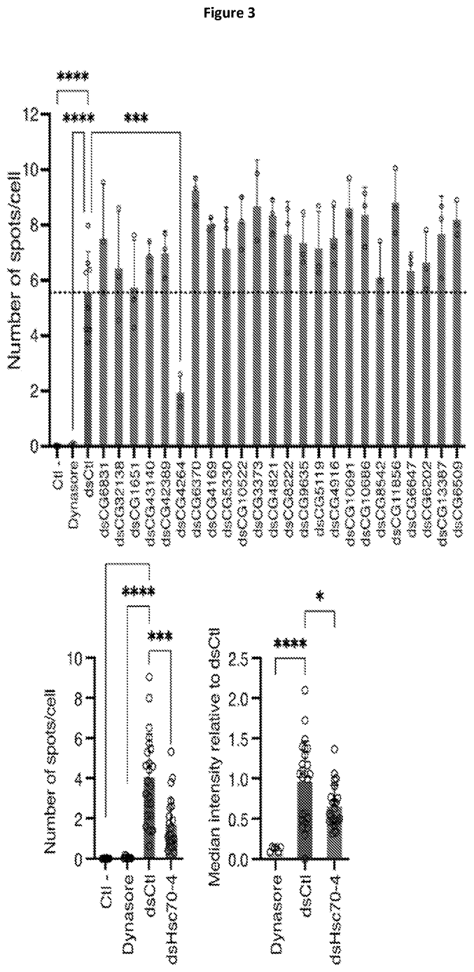

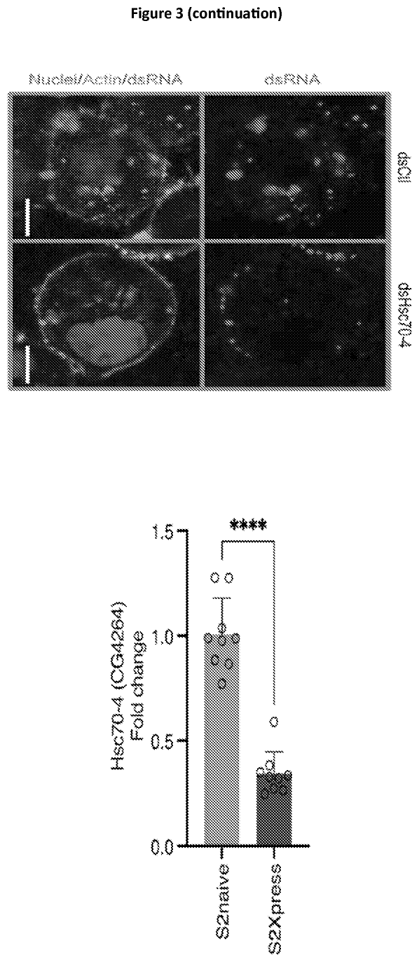

The inventors next performed an in silico analysis of the proteins identified by the inventors' two proteomic approaches to select candidates to test in S2Xpress cells. As a selection strategy, the inventors considered protein localization, type of protein, and presence in the results of both proteomic strategies. Through this process, the inventors produced a list of 24 candidate proteins to test for their possible role as a dsRNA receptor (FIG. 3a).

| Treatment | p-value vs. dsCtl |
|---|---|
| Ctl - | <0.0001 |
| Dynasore | <0.0001 |
| dsCG6831 | 0.2354 |
| dsCG32138 | 0.9936 |
| dsCG1651 | 0.9997 |
| dsCG43140 | 0.8198 |
| dsCG42389 | 0.7233 |
| dsCG4264 | 0.0003 |
| dsCG6370 | 0.0002 |
| dsCG4169 | 0.0451 |
| dsCG5330 | 0.5242 |
| dsCG10522 | 0.0338 |
| dsCG3373 | 0.0209 |
| dsCG4621 | 0.0126 |
| dsCG8222 | 0.1595 |
| dsCG9635 | 0.3443 |
| dsCG5119 | 0.5290 |
| dsCG4916 | 0.2125 |
| dsCG10691 | 0.0043 |
| dsCG10686 | 0.0120 |
| dsCG8542 | 0.9992 |
| dsCG11856 | 0.0018 |
| dsCG6647 | 0.9949 |
| dsCG6202 | 0.9549 |
| dsCG13387 | 0.1456 |
| dsCG6509 | 0.0230 |

This table shows specific p-values of panel (a) from FIG. 3.

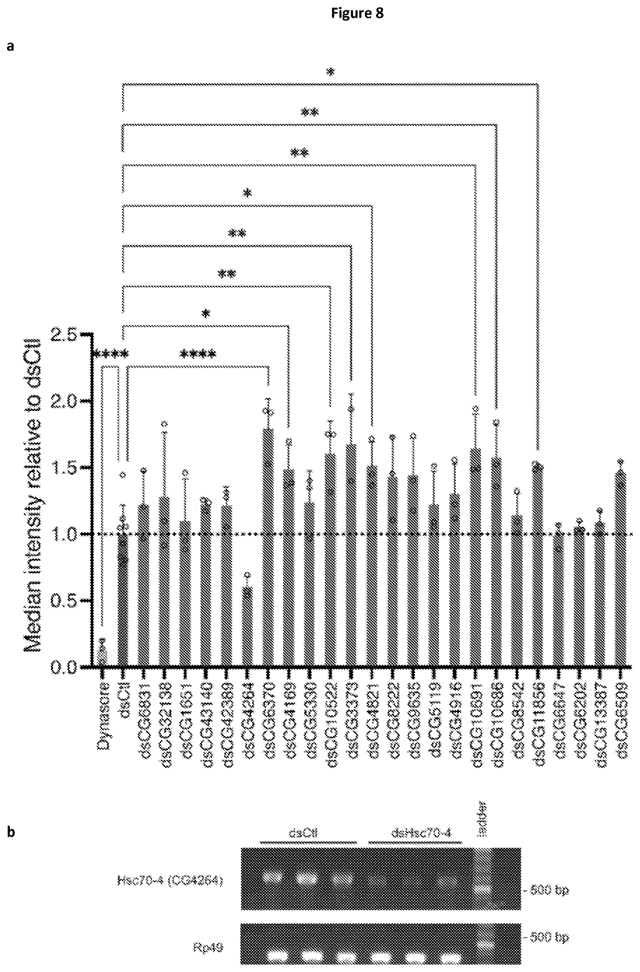

To this end, the inventors performed a high-content imaging screen based on silencing candidate proteins by transfection with candidate-gene-specific dsRNA followed by incubation with nonspecific dsRNA-Cy3. The inventors quantified both total Cy3 intensity and number of Cy3 spots inside the cytoplasm as an indicator of dsRNA-Cy3 internalization. The inventors successfully identified one candidate, CG4264 (also known as Hsc70-4), that impaired the internalization of dsRNA when silenced (FIG. 3a-b, FIG. 8a). The inventors confirmed that silencing of Hsc70-4 was efficient by amplification of the full-length dsRNA target region within the Hsc70-4 transcript by RT-PCR (FIG. 8c). Here, RT-PCR band intensity was much weaker for cells treated with dsHsc70-4 compared to cells treated with dsCtl. Furthermore, confocal imaging analysis confirmed that when Hsc70-4 was silenced, there was a decrease in the internalization of dsRNA-Cy3 (FIG. 3c). Notably, the inventors' proteomics assays identified Hsc70-4 as both a dsRNA-binding protein and a cell surface protein in S2Xpress cells, but not in S2naive cells. Surprisingly, when the inventors compared the expression of Hsc70-4 between S2naive and S2Xpress cells by RT-qPCR, the inventors found that S2naive cells had significantly greater expression than S2Xpress cells (FIG. 3d). Nevertheless, this result shows mRNA levels and does not necessarily reflect protein levels. Overall, these results highlight Hsc70-4 as a key factor in dsRNA internalization in S2Xpress cells and suggest that the difference between S2naive cells and S2Xpress cells regarding Hsc70-4 could relate to its localization, binding partners, or post-translational modifications.

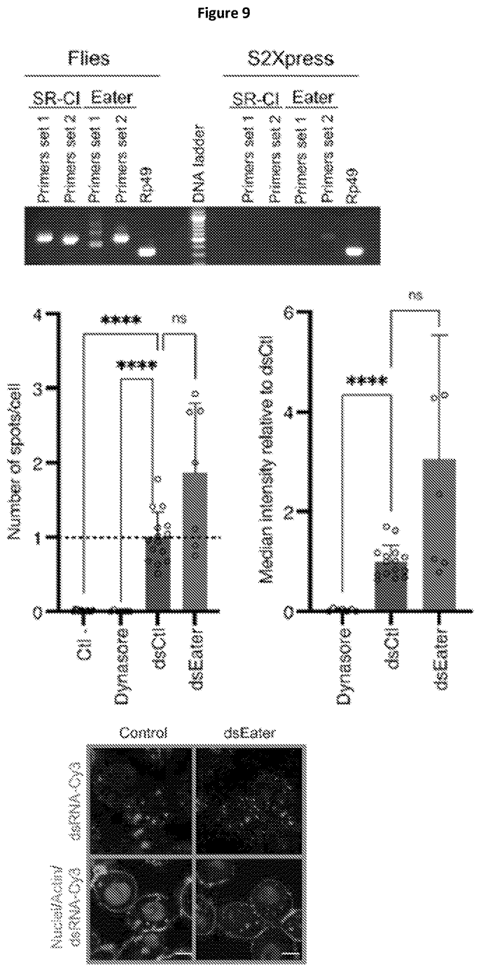

Of note, two scavenger receptors, SR-CI and Eater, have been previously described to mediate the internalization of dsRNA in S2 cells¹⁹. Although neither of these proteins appeared in the inventors' proteomic assays, the inventors tested if they affected internalization in the inventors' model. The inventors were not able to produce dsRNA corresponding to SR-CI since the inventors could not obtain an RT-PCR product from S2Xpress RNA (FIG. 9a). This precluded the inventors' from performing the inventors' dsRNA-based silencing assay for SR-CI and indicates that SR-CI is not expressed in S2Xpress cells. The inventors were able to produce dsRNA corresponding to Eater, but the inventors didn't see an effect on dsRNA internalization in S2xpress cells when the inventors silenced this protein and tested internalization by High-content fluorescence quantification and confocal imaging. (FIG. 9b-c).

### Example 4: Membrane Localization of rHsc70-4

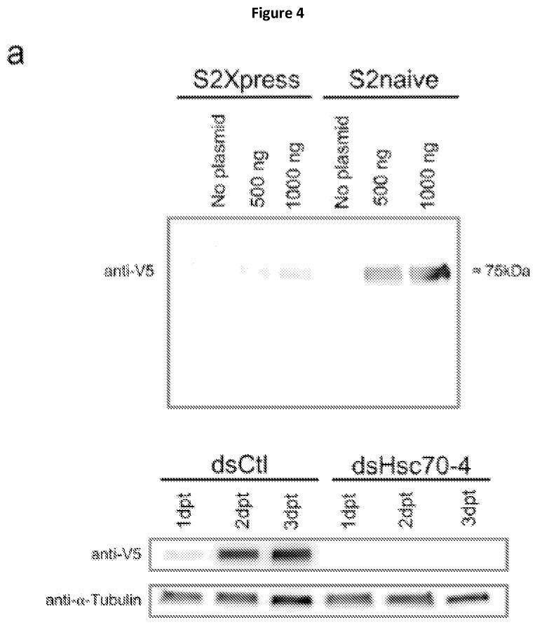

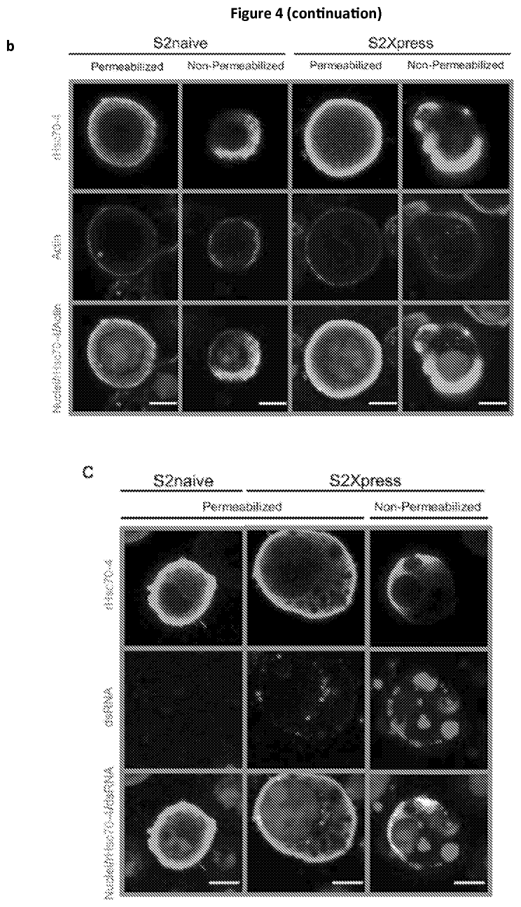

To better understand the role of Hsc70-4 as a mediator of dsRNA internalization, the inventors cloned the protein fused to a V5 tag in a Drosophila expression plasmid (hereafter referred to as pHsc70-4). This protein, referred to as recombinant Hsc70-4 (rHsc70-4), had the expected molecular size (FIG. 4a). Furthermore, when S2Xpress cells were co-transfected with pHsc70-4 and dsHsc70-4, the inventors were not able to detect the recombinant protein, indicating that the sequence was correct and that silencing of rHsc70-4 by dsHsc70-4 was effective. Next, the inventors transfected S2naive and S2Xpress cells with pHsc70-4 to determine the localization of rHsc70-4. The inventors found that localization was similar in both cell types, with some cytoplasmic localization and mostly with accumulation at the cell edges (FIG. 4b).

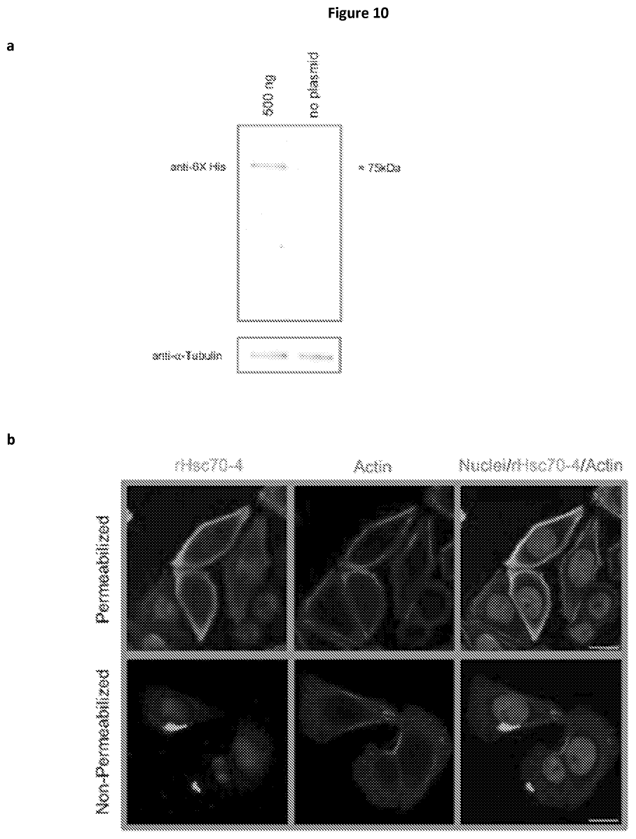

To determine if rHsc70-4 was present at the cell surface, the inventors performed non-permeabilization assays. These assays rely on the inability of antibodies to pass through the plasma membrane of non-permeabilized cells during the treatment. Therefore, immunofluorescence signals obtained from non-permeabilized cells are an indication that the target protein is present at the cell surface. The inventors' results showed that rHsc70-4 was located at the cell surface, with an extracellular face in both S2naive and S2Xpress cells (FIG. 4b). Despite the cell surface localization of rHsc70-4 in in S2naive cells, expression of this protein was not sufficient to induce internalization of dsRNA (FIG. 4c). This is not surprising since the inventors have previously found that S2naive cells do express Hsc70-4. Notably, the cell membrane localization of rHsc70-4 was preserved in mammalian cells, suggesting a conserved role for this protein at the cell surface (FIG. 10a-b). Hsc70-4 does not present predictable transmembrane domain(s). Its localization at the cell surface suggests that this is a peripheral membrane protein that anchors to the cell membrane by interacting with the lipid bilayer. The inventors' results confirm that Hsc70-4 is in fact present at the cell surface and thus could be acting as a cell surface receptor or co-receptor for dsRNA.

### Example 5: rHsc70-4 Binds dsRNA In Vitro

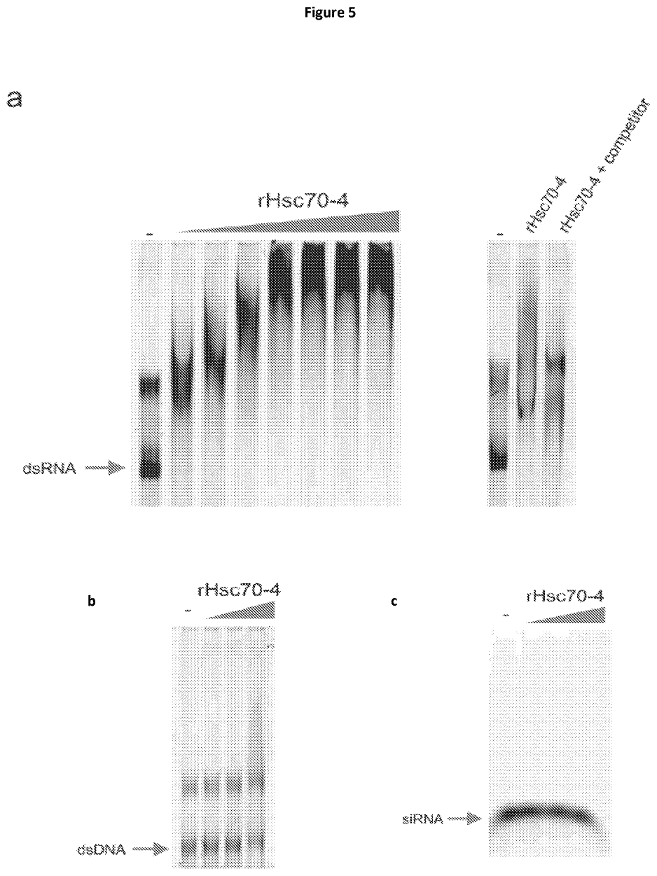

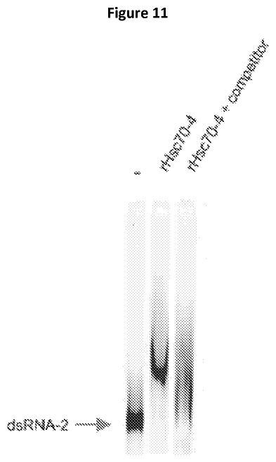

Several different domains have been identified as dsRNA binding domains in proteins, including the αβββα fold commonly known as dsRNA binding domain (dsRBD), the helicase domain, and the nucleotidyltransferase (NTase) domain^{28,29}. Hsc70-4, does not present a previously identified dsRNA binding domain. Thus, the inventors next tested if rHsc70-4 can bind dsRNA in vitro. rHsc70-4 was commercially produced by expression of recombinant protein in E. coli and followed by His-tag purification. Purified rHsc70-4 was incubated at different concentrations with dsRNA-Cy3 and binding of rHsc70-4 to dsRNA-Cy3 was assessed by electrophoretic mobility shift assays (EMSAs). The inventors found that rHsc70-4 binds dsRNA in vitro, with increasing concentrations of rHsc70-4 resulting in decreased mobility of dsRNA-Cy3 compared to dsRNA-Cy3 alone (FIG. 5a). Moreover, the fact that the electrophoretic shift gradually increased with increasing concentrations of rHsc70-4 until reaching a full shift may indicate that more than one molecule of rHsc70-4 can bind to each molecule of dsRNA-Cy3. A competitor assay with unlabeled dsRNA was performed to confirm binding specificity. Here, the inventors observed increased mobility for dsRNA-Cy3 in the presence of competitor dsRNA compared to the assay without competitor dsRNA, indicating that unlabeled dsRNA displaced labeled dsRNA (FIG. 5a). Furthermore, by using a dsRNA with a different sequence as probe, the inventors confirmed that binding was not sequence-dependent (FIG. 11). Moreover, the electrophoretic mobility of dsDNA-Cy3 or siRNA-Cy3 was not altered by incubation with rHsc70-4 (FIG. 5b&c). These results confirm that rHsc70-4 can bind dsRNA in vitro, and that this binding is specific for dsRNA.

### Materials & Methods

### Cells, Plasmids and Antibodies

Drosophila Schneider 2 (S2naive) cells (Invitrogen), S2R+²⁴ (Drosophila Genomics Resource Center), S2p FHV and S2p FHV+DAV²⁷ were cultured at 25° C. in Schneider's Insect Medium (GIBCO), supplemented with 10% heat-inactivated fetal bovine serum (GIBCO), 2 mM L-glutamine (GIBCO), 100 U/ml penicillin (GIBCO) and 100 mg/ml streptomycin (GIBCO). S2Xpress cells were cultured in Insect-XPRESS™ protein-free medium (LONZA, Belgium) supplemented with 100 U/ml penicillin and 100 mg/ml streptomycin (GIBCO). S2naive and S2Xpress cells were checked by next-generation sequencing of small RNAs for infection with CrPv, DAV, DCV, DXV, FHV, Nora virus, Sigma virus, American nodavirus, and Drosophila birnavirus. Hela cells (ATCC) were cultured at 37° C. with 5% CO₂ in DMEM+GlutaMAX medium (GIBCO) supplemented with 10% heat-inactivated fetal bovine serum, 2 mM L-glutamine, 100 U/ml penicillin and 100 mg/ml streptomycin. pMT/V5-HisB (Invitrogen) expressing either Firefly or Renilla Luciferase under the control of a copper-inducible promoter were previously generated¹⁸. The coding region for Hsc70-4 (CG4264) was cloned into pAc5.1/V5-His A (Invitrogen), and into pcDNA™6/myc-His A (Invitrogen), from amplicon produced by RT-PCR from S2Xpress RNA. Assembly was done with NEBuilder HiFi DNA Assembly Master Mix (E2621L, NEW ENGLAND BioLabs). Correct insertion of amplicon was confirmed by sequencing. The following antibodies were used: anti-Cy3/Cy5 (ab52060, Abcam), anti-dsRNA J2 (10010500, SCICONS), anti-V5 Tag (46-0705, Invitrogen), anti-6× His Tag (ab18184, Abcam), anti-α-tubulin (T5168, Sigma-Aldrich), anti-Mouse IgG (HRP) (ab6728, Abcam), anti-Mouse IgG (Alexa Fluor 488) (A11029, Invitrogen), anti-Mouse IgG (Alexa Fluor 555) (A21422, Invitrogen).

### Adaptation of S2 Cells to Insect-Xpress Medium

To generate the S2Xpress cells, S2naive cells were gradually adapted to the Insect-XPRESS protein-free medium. Briefly, S2naive cells were grown until confluency in a T25 flask in normal growth medium (Schneider's Insect Medium, supplemented with 10% FBS), and then transferred into a T75 flask, adding 5 ml of Insect-Xpress medium. 4 days after, 5 ml of Insect-XPRESS medium were added. At day 8, cells with medium were transferred into a T150 flask, and 15 ml of Insect-XPRESS medium were added. At day 11, 15 ml of cell suspension were transferred into a new T150 flask, and 15 ml of Insect-XPRESS medium were added. From this point forward, cells were adapted to the Insect-XPRESS medium, and were passaged only using this medium.

dsRNA Production

dsRNA was produced by in vitro transcription using the MEGAscript T7 Transcription kit (AM1334, Invitrogen) following the manufacturer's guidelines. For dsRNA targeting endogenous genes, cDNA produced with SuperScript™ II Reverse Transcriptase (100004925, Invitrogen) from S2Xpress RNA was used as template to amplify by PCR the target regions using primers flanked by the T7 promoter. For dsFLuc (Firefly Luciferase, GL3) and dsGFP, plasmids were used as templates (pMT-GL3 and pAc5.1-GFP). dsRNA concentration was quantified on a Qubit 3 fluorometer. All dsRNA produced against candidates were 450-600 bp in length. dsGL3: 557 bp; dsGFP: 714 bp, dsCG6647: 508 bp. The full list of primers can be found in Table 3.

TABLE 3
List of primers

| | | | |
|---|---|---|---|
| Target | Sense | Sequence (5′-3′) | SEQ ID NO: |

Production of dsRNA

| GL3 | Forward | TAATACGACTCACTATAGGGCGCCCTGGTTCCTGGAAC | 24 |
|---|---|---|---|
| GL3 | Reverse | TAATACGACTCACTATAGGGAGAATCTCACGCAGGCAGTTC | 25 |
| GFP | Forward | TAATACGACTCACTATAGGGTGGTGAGCAAGGGCGAGGAGC | 26 |
| GFP | Reverse | TAATACGACTCACTATAGGGTTGTACAGCTCGTCCATGCCG | 27 |
| CG6831 | Forward | TAATACGACTCACTATAGGGAGAACCACAGACGAAAGTGACA | 28 |
| | | CC | |
| CG6831 | Reverse | TAATACGACTCACTATAGGGAGACAGAGATTAGGCCATTGCT | 29 |
| | | GCC | |
| CG32138 | Forward | TAATACGACTCACTATAGGGAGAAGTTGCCCACCTTGAACTG | 30 |
| | | G | |
| CG32138 | Reverse | TAATACGACTCACTATAGGGAGAACGAGATACGCTCCACACG | 31 |
| | | C | |
| CG1651 | Forward | TAATACGACTCACTATAGGGAGAAAGCCGCAAAGGGTAGTCA | 32 |
| | | AC | |
| CG1651 | Reverse | TAATACGACTCACTATAGGGAGACAACCAGGTCTACTGACTGA | 33 |
| | | GC | |
| CG43140 | Forward | TAATACGACTCACTATAGGGAGAATGCCTGGCGATAAGATCCT | 34 |
| | | CA | |
| CG43140 | Reverse | TAATACGACTCACTATAGGGAGAAGAAGACCACATCGTCCCA | 35 |
| | | GTTC | |
| CG42389 | Forward | TAATACGACTCACTATAGGGAGAGCCGGAGCTTTTAGTGCCT | 36 |
| | | ATG | |
| CG42389 | Reverse | TAATACGACTCACTATAGGGAGAGCTGCCACTCCAGTGTTAGT | 37 |
| | | TG | |
| CG4264 | Forward | TAATACGACTCACTATAGGGAGAGTCTGACATGAAGCACTGG | 38 |
| | | CC | |
| CG4264 | Reverse | TAATACGACTCACTATAGGGAGAGAACTCCTGCACGAAGTGG | 39 |
| | | G | |
| CG6370 | Forward | TAATACGACTCACTATAGGGAGACAATTGGACGCTCAGTCGC | 40 |
| CG6370 | Reverse | TAATACGACTCACTATAGGGAGAGAGTCGAACGAATGCCTGA | 41 |
| | | TGG | |
| CG4169 | Forward | TAATACGACTCACTATAGGGAGAGCAACTCTTCCCGTTTCGCG | 42 |
| CG4169 | Reverse | TAATACGACTCACTATAGGGAGAACGTAGTGCAGCAGGCTCT | 43 |
| | | C | |
| CG5330 | Forward | TAATACGACTCACTATAGGGAGACGCAACACGGCCATAATGT | 44 |
| | | C | |
| CG5330 | Reverse | TAATACGACTCACTATAGGGAGAGCGCGCAAGAAGTGACCAA | 45 |
| CG10522 | Forward | TAATACGACTCACTATAGGGAGAAACCAGGCTCATCACCGTTT | 46 |
| | | C | |
| CG10522 | Reverse | TAATACGACTCACTATAGGGAGACAAATGGACATGCAGCTCA | 47 |
| | | CC | |
| CG3373 | Forward | TAATACGACTCACTATAGGGAGACGTCGGACTTCACCATCGA | 48 |
| | | AG | |
| CG3373 | Reverse | TAATACGACTCACTATAGGGAGACCACGAAGTGAACGAATCG | 49 |
| | | CTG | |
| CG4821 | Forward | TAATACGACTCACTATAGGGAGACGAGCACAACTGCAATCAC | 50 |
| | | AC | |
| CG4821 | Reverse | TAATACGACTCACTATAGGGAGACAATGAACGAATCGACCTC | 51 |
| | | GG | |
| CG8222 | Forward | TAATACGACTCACTATAGGGAGAGGAGAACCGATTGGACAGA | 52 |
| | | ATCG | |
| CG8222 | Reverse | TAATACGACTCACTATAGGGAGAACATTCAAGTGCTGTCCCAG | 53 |
| | | ATG | |
| CG9635 | Forward | TAATACGACTCACTATAGGGAGACACAACAACAGCGGTGCAG | 54 |
| | | AAC | |
| CG9635 | Reverse | TAATACGACTCACTATAGGGAGAGGCGTAAACTGATGCGACT | 55 |
| | | CGATA | |
| CG5119 | Forward | TAATACGACTCACTATAGGGAGAGCAGTACATGCGTCACATG | 56 |
| | | ACC | |
| CG5119 | Reverse | TAATACGACTCACTATAGGGAGAAACTGTGGCACTGGAGGAT | 57 |
| | | TG | |
| CG4916 | Forward | TAATACGACTCACTATAGGGAGACAAGGCATGCTGGATCATG | 58 |
| CG4916 | Reverse | TAATACGACTCACTATAGGGAGAAGTATGTCTCTGCCATTCGT | 59 |
| | | GG | |
| CG10691 | Forward | TAATACGACTCACTATAGGGAGAGGCAGCAAGGATCTGCAGA | 60 |
| | | ATG | |
| CG10691 | Reverse | TAATACGACTCACTATAGGGAGACTCAATACGTCGCAGCTCCA | 61 |
| | | C | |
| CG10686 | Forward | TAATACGACTCACTATAGGGAGACCGCAGTCGGCCAATAATC | 62 |
| CG10686 | Reverse | TAATACGACTCACTATAGGGAGAAGGACACTCCGAAGGTCTC | 63 |
| | | C | |
| CG8542 | Forward | TAATACGACTCACTATAGGGAGACGCAACACCACTATTCCCAC | 64 |
| | | CA | |
| CG8542 | Reverse | TAATACGACTCACTATAGGGAGAGCGCAGATCGGCAATTTCC | 65 |
| | | TTC | |
| CG11856 | Forward | TAATACGACTCACTATAGGGAGACCAACCTGTCGGCCAATAG | 66 |
| | | C | |
| CG11856 | Reverse | TAATACGACTCACTATAGGGAGACAACTTTACCCTCTGCGTCC | 67 |
| | | AC | |
| CG6647 | Forward | TAATACGACTCACTATAGGGAGAACCGAGGTTGCTGTCCAGG | 68 |
| | | A | |
| CG6647 | Reverse | TAATACGACTCACTATAGGGAGAGCAACTTCTGCTGGTAGCC | 69 |
| | | CAG | |
| CG6202 | Forward | TAATACGACTCACTATAGGGAGAGCGCATGTACATCCAATGG | 70 |
| | | AAC | |
| CG6202 | Reverse | TAATACGACTCACTATAGGGAGACGTAAGCAGAGCCACCAGA | 71 |
| | | A | |
| CG13387 | Forward | TAATACGACTCACTATAGGGAGACAGCGCTGCAAAGTTCCAT | 72 |
| | | C | |
| CG13387 | Reverse | TAATACGACTCACTATAGGGAGAACGCCAGAATGATGGCATG | 73 |
| | | A | |
| CG6509 | Forward | TAATACGACTCACTATAGGGAGAAGACACACGCAGCTCTTCC | 74 |
| CG6509 | Reverse | TAATACGACTCACTATAGGGAGAACGAGGCTGACATGGATCC | 75 |
| SR_CI-1 | Forward | TAATACGACTCACTATAGGGAGAGCTATCGCCGATCTATCCCA | 76 |
| | | GA | |
| SR_CI-1 | Reverse | TAATACGACTCACTATAGGGAGACGAAAGGCACTCGTCATTAC | 77 |
| | | ACC | |
| SR_CI-2 | Forward | TAATACGACTCACTATAGGGAGAAGTGCACAAAGGAGCTGCT | 78 |
| | | CA | |
| SR_CI-2 | Reverse | TAATACGACTCACTATAGGGAGAGGTACTTCCGCTCGTGTCC | 79 |
| | | A | |
| Eater-1 | Forward | TAATACGACTCACTATAGGGAGACGGGCTCCCGACAAATGTG | 80 |
| | | A | |
| Eater-1 | Reverse | TAATACGACTCACTATAGGGAGACAACACAGAATCCGTTCTTG | 81 |
| | | CACC | |
| Eater-2 | Forward | TAATACGACTCACTATAGGGAGAAAGGACGGGTGTGTCAACG | 82 |
| | | G | |
| Eater-2 | Reverse | TAATACGACTCACTATAGGGAGACATTCTTGTCCACGTGATAT | 83 |
| | | GAGCG | |
| Cloning of CG4264 (Hsc70-4) into pAc5.1/V5-His A | | | |
| CG4264 | Forward | AGACCCCGGATCGGGGTACGAAATGTCTAAAGCTCCTGCTG | 84 |
| amplicon | | | |
| CG4264 | Reverse | GGCCCTCTAGACTCGAGCGGGTCGACCTCCTCGATGG | 85 |
| amplicon | | | |
| pAc5.1- | Forward | CCGCTCGAGTCTAGAGGGC | 86 |
| V5/6His | | | |
| linearization | | | |
| pAc5.1- | Reverse | GTACCCCGATCCGGGGTC | 87 |
| V5/6His | | | |
| linearization | | | |
| Cloning of CG4264 (Hsc70-4) into pcDNA ™6/myc-His A | | | |
| CG4264 | Forward | AGTCCAGTGTGGTGGAATTCTGGAAATGTCTAAAGCTCCTGCT | 88 |
| amplicon | | G | |
| CG4264 | Reverse | CCGCCACTGTGCTGGATAGTCGACCTCCTCGATGG | 89 |
| amplicon | | | |
| pcDNA6- | Forward | TATCCAGCACAGTGGCGG | 90 |
| myc/His | | | |
| linearization | | | |
| pcDNA6- | Reverse | CAGAATTCCACCACACTGGACT | 91 |
| myc/His | | | |
| linearization | | | |
| qPCR | | | |
| CG4264 | Forward | CAGCATGGCAAGGTCGAGATC | 92 |
| CG4264 | Reverse | ATCAGACGCTCCGTATCGGTG | 93 |
| Rp49 | Forward | CGGATCGATATGCTAAGCTGT | 94 |
| Rp49 | Reverse | GCGCTTGTTCGATCCGTA | 95 |
| PCR | | | |
| CG4264 | Forward | CGCCAAGCGCTTGATTGGTC | 96 |
| CG4264 | Reverse | AGCACGCTTGTTGGTGGTCA | 97 |
| Rp49 | Forward | AAGGGTATCGACAACAGAGTGC | 98 |
| Rp49 | Reverse | ACAAATGTGTATTCCGACCACG | 99 |

Nucleic Acid Labeling with Cy3

Silencer siRNA Labeling kit—Cy3 (AM1632, Invitrogen) was used to label with Cy3 dsRNA (dsFLuc, dsGFP, dsCG6647), DNA (FLuc) and siRNA (GAPDH, from the kit), following the kit's protocol. Labeling was confirmed by a running shift of the purified product compared to unlabeled probe on a 1.5% agarose gel, and by immunofluorescence with anti-dsRNA antibody J2 (FIG. 6b-c).

### Fluorescence Microscopy

S2naive and S2Xpress cells were seeded on 8-chambers Nunc LabTek II previously coated with Poly-L-Lysine (P4832, Sigma-Aldrich), and incubated for 24 h. For silencing experiments, cells were transfected with dsRNA using Effectene Transfection reagent (301427, QIAGEN) and incubated for 72 h. For endocytosis inhibition experiments, Dynasore (D7693, Sigma-Aldrich) was added to a final concentration of 100 μM for 10-60 min at 25° C. dsRNA, DNA or siRNA labeled with Cy3 (to a final concentration of 0.76 nM) was added, and cells were incubated at 25° C. for 30-40 minutes. Cells were washed with PBS, fixed in 4% paraformaldehyde for 20 min, and blocked/permeabilized in 2% BSA-0.2% Triton X-100. Actin was visualized with Oregon Green™ 488 Phalloidin (07466, Invitrogen). For immunofluorescence with J2 and anti-Cy3 antibodies, blocking/permeabilization was done with 10% normal goat serum-0.2% Triton, following an ON incubation at 4° C. with primary antibody (J2: 1:500; anti-Cy3: 1:500). Slides were incubated with secondary antibody (anti-Mouse IgG-Alexa Fluor 488:1:1000) for 1 h, and no actin staining was performed. For immunofluorescence against rHsc70-4, cells were transfected with pAc5.1-rHsc70-4-V5/His (S2 cells) or with pcDNA6-rHsc70-4-myc/His (Hela cells), and incubated for 48 h before fixing. Staining was done was previously described, except that Triton was omitted on non-permeabilized wells. Primary antibodies were used at 1:1000 dilution for anti-V5 Tag, and 1:400 for anti-6× His. Actin was visualized with Alexa Fluor™ 555 Phalloidin (A34055, Invitrogen) (except when dsRNA-Cy3 was used). Nuclei were counterstained with DAPI. Vectashield H-1000 (Vector Laboratories, Burlingame, CA) was used as mounting medium. Imaging was done on a Leica TCS SP5 confocal microscope at 630× magnification (S2 cells) or 400× magnification (Hela cells), and processed on Fiji56.

### Luciferase Assays

Cells were seeded on 96-well culture plates and transfected with plasmids pMT-GL3 (Firefly Luciferase, 12 ng/well) and pMT-Renilla (Renilla Luciferase, 3 ng/well), and with dsRNA (10 ng/well) when indicated, using Effectene Transfection reagent (301427, QIAGEN). After 24 h, medium was changed, and dsFLuc or dsCtl (dsGFP) (50 ng/well, or the indicated amounts) was added (soaking) to allow internalization and posterior silencing of FLuc. After 24 h, plasmid expression was induced by adding CuSO₄ 10 mM. The next day, cells were lysed, and Firefly and Renilla Luciferase measurement was performed using the Dual-Luciferase Reporter Assay System (E1960, Promega) in a GLOMAX microplate luminometer. Firefly Luciferase values were normalized to Renilla Luciferase values. For analysis of medium effect on internalization, before soaking, medium was changed to the indicated medium, and soaking was allowed for 4 h. After, medium was changed back to corresponding growing medium for each cell type. Experiments for FIG. 6 were performed with minor modifications: after transfection, cells were incubated 48 h before soaking with dsRNA, and after plasmid induction with CuSO₄, cells were incubated another 48 h before cells lysis.

### Cell Surface Proteins Purification and Identification

S2naive and S2Xpress cells were seeded on T75 flaks, and incubated until the next day, when they were collected and counted. 2.4*10⁷ cells were diluted in 15 ml of growing medium. Biotinylation and purification of biotinylated proteins were done following the Pierce™ Cell Surface Biotinylation and Isolation Kit's (A44390, Thermo Scientific) protocol. Samples without biotin were used as negative controls to identify unspecific protein precipitation. Samples were eluted in 200 μl of elution buffer. 50 μl was used for proteomic analysis, and other 50 μl for gel analysis. Laemmli SDS sample buffer 6× (J60660, Alfa Aesar) (with β-mercaptoethanol) was added, and samples were boiled for 5 min at 100° C. For proteomic analysis, samples were loaded on an 7.5% SDS-PAGE (BioRad) before in-gel proteolysis. Briefly, proteins gel bands were excised and washed, and then proteins were reduced and alkylated (DTT 10 mM final, 2 h, 37° C. iodoacetamide, 50 mM final, 30 min in the dark at room temperature). After dehydration, proteins proteolysis was done with 50 ng of LysC-trypsin (Promega) at 37° C., overnight. The resulting proteolytic peptides were extracted from the gel by incubating twice for 15 min in 100 μL 1% aq. TFA and sonication, followed by one incubation of 15 min at 37° C. in 50 μL acetonitrile. Peptides were desalted using C18-filled tips (Ziptip™ C18, Millipore) and eluted in 8 μL. The digest (6 μL of peptides) was injected on a capillary reversed-phase column (C18 Acclaim PepMap100 A, 75 μm i.d., 50-cm length; Thermo Fisher Scientific) at a flow rate of 220 nL/min, with a gradient of 2% to 40% Buffer B in Buffer A in 60 min (Buffer A: H₂O/acetonitrile (ACN)/FA 98:2:0.1 (v/v/v); Buffer B: H₂O/ACN/FA10: 90:0.1 (v/v/v)). The MS analysis was performed on a Qexactive HF mass spectrometer (Thermo Fisher Scientific) with a top 10 data dependent acquisition method: MS resolution 70,000, mass range 400-2,000 Da, followed by MS/MS on the ten most intense peaks at resolution 17,500, with a dynamic exclusion for 10 s. Raw data was processed using Proteome Discoverer 2.4 (Thermo Fisher Scientific). The database search was done with Mascot search engine (Matrix Science Mascot 2.2.04) on a Drosophila protein databank (20986 entries). The SwissProt databank 2020_05 (563,552 entries) was used to assess contaminations with human proteins. The following parameters were used: MS tolerance 10 ppm; MS/MS tolerance 0.02 Da; tryptic peptides; up to two miscleavages allowed; partial modifications: carbamidomethylation C, oxidation (M), deamidation (NQ). Proteins identified by at least two high confidence peptides (FDR<0.1%) were validated. Further analysis of the identified proteins was done on FunRich software⁵⁷. For SDS-PAGE, samples were loaded on 4-15% polyacrylamide gel. After electrophoresis, gel was fixed with 40% ethanol-10% acetic acid-50% H₂O, and silver staining was performed as previously described⁵⁸.

dsRNA Binding Proteins Immunoprecipitation

S2Xpress cells were seeded on 100 mm culture plates (1*10⁷ cells/10 ml/plate) and incubated for 24 h at 25° C. Then, medium was changed, Dynasore inhibitor (D7693, Sigma-Aldrich) was added (final concentration 100 μM), and cells were incubated for 1 h at 25° C. Then, dsRNA (dsFLuc) labeled with Cy3 was added (10 μg/plate), and soaking was allowed for 45 min at 25° C. For negative control plates, unlabeled dsRNA was used. After, plates were washed in cold PBS, and UV-crosslinked (254 nm) in 3 ml of PBS (without Mg/Ca) at 300 mJ/cm² on ice in a Stratalinker UV 1800 crosslinker. Scraped cells were pelleted by centrifugation and lysed in SDS lysis buffer (0.5% SDS, 50 mM Tris, 1 mM EDTA, 1 mM DTT, pH 8, 1× Protease inhibitor (11 873 580 001, Roche)) at 65° C. for 5 min. Then, RIPA correction buffer was added (62.5 mM Tris, 1.25% NP-40 substitute, 0.625% sodium deoxycholate, 2.25 mM EDTA, 187.5 mM NaCl, pH 8, 1× Protease inhibitor), and samples were passed through QIAshredder columns (79654, QIAGEN) twice, following the manufacturer's recommendations. Then, 4 μg of antibody (anti-Cy3) was added to the samples, following an ON incubation at 4° C. on a rotator. The next day, Dynabeads™ Protein A Immunoprecipitation Kit (10006D, Invitrogen) was used to pull-down dsRNA-protein complexes binding to the antibody, following the kit's protocol. Samples were diluted and washed 5 times on RIPA buffer (50 mM Tris, 1% NP-40 substitute, 0.5% sodium deoxycholate, 0.1% SDS, 2 mM EDTA, 150 mM NaCl, pH 8, 1× Protease inhibitor (911 873 580 001, Roche)) prior to elution. To elute proteins, first samples were treated with RNase III for 1 h at 37° C., and supernatants were collected. Then, the beads were treated with Laemmli SDS sample buffer 6× (J60660, Alfa Aesar) (with 10% β-mercaptoethanol) and boiled for 5 min at 100° C., then supernatants were collected. Proteomics analysis was done as detailed in the previous section. Samples collected after RNase III treatment had only a few proteins, indicating that the elution was not effective, thus only samples collected after boiling in sample buffer were considered. Duplicates were performed for each treatment. Negative control samples were used to discard proteins that were unspecifically precipitated. Analysis of the identified proteins was done on FunRich software⁵⁷.

### High-Content Imaging Screen

Cells were seeded on black CellCarrier Ultra microplates (6055302, PerkinElmer) and transfected with 10 ng/well of dsRNA targeting candidates using Effectene Transfection reagent (301427, QIAGEN). dsCtl and Dynasore (D7693, Sigma-Aldrich) wells were transfected with dsCtl (dsFLuc). After 72 h of incubation, medium was changed, Dynasore inhibitor was added to corresponding wells (final concentration 100 μM) for 10 min at 25° C. Then, 30 ng/well of dsRNA (dsFLuc) labeled with Cy3 was added, and cells were incubated for 30 min at 25° C., then fixed in 4% paraformaldehyde for 20 min, and blocked/permeabilized in 2% BSA-0.2% Triton X-100 for 30 min. Cytoplasm was visualized with DiO dye (from Vybrant™ Multicolor Cell-Labeling Kit; V22889, Molecular Probes), and nuclei were counterstained with DAPI. Imaging was performed on an Opera Phenix microscope (PerkinElmer) at 630× magnification. Columbus software was used to design the script and analyze the images. Script was designed to quantify median intensity of Cy3 and number of spots of Cy3 in cytoplasm per well. Further analysis was done on GraphPad Prism 9. For the median fluorescence intensity, negative control wells were used to subtract baseline intensity values.

### RT-qPCR

Cells were seeded in 24-well plates and incubates for 24 h. RNA extraction was performed with TRIzol™ Reagent (15596026, Ambion), and quantified on NanoDrop One (Thermo Scientific). Equal amounts of RNA were treated with RQ1 DNase (M610A, Promega) and were used to produce cDNA with Maxima H Minus First Strand cDNA Synthesis Kit (K1682, Thermo Scientific) using random primers. quantitative PCR was done with Luminaris Color HiGreen qPCR Master Mix, low ROX (K0374, Thermo Scientific), and ran on a QuantStudio™ 7 Flex Real-Time PCR System (Applied Biosystems). Rp49 was used as a housekeeping gene. ACt values were obtained by subtracting the Ct value for Rp49 to the Ct value for the corresponding gene, and AACt values were obtained by further subtracting the geometric mean ACt value for the control condition (S2naive). Results are shown as fold change relative to S2naive. Primers sequences can be found in Table 3 above.

### PCR to Confirm Silencing

To confirm silencing of Hsc70-4 (CG4264) by dsHsc70-4, cells were transfected with dsHsc70-4 with Effectene Transfection reagent (301427, QIAGEN), and incubated for 72 h. For control condition, cells were transfected with dsCtl (dsFLuc). RNA was extracted and cDNA was produced as previously described, with the modification that Oligo(dT)₁₈ primers were used. PCR was performed with DreamTaq DNA Polymerase (EP0702, Thermo Scientific) using primers flanking the dsRNA targeting region. PCR products were loaded on a 1% agarose gel with ethidium bromide. After electrophoresis, gel was developed on a Gel Doc XR+ Imaging System (Bio-Rad). Rp49 was used as a loading control. Primers sequences can be found in Table 3 above.

### Western Blot

Cells were seeded on 6-well plates and transfected with 500 ng or 1000 ng of pAc5.1-rHsc70-4-V5/His (S2 cells) or with 100 ng of pcDNA6-rHsc70-4-myc/His (Hela cells). After 48 h, cells were washed with cold PBS, and proteins were extracted with RIPA buffer ((50 mM Tris, 1% NP-40 substitute, 0.5% sodium deoxycholate, 0.1% SDS, 2 mM EDTA, 150 mM NaCl, pH 8, 1× Protease inhibitor (11 873 580 001, Roche)). Samples were incubated on ice for 20 min, and centrifuged for 20 min at 4° C. 13000 g. Supernatants were collected. To confirm expression of recombinant protein, equal volumes of samples were boiled in XT Sample Buffer (161-0791, Bio-Rad) with β-mercaptoethanol for 5 min, and loaded on a 4-20% Mini-PROTEAN® TGX Stain-Free™ Protein Gel (4568094, Bio-Rad). PageRuler™ Prestained Protein Ladder (26616, Thermo Scientific) was used as a molecular weight ladder. After electrophoresis, gel was activated and then transferred for 30 min to a Trans-Blot Turbo Mini 0.2 μm nitrocellulose membrane (1704158, Bio-Rad) on a Trans-Blot Turbo transfer system (BioRad). Total proteins were visualized by UV on a Gel Doc XR+ Imaging System (Bio-Rad). Membranes were blocked with 5% non-fat dry milk in PBS-0.05% Tween and incubated ON with primary antibody at 4° C. (anti-V5 Tag: 1:5000, anti-6× His: 1:5000, anti-α-tubulin: 1:5000). Washes were done with PBS-0.05% Tween. Membranes were incubated for 1:30 h with secondary antibody (anti-Mouse IgG (HRP): 1:5000), developed with SuperSignal™ West Pico PLUS Chemiluminescent Substrate (34580, Thermo Scientific), and imaged on a G: BOX Chemi XL (Syngene). For silencing experiments, cells were co-transfected with 500 ng of plasmid and 100 ng of dsHsc70-4 or dsCtl (dsFLuc), and incubated for 1, 2, or 3 days. Sample's protein concentration was quantified with Pierce™ BCA Protein Assay Kit (23227, Thermo Scientific), and equal amount of proteins were loaded on the gel. α-Tubulin was used as a loading control.

### Electrophoretic Mobility Shift Assay (EMSA)

For EMSA experiments, dsRNA (dsFLuc, dsCG6647) DNA (FLuc), and siRNA (GAPDH) labeled with Cy3 were used as probes. Hsc70-4 recombinant protein was produced by GenScript by expression of recombinant protein with 6× His tag on E. coli. Protein was purified with Ni resin. Labeled-probe (0.76 nM) was incubated with different concentrations of recombinant protein in binding buffer (25 mM Tris, 50 mM NaCl. 0.1 mg/ml BSA, 31.25 mM DTT, 0.625 mg/ml tRNA E. coli; 20 μl reaction) for 30 min at 25° C. Native loading buffer was added after incubation (100 mM Tris, 10% glycerol, 0.0025% bromophenol blue, pH: 8). For competition assays, 10 times more of the same unlabeled-dsRNA was added to the reaction mix. EMSA gels (native 4% polyacrylamide, 0.5×TBE) were pre-run at 200 V on ice prior to loading samples on 0.5×TBE buffer. Samples were loaded, and ran at 200 V on ice. Gels were imaged by fluorescence detection on a Typhoon™ FLA 9000 (GE Healthcare).

### Statistical Analysis

Data are represented as mean±SD. Statistical analysis were done on GraphPad Prism version 9 (GraphPad Software, CA, USA). Two-tailed unpaired t-test (2 groups) or one-way ANOVA (3 or more groups) followed by Dunnett's post-hoc test was used to detect significant differences between groups. p-values<0.05 were considered significant. Normality and homoscedasticity were assumed for data. Whenever these criteria were not met, non-parametrical Welch's ANOVA test was used (3 or more groups) followed by Dunnett's T3 post-hoc test. For cellular component analysis of proteomic results, Hypergeometric test was done by FunRich software⁵⁷.

### DESCRIPTION OF THE SEQUENCES

### Hsc70-4 Protein Sequences

heat shock protein cognate 4, isoform G [Drosophila melanogaster] 651 aa protein—Accession: NP_001262586.1/AGB95967.1/GI: 442619165 [SEQ ID NO. 1]
heat shock protein cognate 4, isoform F [Drosophila melanogaster] 651 aa Protein—Accession: NP_788680.1/AAO41568.1/GI: 28571721 [SEQ ID NO. 2]
heat shock protein cognate 4, isoform E [Drosophila melanogaster] 651 aa Protein—Accession: NP_788679.1/AAO41567.1/GI: 28571719 [SEQ ID NO. 3]
heat shock protein cognate 4, isoform D [Drosophila melanogaster] 651 aa protein Accession: NP_731989.1/AAN13639.1/GI: 24647038 [SEQ ID NO. 4]
heat shock protein cognate 4, isoform C [Drosophila melanogaster] 651 aa protein—Accession: NP_731988.1/AAN13638.1/GI: 24647036 [SEQ ID NO. 5]
heat shock protein cognate 4, isoform B [Drosophila melanogaster] 651 aa protein—Accession: NP_731987.1/AAN13637.1/GI: 24647034 [SEQ ID NO. 6]
heat shock protein cognate 4, isoform A [Drosophila melanogaster] 651 aa protein—Accession: NP_524356.1/AAF55150.1/GI: 17737967 [SEQ ID NO. 7]
rHsc70-4 amino acid sequence from pAc5.1-rHsc70-4-V5/His (683 aa) [SEQ ID NO. 8] (for expression in S2 cells)

MSKAPAVGIDLGTTYSCVGVFQHGKVEIIANDQGNRTTPSYVAFTDTER
LIGDAAKNQVAMNPTQTIFDAKRLIGRKFDDAAVQSDMKHWPFEVVSAD
GKPKIEVTYKDEKKTFFPEEISSMVLTKMKETAEAYLGKTVTNAVITVP
AYFNDSQRQATKDAGTIAGLNVLRIINEPTAAAIAYGLDKKAVGERNVL
IFDLGGGTFDVSILSIDDGIFEVKSTAGDTHLGGEDFDNRLVTHFVQEF
KRKHKKDLTTNKRALRRLRTACERAKRTLSSSTQASIEIDSLFEGTDFY
TSITRARFEELNADLFRSTMDPVEKALRDAKLDKSVIHDIVLVGGSTRI
PKVQRLLQDLFNGKELNKSINPDEAVAYGAAVQAAILHGDKSQEVQDLL
LLDVTPLSLGIETAGGVMSVLIKRNTTIPTKQTQTFTTYSDNQPGVLIQ
VYEGERAMTKDNNLLGKFELSGIPPAPRGVPQIEVTFDIDANGILNVTA
LERSTNKENKITITNDKGRLSKEDIERMVNEAEKYRNEDEKQKETIAAK
NGLESYCFNMKATLDEDNLKTKISDSDRTTILDKCNETIKWLDANQLAD
KEEYEHRQKELEGVCNPIITKLYQGAGFPPGGMPGGPGGMPGAAGAAGA
AGAGGAGPTIEEVDPLESRGPFEGKPIPNPLLGLDSTRTGHHHHHH*

rHsc70-4 amino acid sequence from pcDNA6-rHsc70-4-myc/His (686 aa) [SEQ ID NO. 9] (for expression in Hela cells)

MSKAPAVGIDLGTTYSCVGVFQHGKVEIIANDQGNRTTPSYVAFTDTE
RLIGDAAKNQVAMNPTQTIFDAKRLIGRKFDDAAVQSDMKHWPFEVVS
ADGKPKIEVTYKDEKKTFFPEEISSMVLTKMKETAEAYLGKTVTNAVI
TVPAYFNDSQRQATKDAGTIAGLNVLRIINEPTAAAIAYGLDKKAVGE
RNVLIFDLGGGTFDVSILSIDDGIFEVKSTAGDTHLGGEDFDNRLVTH
FVQEFKRKHKKDLTTNKRALRRLRTACERAKRTLSSSTQASIEIDSLF
EGTDFYTSITRARFEELNADLFRSTMDPVEKALRDAKLDKSVIHDIVL
VGGSTRIPKVQRLLQDLFNGKELNKSINPDEAVAYGAAVQAAILHGDK
SQEVQDLLLLDVTPLSLGIETAGGVMSVLIKRNTTIPTKQTQTFTTYS
DNQPGVLIQVYEGERAMTKDNNLLGKFELSGIPPAPRGVPQIEVTFDI
DANGILNVTALERSTNKENKITITNDKGRLSKEDIERMVNEAEKYRNE
DEKQKETIAAKNGLESYCFNMKATLDEDNLKTKISDSDRTTILDKCNE
TIKWLDANQLADKEEYEHRQKELEGVCNPIITKLYQGAGFPPGGMPGG
PGGMPGAAGAAGAAGAGGAGPTIEEVDYPAQWRPLESRGPFEQKLISE
EDLNMHTGHHHHHH*

rHsc70-4 produced commercially by GenScript, from E. coli (657 aa) [SEQ ID NO. 10]

MSKAPAVGIDLGTTYSCVGVFQHGKVEIIANDQGNRTTPSYVAFTDTER
LIGDAAKNQVAMNPTQTIFDAKRLIGRKFDDAAVQSDMKHWPFEVVSAD
GKPKIEVTYKDEKKTFFPEEISSMVLTKMKETAEAYLGKTVTNAVITVP
AYFNDSQRQATKDAGTIAGLNVLRIINEPTAAAIAYGLDKKAVGERNVL
IFDLGGGTFDVSILSIDDGIFEVKSTAGDTHLGGEDFDNRLVTHFVQEF
KRKHKKDLTTNKRALRRLRTACERAKRTLSSSTQASIEIDSLFEGTDFY
TSITRARFEELNADLFRSTMDPVEKALRDAKLDKSVIHDIVLVGGSTRI
PKVQRLLQDLFNGKELNKSINPDEAVAYGAAVQAAILHGDKSQEVQDLL
LLDVTPLSLGIETAGGVMSVLIKRNTTIPTKQTQTFTTYSDNQPGVLIQ
VYEGERAMTKDNNLLGKFELSGIPPAPRGVPQIEVTFDIDANGILNVTA
LERSTNKENKITITNDKGRLSKEDIERMVNEAEKYRNEDEKQKETIAAK
NGLESYCFNMKATLDEDNLKTKISDSDRTTILDKCNETIKWLDANQLAD
KEEYEHRQKELEGVCNPIITKLYQGAGFPPGGMPGGPGGMPGAAGAAGA
AGAGGAGPTIEEVDHHHHHH*

mRNA sequences:
Transcript variant A (Hsc70-4), mRNA, NCBI Reference Sequence: NM_079632.6 (2368 bp)=[SEQ ID NO:100]
Transcript variant B (Hsc70-4), mRNA, NCBI Reference Sequence: NM_169625.2 (2339 bp)=[SEQ ID NO:101]

Transcript variant C (Hsc70-4), mRNA, NCBI Reference Sequence: NM_169626.2 (2280 bp)=[SEQ ID NO:102]
Transcript variant D (Hsc70-4), mRNA, NCBI Reference Sequence: NM_169627.2 (2352 bp)=[SEQ ID NO:103]
Transcript variant E (Hsc70-4), mRNA, NCBI Reference Sequence: NM_176502.2 (2284 bp)=[SEQ ID NO:104]
Transcript variant F (Hsc70-4), mRNA, NCBI Reference Sequence: NM_176503.2 (2359 bp)=[SEQ ID NO:105]
Transcript variant G (Hsc70-4), mRNA, NCBI Reference Sequence: NM_001275657.1 (2336 bp)=[SEQ ID NO:106]

### Gene

Gene encoding the D. melanogaster HSC70-4 protein, accession number CG4264, FlyBase accession number FBgn0266599 (3928 bp)=[SEQ ID NO:23]
Coding Sequence (CDS) for all mRNA transcripts (1956 bp)=[SEQ ID NO:107]

ATGTCTAAAGCTCCTGCTGTTGGTATTGATTTGGGCACCACCTACTCGT
GCGTGGGCGTGTTCCAGCATGGCAAGGTCGAGATCATCGCCAACGACCA
GGGTAATCGTACCACTCCATCCTATGTTGCCTTCACCGATACGGAGCGT
CTGATCGGAGATGCCGCCAAGAACCAGGTGGCGATGAACCCGACCCAGA
CGATCTTCGACGCCAAGCGCTTGATTGGTCGCAAGTTCGATGATGCCGC
CGTGCAGTCTGACATGAAGCACTGGCCCTTCGAGGTGGTCAGCGCCGAT
GGCAAGCCCAAGATCGAGGTGACCTACAAGGACGAGAAGAAGACCTTCT
TCCCCGAGGAGATCTCTTCGATGGTGCTTACCAAGATGAAGGAGACCGC
CGAGGCCTATCTGGGCAAGACTGTGACCAACGCGGTCATCACCGTGCCG
GCCTACTTCAACGACTCTCAGCGTCAGGCGACCAAGGACGCCGGCACCA
TCGCCGGTCTGAACGTGCTGCGTATCATCAACGAGCCCACTGCCGCTGC
TATCGCTTACGGTCTGGACAAGAAGGCTGTTGGAGAGCGCAACGTGCTC
ATCTTCGATCTGGGCGGCGGCACCTTCGATGTGTCCATCCTGTCGATCG
ATGACGGTATCTTTGAGGTCAAGTCCACGGCCGGAGATACGCATCTGGG
TGGTGAGGACTTCGACAACCGTCTGGTCACCCACTTCGTGCAGGAGTTC
AAGCGCAAGCACAAGAAGGATCTGACCACCAACAAGCGTGCTCTGCGTC
GTCTGCGCACCGCTTGCGAGCGTGCAAAGCGTACCCTGTCGTCCTCCAC
CCAGGCCAGCATTGAGATCGACTCTCTGTTCGAGGGTACCGACTTCTAC
ACCTCGATTACTCGTGCCCGTTTCGAGGAGTTGAACGCTGATCTGTTCC
GCAGCACCATGGACCCCGTGGAGAAGGCTCTGCGTGACGCCAAGCTGGA
CAAGTCGGTCATCCACGACATTGTGCTGGTCGGTGGCTCCACCCGTATC
CCCAAGGTGCAGCGCCTGCTGCAGGATCTGTTCAATGGCAAGGAGCTGA
ACAAGTCGATCAATCCCGATGAGGCTGTGGCCTACGGTGCTGCCGTCCA
GGCCGCCATTCTGCACGGCGACAAGTCGCAGGAGGTGCAGGATCTGCTG
CTGCTCGATGTGACTCCTCTGTCCCTGGGTATCGAAACCGCTGGCGGTG
TGATGAGCGTGTTGATCAAGCGCAACACCACCATCCCGACCAAGCAGAC
CCAGACCTTCACCACCTACTCGGACAACCAGCCCGGTGTGCTGATCCAG
GTGTACGAGGGAGAGCGTGCCATGACCAAGGACAACAACCTGCTCGGCA
AGTTCGAGCTGTCGGGCATCCCCCCCGCACCACGTGGTGTGCCCCAGAT
CGAGGTCACCTTCGATATCGATGCCAACGGTATCCTCAACGTGACTGCC
CTGGAGCGTTCGACCAACAAGGAGAACAAGATCACCATTACCAACGACA
AGGGTCGTCTCTCCAAGGAGGACATCGAGCGCATGGTCAACGAGGCCGA
GAAGTACCGCAACGAGGATGAGAAGCAGAAGGAGACCATTGCCGCCAAG
AACGGCCTCGAGTCGTACTGCTTCAACATGAAGGCCACCCTCGACGAGG
ATAACCTGAAGACCAAGATCTCGGACTCTGACCGCACCACAATCCTGGA
CAAGTGCAACGAGACCATCAAGTGGCTGGATGCCAACCAGCTGGCTGAC
AAGGAGGAGTACGAGCACCGCCAGAAGGAACTGGAGGGTGTGTGCAACC
CGATCATTACCAAGCTATACCAGGGCGCCGGTTTCCCACCCGGTGGCAT
GCCCGGCGGTCCCGGAGGTATGCCCGGAGCGGCTGGTGCCGCTGGCGCT
GCCGGAGCCGGCGGTGCTGGCCCCACCATCGAGGAGGTCGACTAA

### Plasmids:

pAc5.1-rHsc70-4-V5/His (plasmid sequence-top strand) for expression in S2 cells (7282 bp)=[SEQ ID NO:18]
pcDNA6-rHsc70-4-myc/His (plasmid sequence top-strand) for expression in vertebrate cells (Hela cells) (7078 bp)=[SEQ ID NO:19]

### Ds Sequences:

dsHsc70-4 target sequence (top-strand, 484 bp)=[SEQ ID NO:20]
dsFLuc target sequence (top-strand, 560 bp)=[SEQ ID NO:21]
dsGFP target sequence (top-strand, 715 bp)=[SEQ ID NO:22]
dsMI_CRT target sequence=[SEQ ID NO:109]
dsCath-L target sequence=[SEQ ID NO:110]

### REFERENCES

1. Riera Romo, M., Pérez-Martínez, D. & Castillo Ferrer, C. Innate immunity in vertebrates: An overview. Immunology 148, 125-139 (2016).
2. Ferrandon, D., Imler, J. L., Hetru, C. & Hoffmann, J. A. The Drosophila systemic immune response: Sensing and signalling during bacterial and fungal infections. Nature Reviews Immunology 7, 862-874 (2007).
3. Grant, M. & Lamb, C. Systemic immunity. Curr. Opin. Plant Biol. 9, 414-420 (2006).
4. Medzhitov, R. & Janeway, C. A. Innate immunity: impact on the adaptive immune response. Curr Opin Immuno/9, 4-9 (1997).
5. Majzoub, K., Wrensch, F. & Baumert, T. F. The Innate Antiviral Response in Animals: An Evolutionary Perspective from Flagellates to Humans. Viruses 11, (2019).
6. Rehwinkel, J. & Gack, M. U. RIG-I-like receptors: their regulation and roles in RNA sensing. Nat. Rev. Immunol. 20, 537-551 (2020).
7. Brisse, M. & Ly, H. Comparative structure and function analysis of the RIG-I-like receptors: RIG-I and MDA5. Front. Immunol. 10, 1586 (2019).
8. Marques, J. T. & Imler, J. L. The diversity of insect antiviral immunity: Insights from viruses. Curr. Opin. Microbiol. 32, 71-76 (2016).
9. Kingsolver, M. B., Huang, Z. & Hardy, R. W. Insect antiviral innate immunity: Pathways, effectors, and connections. J. Mol. Biol. 425, 4921-4936 (2013).
10. Karlikow, M., Goic, B. & Saleh, M. C. RNAi and antiviral defense in Drosophila: Setting up a systemic immune response. Dev. Comp. Immunol. 42, 85-92 (2014).
11. Kemp, C. et al. Broad RNA Interference-Mediated Antiviral Immunity and Virus-Specific Inducible Responses in Drosophila. J. Immunol. 190, 650-658 (2013).
12. Mukherjee, S. & Hanley, K. A. RNA interference modulates replication of dengue virus in Drosophila melanogaster cells. BMC Microbiol. 10, 127 (2010).
13. Schneider, D. USING DROSOPHILA AS A MODEL INSECT. Nat. Rev. Genet. 1, 218-226 (2000).
14. Van Rij, R. P. et al. The RNA silencing endonuclease Argonaute 2 mediates specific antiviral immunity in Drosophila melanogaster. Genes Dev. 20, 2985-2995 (2006).
15. Saleh, M. C. et al. Antiviral immunity in Drosophila requires systemic RNA interference spread. Nature 458, 346-350 (2009).
16. Miller, S. C., Brown, S. J. & Tomoyasu, Y. Larval RNAi in Drosophila? Dev. Genes Evol. 218, 505-510 (2008).
17. Tassetto, M., Kunitomi, M. & Andino, R. Circulating Immune Cells Mediate a Systemic RNAi-Based Adaptive Antiviral Response in Drosophila. Cell 169, 314-325.e13 (2017).
18. Saleh, M.-C. et al. The endocytic pathway mediates cell entry of dsRNA to induce RNAi silencing. Nat. Cell Biol. 8, 793 (2006).
19. Ulvila, J. et al. Double-stranded RNA is internalized by scavenger receptor-mediated endocytosis in Drosophila S2 cells. J. Biol. Chem. 281, 14370-14375 (2006).
20. McEwan, D. L., Weisman, A. S. & Hunter, C. P. Uptake of Extracellular Double-Stranded RNA by SID-2. Mol. Cell 47, 746-754 (2012).
21. Limmon, G. V et al. Scavenger receptor class-A is a novel cell surface receptor for double-stranded RNA. FASEB J. 22, 159-167 (2008).
22. Nellimarla, S. & Mossman, K. L. Extracellular dsRNA: Its function and mechanism of cellular uptake. J. Interf. Cytokine Res. 34, 419-426 (2014).
23. Zhou, H. et al. CD11b/CD18 (Mac-1) Is a Novel Surface Receptor for Extracellular Double-Stranded RNA To Mediate Cellular Inflammatory Responses. J. Immunol. 190, 115-125 (2013).
24. Yanagawa, S. I., Lee, J. S. & Ishimoto, A. Identification and characterization of a novel line of Drosophila Schneider s2 cells that respond to wingless signaling. J. Biol. Chem. 273, 32353-32359 (1999).
25. Luhur, A., Mariyappa, D., Klueg, K. M., Roberts, J. & Zelhof, A. C. Serum-free adapted Drosophila S2R+ line is amenable to RNA interference. microPublication Biol. 2021, (2021).
26. Vodovar, N., Goic, B., Blanc, H. & Saleh, M.-C. Silico Reconstruction of Viral Genomes from Small RNAs Improves Virus-Derived Small Interfering RNA Profiling. J. Virol. 85, (2011).
27. Goic, B. et al. RNA-mediated interference and reverse transcription control the persistence of RNA viruses in the insect model Drosophila. Nat. Immunol. 14, 396-403 (2013).
28. Chang, K. Y. & Ramos, A. The double-stranded RNA-binding motif, a versatile macromolecular docking platform. FEBS J. 272, 2109-2117 (2005).
29. Hur, S. Double-Stranded RNA Sensors and Modulators in Innate Immunity. Annual Review of Immunology 37, 349-375 (2019).
30. Montes de Oca Balderas, P. Mitochondria-plasma membrane interactions and communication. J. Biol. Chem. 297, 101164 (2021).
31. Westermann, B. The mitochondria-plasma membrane contact site. Curr. Opin. Cell Biol. 35, I-6 (2015).
32. Fridolfsson, H. N. et al. Mitochondria-localized caveolin in adaptation to cellular stress and injury. FASEB J. 26, 4637-4649 (2012).
33. Poria, D. & Ray, P. RNA-protein UV-crosslinking Assay. Bio-Protocol 7, 1-8 (2017).
34. Sei, E. & Conrad, N. K. UV cross-linking of interacting RNA and protein in cultured cells. Methods in Enzymology 539, (2014).
35. Darnell, J. C., Mele, A., Hung, K. Y. S. & Darnell, R. B. Ultraviolet (UV) cross-linking of live cells, lysate preparation, and RNase titration for cross-linking immunoprecipitation (CLIP). Cold Spring Harb. Protoc. 2018, 971-977 (2018).
36. Liu, J. et al. Argonaute2 is the catalytic engine of mammalian RNAi. Science (80-.). 305, 1437-1441 (2004).
37. Lindquist, S. & Craig, E. A. THE HEAT-SHOCK PROTEINS. Annu. Rev. Genet. 22, 631-77 (1988).
38. Suzuki, T. et al. Presence of molecular chaperones, heat shock cognate (Hsc) 70 and heat shock proteins (Hsp) 40, in the postsynaptic structures of rat brain. Brain Res. 816, 99-110 (1999).
39. Kumar, A. & Tiwari, A. K. Molecular Chaperone Hsp70 and Its Constitutively Active Form Hsc70 Play an Indispensable Role During Eye Development of Drosophila melanogaster. Mol. Neurobiol. 55, 4345-4361 (2018).
40. Bonam, S. R., Ruff, M. & Muller, S. HSPA8/HSC70 in Immune Disorders: A Molecular Rheostat that Adjusts Chaperone-Mediated Autophagy Substrates. Cells 8, (2019).
41. Pujhari, S. et al. Heat shock protein 70 (Hsp70) mediates Zika virus entry, replication, and egress from host cells. Emerg. Microbes Infect. 8, 8-16 (2019).
42. Iwasaki, S. et al. Hsc70/Hsp90 chaperone machinery mediates ATP-dependent RISC loading of small RNA duplexes. Mol. Cell 39, 292-299 (2010).
43. Bronk, P. et al. Drosophila Hsc70-4 is critical for neurotransmitter exocytosis in vivo. Neuron 30, 475-488 (2001).
44. Merkling, S. H. et al. The heat shock response restricts virus infection in Drosophila. Sci. Rep. 5, 1-15 (2015).
45. Chang, H. C. et al. Hsc70 is required for endocytosis and clathrin function in Drosophila. J. Cell Biol. 159, 477-487 (2002).
46. Uytterhoeven, V. et al. Hsc70-4 Deforms Membranes to Promote Synaptic Protein Turnover by Endosomal Microautophagy. Neuron 88, 735-748 (2015).
47. Johnson, S. L., Ranxhi, B., Libohova, K., Tsou, W. L. & Todi, S. V. Ubiquitin-interacting motifs of ataxin-3 regulate its polyglutamine toxicity through hsc70-4-dependent aggregation. Elife 9, 1-22 (2020).
48. Wang, L. et al. Heat Shock Protein Member 8 (HSPA8) Is Involved in Porcine Reproductive and Respiratory Syndrome Virus Attachment and Internalization. Microbiol. Spectr. 10, (2022).
49. Schulz, C. & Becker, S. C. Mosquitoes as Arbovirus Vectors: From Species Identification to Vector Competence. in Mosquito-borne Diseases: Implications for Public Health 163-212 (2018). doi: 10.1007/978-3-319-94075-5_9.
50. McFarlane, M. et al. The Aedes aegypti Domino Ortholog p400 Regulates Antiviral Exogenous Small Interfering RNA Pathway Activity and ago-2 Expression. mSphere 5, (2020).
51. Kang, S. et al. Optimization of double-stranded RNAi intrathoracic injection method in Aedes aegypti. Entomol. Res. 48, 269-278 (2018).
52. Maori, E. et al. A Transmissible RNA Pathway in Honey Bees. Cell Rep. 27, 1949-1959.e6 (2019).
53. Hunter, W., Ellis, J., Vanengelsdorp, D., Hayes, J. & Westervelt, D. Large-Scale Field Application of RNAi Technology Reducing Israeli Acute Paralysis Virus Disease in Honey Bees (Apis mellifera, Hymenoptera: Apidae). PLoS Pathog 6, 1001160 (2010).
54. Zotti, M. et al. RNA interference technology in crop protection against arthropod pests, pathogens and nematodes. Pest Manag. Sci. 74, 1239-1250 (2018).
55. Garbian, Y., Maori, E., Kalev, H., Shafir, S. & Sela, I. Bidirectional Transfer of RNAi between Honey Bee and Varroa destructor: Varroa Gene Silencing Reduces Varroa Population. PLoS Pathog. 8, (2012).
56. Schindelin, J. et al. Fiji: An open-source platform for biological-image analysis. Nat. Methods 9, 676-682 (2012).
57. Fonseka, P., Pathan, M., Chitti, S. V., Kang, T. & Mathivanan, S. FunRich enables enrichment analysis of OMICs datasets. J. Mol. Biol. 433, (2021).
58. Mortz, E., Krogh, T. N., Vorum, H. & Gorg, A. Improved silver staining protocols for high sensitivity protein identification using matrix-assisted laser desorption/ionization-time of flight analysis. Proteomics 1, 1359-1363 (2001)
59. Rosso et al. 2005, MPMI Vol. 18, No. 7, 2005, pp. 615-620. DOI: 10.1094/MPMI-18-0615
60. Jaouannet et al., 2013 MPMI Vol 26, No. 1, 2013, pp. 97-105. http://dx.doi.org/10.1094/MPMI-05-12-0130-R
61. Jaubert-Possamai et al. 2007. BMC Biology, 10.1186/1472-6750-7-63

1. A recombinant DNA polynucleotide comprising a coding polynucleotide operably linked to a promoter that is functional in a plant cell, wherein said polynucleotide encodes an insect heat shock protein cognate 4 (Hsc70-4), having at least 80% identity with at least one amino acid sequence chosen among SEQ ID NO:1-10.

2. (canceled)

3. A transgenic plant, seed, or plant cell comprising the recombinant DNA polynucleotide of claim 1.

4. (canceled)

5. The transgenic plant, seed, or plant cell of claim 3, further comprising a nucleic acid comprising an expressable RNA interference construct encoding a dsRNA molecule capable of down-regulating or suppressing the expression of at least one gene of a pathogen that is capable of infecting the plant.

6. The transgenic plant, plant seed, or plant cell of claim 3, wherein its genome has been modified using a genome modification technique selected from the group consisting of a polynucleotide-guided endonuclease, CRISPR-Cas endonucleases, base editing deaminases, a zinc finger nuclease, a transcription activator-like effector nuclease (TALEN), and engineered site-specific meganucleases, or Argonaute.

7. A method of increasing pest disease tolerance or resistance in a plant, seed, or plant cell comprising expressing in said plant, plant seed, or plant cell the polynucleotide of claim 1, wherein said plant, plant seed, or plant cell has increased disease resistance or tolerance when compared to a control plant, plant seed, or plant cell not comprising the polynucleotide.

8. (canceled)

9. (canceled)

10. A pest inhibitory composition comprising an insect heat shock protein cognate 4 (Hsc70-4) protein and a dsRNA molecule capable of down-regulating or suppressing the expression of at least one gene of a pathogen that is capable of infecting the plant.

11. (canceled)

12. (canceled)

13. (canceled)

14. (canceled)

15. (canceled)

16. (canceled)

17. A method of reducing resistance development to an interfering RNA molecule in a pest population, said method comprising increasing the level of an Hsc70-4 protein in the pest population by expressing an insect heat shock protein cognate 4 (Hsc70-4) protein having at least 80% identity with at least one amino acid sequence selected from SEQ ID NO:1-10 and an interfering RNA which inhibits expression of a target gene in a larval and adult pest, in a transgenic plant fed upon by the pest population.

18. (canceled)

19. (canceled)

20. (canceled)

21. (canceled)

22. (canceled)

23. (canceled)

24. (canceled)

25. A method of controlling a pest, said method comprising contacting said pest with an inhibitory amount of a combination of a dsRNA and an insect heat shock protein cognate 4 (Hsc70-4) protein having at least 80% identity with at least one amino acid sequence selected from SEQ ID NO:1-10.

26. The method of claim 25, wherein the Hsc70-4 protein comprises an one amino acid sequence selected from SEQ ID NO:1-10.

27. The method of claim 25, further comprising applying the Hsc70-4 protein to a plant susceptible to the pest.

28. The method of claim 25, further comprising expressing the Hsc70-4 protein in transgenic cells of a plant susceptible to the pest.

29. The method of claim 28, wherein the Hsc70-4 protein is secreted from the transgenic cells of the plant.

30. The method of claim 28, wherein transgenic cells of a plant comprise a recombinant DNA polynucleotide comprising a polynucleotide encoding the Hsc70-4 protein operably linked to a promoter that is functional in the plant cells.

31. The method of claim 25, wherein the pest is further contacted with a supplemental agent selected from pyrethrins and synthetic pyrethroids; oxadizine derivatives; chloronicotinyls; nitroguanidine derivatives; triazoles; organophosphates; pyrrols; pyrazoles; phenyl pyrazoles; diacylhydrazines; biological/fermentation products; and carbamates.

32. The method of claim 27, comprising spraying the Hsc70-4 protein onto leaves of the plant.

33. The method of claim 27, comprising submergence of root system in the Hsc70-4 protein.

34. The method of claim 28, comprising delivery by a nanoparticle- or Agrobacterium-mediated delivery system.
